(19) United States
(12) Patent Application Publication
(10) Pub. No.: US 2024/0074473 A1
(43) Pub. Date: Mar. 7, 2024

(54) METHOD FOR INDUCING HYPERTROPHIC MUSCLE FIBERS FOR INDUSTRIAL MEAT PRODUCTION

(71) Applicant: Yeda Research and Development Co. Ltd., Rehovot (IL)

(72) Inventors: Eldad TZAHOR, Rehovot (IL); Ori PORAT-AVINOAM, Rehovot (IL); Tamar Miriam Rose EIGLER-HIRSH, Givat Shmuel (IL)

(73) Assignee: Yeda Research and Development Co. Ltd., Rehovot (IL)

(21) Appl. No.: 18/387,165

(22) Filed: Nov. 6, 2023

Related U.S. Application Data
(63) Continuation of application No. PCT/IL2022/050474, filed on May 5, 2022.
(60) Provisional application No. 63/283,242, filed on Nov. 25, 2021.

(30) Foreign Application Priority Data
May 6, 2021 (IL) 283011

(51) Int. Cl. A23L 13/40; C12N 5/077

(52) U.S. Cl. CPC A23L 13/45; C12N 5/0658; C12N 2500/14; C12N 2501/50; C12N 2501/727; C12N 2510/00

(57) ABSTRACT

A method of inducing multinucleated myotube formation is provided. The method comprising contacting myogenic precursor cells from a farmed animal with an Extracellular Regulated Signaling Kinase (ERK1/2) inhibitor and/or an upregulator of intracellular Ca 2+ and/or RXR/RAR agonists, enhancing fusion and myogenic maturation.

### RELATED APPLICATIONS

This application is a Continuation of PCT Patent Application No. PCT/IL2022/050474 having International filing date of May 5, 2022, which claims the benefit of priority of U.S. Provisional Patent Application No. 63/283,242 filed on Nov. 25, 2021 and Israel Patent Application No. 283011 filed on May 6, 2021. The contents of the above applications are all incorporated by reference as if fully set forth herein in their entirety.

### FIELD AND BACKGROUND OF THE INVENTION

The present invention, in some embodiments thereof, relates to methods for cell culture and, more particularly, but not exclusively, to cultured meat.

The meat industry is one of the largest contributors to environmental stress, through pollution, through fossil fuel usage, methane and other waste production, as well as water and land consumption. In parallel, the global population is estimated to reach nearly 9.7 billion by the year 2050, and 11 billion by 2100 and with that increase will come an increased demand for meat products, a demand that is not sustainable by the current environmental situation. Therefore, alternative meat sources are essential.

Meat, in common usage, is comprised primarily of muscle tissue. The concept of cultured meat, or in vitro meat, or laboratory grown meat, is based on techniques that have been used in the laboratory setting for many years in the field of investigation of processes related to muscle biology. In simple terms, a muscle biopsy is harvested and enzymatically dissociated. Then the muscle precursor (stem) cells are isolated and expanded by several orders of magnitude in growth conditions (i.e. proliferation medium). Then, once enough cells have been obtained, they are transferred into reduced serum media (differentiation media), which leads to their eventual cell-cycle exit, initiation of a muscle differentiation program, and finally the fusion of myoblasts to form multinucleated myotubes. Myotubes are similar to adult muscle fibers found in the original organism. Therefore, myotubes achieved through this process are considered equivalent to meat.

The process of myoblast proliferation→differentiation→fusion is complex, yet several molecular signaling pathways have been implicated in regulating various components of this process. The cultured meat industry takes advantage of this well characterized process and utilizes this differentiation scheme in order to generate multinucleated myotubes from either primary derived myoblasts or muscle cell lines on the large scale. This is typically accomplished by expanding large numbers of precursor cells in bio-reactors over time (30-40 days) and then collecting the cells and seeding them onto a surface while simultaneously changing them from proliferation media to differentiation media and allowing differentiation and fusion to proceed spontaneously until multinucleated myotubes are acquired. Currently, the process of in vitro differentiation and myotube formation is very inefficient and time consuming. The time until myotube formation varies depending on the original species of the muscle tissue (i.e avian, between 4-6 days; bovine, between 10-14 days). The use of molecules which target mechanisms which specifically activate differentiation, and enhance myoblast fusion and multinucleated myotube formation may enhance the efficiency and thus overall productivity/yield of the cultured meat industry.

The mitogen-activated protein kinases (MAPK), including p38, JNK, ERK1/2 and ERK 5, mediate diverse signaling pathways, and are all implicated in muscle development and myoblast differentiation. The role of ERK1/2 in muscle differentiation and fusion remains unclear as both positive and negative roles have been suggested. ERK1/2 promotes myoblast proliferation in response to various growth factors; inhibition of signaling pathways leading to ERK1/2 activation results in cell-cycle exit and differentiation.

Calcium (Ca2+) has long been implicated as a regulator of mammalian muscle fusion; transient Ca2+ depletion from the sarcoplasmic reticulum (SR) is associated with myoblast differentiation and fusion. Moreover, the Ca2+-sensitive transcription factor, NFATc2, was reported to mediate myoblast recruitment and myotube expansion. Yet, the signaling cascades which lead to Ca2+ mediated myoblast fusion remain elusive. CaMKII is a member of the Ca2+/Calmodulin (CaM) dependent serine/threonine kinase family. CaMKII delta (δ) and gamma (γ), and to some extent beta (β) are the primary isoforms expressed in skeletal muscle. Upon Ca2+/CaM binding to individual subunits, cross-phosphorylation of neighboring subunits at T287 leads to a state of autonomous activation, by increasing the affinity for Ca2+/CaM several thousand-fold. Previously, CaMKII was identified for its role in Ca2+-dependent regulation of gene expression associated with muscle oxidative metabolism as well as components of the contractile machinery. However, to date, the role of CaMKII specifically as a mediator of the myoblast fusion has not been shown.

Additional background art includes U.S. Pat. No. 7,270,829, International Patent Application WO 2018/189738A1 (U.S. Publication No. 2020/100525A1), International Patent Application WO 2018/227016A1, International Patent Application WO 2017/124100A1, U.S. Patent Application Publication 2016/0227830A1, U.S. Patent Application Publication 20200165569, US Patent Application Publication 2020/0140821, US Patent Application Publication 2017/0218329, US Patent Application Publications 20200392461, 20200245658, 20200140810, 20200080050, 20160251625, 20190376026, 20210037870 and 20200140821. Relevant non-patent publications include Bunge, J., Wall Street Journal, Mar. 15, 2017 (2017-03-15); Hong, Tae Kyung et al, Food Science of Animal Resources, 41:355-372, 2021 and Michailovici, I. et al, Development 141:2611-2620, 2014.

### SUMMARY OF THE INVENTION

According to an aspect of some embodiments of the present invention there is provided a method of inducing multinucleated myotube formation, the method comprising contacting myogenic precursor cells from a farmed animal with an Extracellular Regulated Signaling Kinase (ERK1/2) inhibitor and/or an upregulator of intracellular Ca 2+.

According to an aspect of some embodiments of the present invention there is provided a method of inducing multinucleated myotube formation, the method comprising contacting myogenic precursor cells from a farmed animal with at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist, a calcium ionophore and a Calmodulin-dependent Protein Kinase II (CaMKII) activator.

According to some embodiments of the invention, the ERK1/2 inhibitor is selected from the group consisting of MK-8353 (SCH900353), SCH772984, CC-90003, Corynoxeine, ERK1/2 inhibitor 1, magnolin, ERK INT-1, ERK IN-2, ERK IN-3, LY3214996, Ravoxertinib, Ravoxertinib hydrochloride, VX-11e, FR 180204, Ulixertinib, Ulixertinib hydrochloride, ADZ0364, K0947, FRI-20 (ON-01060), Bromacetoxycalcidiol (B3CD), BVD523, DEL22379, FR180204, GDC0994, K0947, AEZ-131 (AEZS-131), AEZS-136, AZ-13767370, BL-EI-001, LTT, ASTX-029, TCS ERK 11e and CAY10561.

According to some embodiments of the invention, the MEK1 inhibitor is selected from the group consisting of Trametinib, PD98059, U0126 (U0126-EtOH), PD0325901, Selumetinib (AZD6244), Cobimetinib (GDC-0973, RG7420), Binimetinib (MEK162), CI-1040 (PD 184352), Refametinib (BAY 869766; RDEA119), Pimasertib (AS703026), Selumetinib (AZD6244), Cobimetinib hemifumarate, GDC-0623 (RG 7421), R04987655, AZD8330, (ARRY-424704), SL327, MEK inhibitor, PD318088, Cobimetinib racemate (GDC-0973 racemate; XL518 racemate) and EBI-1051.

According to some embodiments of the invention, the FGF inhibitor is selected from the group consisting of Derazantinib, PD 161570, SSR 128129E, CH5183284, PD 166866 and Pemigatinib.

According to some embodiments of the invention, the TGF-beta inhibitor is selected from the group consisting of SD208, LY364947, RepSox, SB 525334, R 268712 and GW 788388.

According to some embodiments of the invention, the RXR/RAR agonist is selected from the group consisting of CD3254, Docosahexaenoic acid, LG100268, SR11237, AC261066, AC55649, Adapalene, BMS961, CD1530, CD2314, CD437, BMS453, EC23, all-trans retinoic acid, all-trans-4-hydroxy retinoic acid, all-trans retinoic acid-d5, cyantraniliprole, Vitamin A, all-trans retinol, LG100754, Beta Carotene, beta-apo-13 carotene, lycopene, all-trans-5,6-epoxy retinoic acid, all-transe-13,14-Dihydroretinol, Retinyl Acetate, Hanokiol, Valerenic acid, HX630, HX600, LG101506, 9cUAB30, AGN194204, LG101305, UVI3003, Net-41B, CBt-PMN, XCT0135908, PA024, methoprene acid, 9-cis retinoic acid, AM80, AM580, and CH55, TTNPB, and Fenretinide, LG-100064, Fluorobexarotene (compound 20), Bexarotene (LGD1069), Bexarotene D4, NBD-125 (B-12), LGD1069 D4 and 9-cis-Retinoic acid (ALRT1057).

According to some embodiments of the invention, the RYR1, RYR3 agonist is selected from the group consisting of Caffeine, Chlorocresol, CHEBI:67113, chlorantraniliprole, S107hydrochloride, JTV519, Trifluoperazine (T FP), Xanthines, Suramin, Suramin sodium, NAADP tetrasodium salt, S100A1, Cyclic ADP-Ribose (ammonium salt), pentifylline, 4-chloro-3-methylphenol (4-chloro-m-cresol), tetraniliprole, trifluoperazine (TFP), cyclaniliprole and Cyantraniliprole.

According to some embodiments of the invention, the upregulator of intracellular Ca2+ is selected from the group consisting of NAADP tetrasodium salt, Cyclic ADP-Ribose, 4-bromo A23187, Ionomycin, A23187 and isoproterenol.

According to some embodiments of the invention, the CaMKII agonist is selected from the group consisting of Calcium, Calmodulin, CALP1 and CALP3.

According to some embodiments of the invention, the myogenic precursor cells are selected from the group consisting of myoblasts, satellite cells, muscle side population (mSP) cells, muscle-derived stem cells (MDSCs), mesenchymal stem cells (MSCs), muscle-derived pericytes, embryonic stem cells (ESCs), induced muscle progenitor cells (iMPCs) and Induced Pluripotent Stem cells (iPSCs).

According to some embodiments of the invention, the myogenic precursor cells express MyoD, Pax3 and Pax7, or the corresponding orthologs thereof.

According to some embodiments of the invention, the myogenic precursor cells are myoblasts.

According to some embodiments of the invention, the myogenic precursor cells are from a biopsy of said farmed animal.

According to some embodiments of the invention, the biopsy is a muscle biopsy.

According to some embodiments of the invention, the myogenic precursor cells are isolated from the biopsy by enzymatic dissociation and/or mechanical dissociation.

According to some embodiments of the invention, the myogenic progenitor cells are undifferentiated myogenic precursor cells cultured in proliferation medium prior to inducing multinucleated myotube formation.

According to some embodiments of the invention, the proliferation medium is devoid of molecules selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist, calcium ionophore and a Calmodulin-dependent Protein Kinase II (CaMKII) activator.

According to some embodiments of the invention, the myogenic progenitor cells are myogenic precursor cells cultured in a differentiation medium prior to inducing multinucleated myotube formation.

According to some embodiments of the invention, the culturing is effected in a single vessel.

According to some embodiments of the invention, the method of the invention is effected by supplementing said medium with any of said molecules.

According to some embodiments of the invention, the method is effected in the presence of serum or serum replacement at an amount which allows cell proliferation and/or under normoxic conditions.

According to some embodiments of the invention, the farmed animals are selected from the group consisting of mammals, birds, fish, invertebrates, reptiles and amphibians.

According to some embodiments of the invention, the multinucleated myotubes comprise at least three nuclei.

According to some embodiments of the invention, the multinucleated myotubes comprise at least ten nuclei.

According to some embodiments of the invention, the multinucleated myotubes express myogenic differentiation and fusion factors selected from the group consisting of MyoD, MyoG, Mymk and Mymx.

According to some embodiments of the invention, inducing multinucleated myotubes results in increased fraction of MYOG-positive nuclei, as compared to nuclei of myogenic progenitor cells cultured in differentiation medium without said at least one molecule.

According to some embodiments of the invention, inducing multinucleated myotube formation results in classical ladder-like striation of actinin and troponin signals and/or phalloidin staining representing actin filaments.

According to some embodiments of the invention, the multinucleated myotube formation comprises mononucleated myoblast-myotube fusion and/or expansion of bi- and tri-nucleated myotubes into large multinucleated fibers.

According to some embodiments of the invention, contacting the myogenic precursor cells is effected for 12-48 hours.

According to some embodiments of the invention, contacting the myogenic precursor cells is effected for 16-24 hours.

According to an aspect of some embodiments of the present invention there is provided a cultured meat composition comprising multinucleated myotubes produced by the methods of the invention.

According to an aspect of some embodiments of the present invention there is provided a comestible comprising the cultured meat composition of the invention.

According to some embodiments of the invention, the comestible is processed to impart an organoleptic sensation and texture of meat.

According to some embodiments of the invention, the comestible further comprises plant- and/or animal-originated foodstuffs.

According to some embodiments of the invention, the comestible further comprises adipocytes, muscle cells, blood cells, cartilage cells, bone cells, connective tissue cells, fibroblasts and/or cardiomyocytes.

According to some embodiments of the invention, the comestible of the invention, further comprises plant based protein.

According to an aspect of some embodiments of the present invention there is provided a method of producing food, the method comprising combining the cultured meat composition or the comestible of the invention with an edible composition for human or animal consumption.

According to an aspect of some embodiments of the present invention there is provided a method of treating a muscle injury in a farmed animal, the method comprising contacting injured muscle tissue with at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator, thereby inducing myotube regeneration and treating said muscle injury.

According to an aspect of some embodiments of the present invention there is provided at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator, for use in inducing myotube regeneration and treating a muscle injury in a farmed animal.

According to an aspect of some embodiments of the present invention there is provided a cell culture medium for preparing multinucleated myotubes from myogenic precursor cells, the culture medium comprising a base medium and an Extracellular Regulated Signaling Kinase (ERK1/2) inhibitor.

According to some embodiments of the invention the cell culture medium further comprises at least one of a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist, calcium ionophore and a Calmodulin-dependent Protein Kinase II (CaMKII) activator.

According to some embodiments of the invention the cell culture medium consisting of ingredients certified Generally Regarded As Safe (GRAS).

According to some embodiments of the invention the cell culture medium is a serum-free medium.

According to some embodiments of the invention the cell culture medium comprises a serum replacement ingredient.

According to some embodiments of the invention the cell culture medium consists of ingredients certified xeno-free.

According to an aspect of some embodiments of the present invention there is provided a method of inducing multinucleated myotube formation, the method comprising contacting myogenic precursor cells from a farmed animal with an Extracellular Regulated Signaling Kinase (ERK1/2) inhibitor and/or an upregulator of intracellular Ca 2+, wherein when the myogenic precursor cells are chicken myogenic precursor cells the contacting is performed in the presence of Extracellular Regulated Signaling Kinase (ERK1/2) inhibitor and an upregulator of intracellular Ca 2+.

According to an aspect of some embodiments of the present invention there is provided a method of inducing multinucleated myotube formation, the method comprising contacting myogenic precursor cells from a farmed animal with at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator wherein when the myogenic precursor cells are chicken myogenic precursor cells the contacting is performed in the presence of Extracellular Regulated Signaling Kinase (ERK1/2) inhibitor and an upregulator of intracellular Ca 2+.

According to some embodiments of the invention, the ERK1/2 inhibitor is selected from the group consisting of MK-8353 (SCH900353), CC-90003, Corynoxeine, ERK1/2 inhibitor 1, magnolin, ERK IN-1, ERK IN-2, ERK IN-3, LY3214996, Ravoxertinib, Ravoxertinib hydrochloride, VX-11e, FR 180204, Ulixertinib, Ulixertinib hydrochloride, ADZ0364, K0947, FRI-20 (ON-01060), Bromacetoxycalcidiol (B3CD), AEZ-131(AEZS-131), AEZS-136, AZ-13767370, BL-EI-001, LTT, Peptide inhibitors EPE, ERK Activation Inhibitor Peptide I (ERK inhibitor IV), ERK Activation Inhibitor Peptide II (ERK inhibitor V).

According to some embodiments of the invention, the MEK1 inhibitor is selected from the group consisting of Trametinib, PD98059, U0126 (U0126-EtOH), PD0325901, Selumetinib (AZD6244), Cobimetinib (GDC-0973, RG7420), Binimetinib (MEK162), CI-1040 (PD 184352), Refametinib (BAY 869766; RDEA119), Pimasertib (AS703026), Selumetinib (AZD6244), Cobimetinib hemifumarate, GDC-0623 (RG 7421), R04987655, AZD8330, (ARRY-424704), SL327, MEK inhibitor, PD318088, Cobimetinib racemate (GDC-0973 racemate; XL518 racemate) and EBI-1051.

According to some embodiments of the invention, the FGF inhibitor is selected from the group consisting of Derazantinib, PD 161570, SSR 128129E, CH5183284, PD 166866 and Pemigatinib.

According to some embodiments of the invention, the TGF-beta inhibitor is selected from the group consisting of SD208, LY364947, RepSox, SB 525334, R 268712 and GW 788388.

According to some embodiments of the invention, the RXR agonist is selected from the group consisting of CD3254, LG100268, LG-100064, SR11237 (BMS-649), Fluorobexarotene (compound 20), AGN194204 (IRX4204), Bexarotene (LGD1069), NBD-125 (B-12), Bexarotene D4, LGD1069 D4 and 9-cis-Retinoic acid (ALRT1057).

According to some embodiments of the invention, the RYR1, RYR3 agonist is selected from the group consisting of Chlorocresol, CHEBI:67113-chlorantraniliprole, S107 hydrochloride, JTV519, Trifluoperazine (TFP), Xanthines, Suramin, NAADP tetrasodium salt, S100A1, Cyclic ADP-Ribose (ammonium salt) and Cyantraniliprole.

According to some embodiments of the invention, the upregulator of intracellular Ca2+ is selected from the group consisting of NAADP tetrasodium salt, Cyclic ADP-Ribose, 4-bromo A23187, Ionomycin, A23187 and isoproterenol.

According to some embodiments of the invention, the CaMKII agonist is selected from the group consisting of Calcium, Calmodulin, CALP1 and CALP3.

According to some embodiments of the invention, the myogenic precursor cells are selected from the group consisting of myoblasts, satellite cells, muscle side population (mSP) cells, muscle-derived stem cells (MDSCs), mesenchymal stem cells (MSCs), muscle-derived pericytes, embryonic stem cells (ESCs) and Induced Pluripotent Stem cells (iPSCs).

According to some embodiments of the invention, the myogenic precursor cells are myoblasts.

According to some embodiments of the invention, the myogenic precursor cells are from a biopsy of the farmed animal.

According to some embodiments of the invention, the biopsy is a muscle biopsy.

According to some embodiments of the invention, the myogenic precursor cells are isolated from the biopsy by enzymatic dissociation and/or mechanical dissociation.

According to some embodiments of the invention, the myogenic progenitor cells are undifferentiated myogenic precursor cells cultured in proliferation medium prior to inducing the multinucleated myotube formation.

According to some embodiments of the invention, the proliferation medium is devoid of molecules selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator.

According to some embodiments of the invention, the method is effected in the presence of serum or serum replacement at an amount which allows cell proliferation and/or under normoxic conditions.

According to some embodiments of the invention, the farmed animals are selected from the group consisting of mammals, birds, fish, invertebrates, reptiles and amphibians.

According to some embodiments of the invention, the multinucleated myotubes comprise at least three nuclei.

According to some embodiments of the invention, the multinucleated myotubes comprise at least 10 nuclei.

According to some embodiments of the invention, the multinucleated myotubes express myogenic differentiation and fusion factors selected from the group consisting of MyoD, MyoG, Mymk and Mymx.

According to some embodiments of the invention, the inducing multinucleated myotubes results in increased fraction of MYOG-positive nuclei, as compared to nuclei of myogenic progenitor cells cultured in differentiation medium without the at least one molecule.

According to some embodiments of the invention, the multinucleated myotube formation is evident by classical ladder-like striation of actinin and troponin signals and/or phalloidin staining representing actin filaments.

According to some embodiments of the invention, a yield of myotube is higher than that obtained by incubating the myogenic precursor cells with DMEM 2% Horse Serum (HS) with 1% Pen/Strep (DM), as evident by any of fibers surface coverage, cell weight and amount of protein, as can be determined by Bradford.

According to some embodiments of the invention, the multinucleated myotube formation comprises mononucleated myoblast-myotube fusion and/or expansion of bi- and tri-nucleated myotubes into large multinucleated fibers.

According to some embodiments of the invention, the contacting the myogenic precursor cells is effected for 12-48 hours.

According to some embodiments of the invention, the contacting the myogenic precursor cells is effected for 16-24 hours.

According to an aspect of some embodiments of the present invention there is provided a cultured meat composition comprising multinucleated myotubes produced by the methods of the invention.

According to an aspect of some embodiments of the present invention there is provided a comestible comprising the cultured meat composition of the invention.

According to some embodiments of the invention, the comestible of the invention is processed to impart an organoleptic sensation and texture of meat.

According to some embodiments of the invention, the comestible of the invention further comprises plant- and/or animal-originated foodstuffs.

According to some embodiments of the invention, the comestible of the invention further comprises adipocytes, muscle cells, blood cells, cartilage cells, bone cells, connective tissue cells, fibroblasts and/or cardiomyocytes.

According to some embodiments of the invention, the comestible of the invention further comprises plant-based protein.

According to an aspect of some embodiments of the present invention there is provided a method of producing food, the method comprising combining the cultured meat composition of the invention or the comestible of the invention with an edible composition for human or animal consumption.

According to an aspect of some embodiments of the present invention there is provided a method of treating a muscle injury in a farmed animal, the method comprising contacting injured muscle tissue with at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator, thereby inducing myotube regeneration and treating the muscle injury, wherein when the myogenic precursor cells are of chicken the contacting is performed in the presence of Extracellular Regulated Signaling Kinase (ERK1/2) inhibitor and an upregulator of intracellular Ca 2+.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1I are a series of images and graphs showing induction of myoblast differentiation and hyper-fusion by ERK1/2 inhibition.
(1A) Representative images of myoblasts at different timepoints following treatment with a DMSO control (Ctrl) or 1 μM SCH772984 (ERKi) in growth medium, or Differentiation Medium (DM). Cells were fixed and stained at 8, 24, and 48 hours after treatment with the differentiation marker Myosin Heavy Chain (MyHC, red), and the nuclear Hoechst (blue). Scale bar=200 μm.
(1B) Fusion index representing the fraction of nuclei found in differentiated (MyHC⁺) cells in 1A. The total number of nuclei assayed, n=88,518. (1C) Representative qRT-PCR results showing the temporal gene expression profiles of Myod, Myog, Mymk, and Mymx during myogenesis. Gene expression values were normalized to Gapdh and expressed as fold change from the control at 0-hours. (1D, 1F, 1H) Representative images of myoblasts treated with DMSO Ctrl or 1 μM ERKi in growth medium, or DM for 24 hours and stained for MyHC (red), and MYOG (green) (1D), MyHC (red) and Ki-67 (green) (1F) and MyHC (red) and pH3 (green). Nuclei are stained with DAPI (blue). Scale bar=100 μm. (1D, 1E, 1G). The percent of MYOG, Ki-67 and PH3 respectively. All data are representative of at least 3 biological repeats. Error bars indicate SEM.;

FIGS. 2A-2J are a series of images and graphs showing that ERK1/2 inhibition initiates an RXR/RYR-dependent fusion response.
(2A) Co-immunoprecipitation of ERK1/2 and RXR. (2B) Representative images of cells treated with Ctrl, 1 μM ERKi, 20 μM HX531(RXRi), ERKi and RXRi, 50 μM Dantrolene (RYRi), ERKi and RYRi, 10 uM BAPTA-AM, or ERKi and BAPTA-AM at 24 hrs, and stained for the differentiation markers MyHC (red), MYOG (green) and nuclei (blue). White boxes indicate the portion of the field shown enlarged on the right. (2C) Fusion index for ERKi and RXRi co-treatment experiment. (2D) Quantification of MYOG positive nuclei per field for ERKi and RXRi co-treatment experiment. Total number of nuclei assayed for 2C and 2D, n=106,116. (2E) qRT-PCR analysis of the fold change in expression of calcium channels and sensors in vehicle (Ctrl) compared to cells treated with ERKi for 24 hrs; gene expression was normalized to Hprt. Values are expressed as fold change from that of Ctrl. (2F) qRT-PCR analysis of RYR1/3 gene expression demonstrates regulation by ERK1/2 and RXR. (2G) Fusion index for ERKi and RYRi co-treatment experiment. (2H) Quantification of MYOG positive nuclei per field for ERKi and RYRi co-treatment experiment. Total number of nuclei assayed for 2G and 2H, n=113,448.
(2I) Fusion index for ERKi and BAPTA-AM co-treatment experiment. (2J) Quantification MYOG positive nuclei per field for ERKi and BAPTA-AM co-treatment experiment. Total number of nuclei assayed for 2I and 2J, n=109,360. All data are representative of 3 biological repeats. Scale bars=100 μm;

FIGS. 3A-3M are a series of images and graphs showing that asymmetric myoblast fusion requires calcium-dependent CaMKII activation.
(3A) Representative western blots of CaMKII activation upon 24 hours treatment with ERKi or DM. (3B, 3C, and 3D) Representative western blots showing CaMKII activation in myoblasts following 24 hours treatment. Respectively, (3B) treatments were DMSO (Ctrl), 1 μM ERKi, 20 μM HX531(RXRi), or cotreated with ERKi and RXRi. (3C) treatments were Ctrl, 1 μM ERKi, Dantrolene 50 μM (RYRi), or cotreated with ERKi and RYRi. (3D) Treatments were Ctrl, 1 μM ERKi, 10 uM BAPTA-AM, or cotreated with ERKi and BAPTA-AM. (3E) Representative images immunofluorescent images of cells treated with Ctrl, 1 μM (ERKi), 5 μM KN93 (CaMKIIi), or co-treated with ERKi and CaMKIIi at 24 hrs. Cells were stained for the differentiation markers MyHC (red), MYOG (green) and DAPI (blue). Indicated regions are enlarged to the right. (3F) Fusion index for 3E; values are stratified by number of nuclei per MyHC+ fiber. Total number of nuclei assayed n=61,510. (3G) Quantification of MYOG positive nuclei per field of 3E. Total number of nuclei assayed n=112,901. (3H) qRT-PCR gene expression analysis of the experiment shown in 3D; gene expression was normalized to Hprt. Values are expressed as fold change from that of Ctrl. (3I) Representative western blot of CaMKII activation from gain/loss of function study with wildtype CaMKII (Ad-CaMKII^{WT}) or a phospho-null mutant (Ad-CaMKII T287) at 72 hours post transfer to in DM. Bands for endogenous and exogenous CaMKII are indicated. (3J) Quantification of the number of nuclei per MyHC⁺ cell for CaMKII gain/loss of function study at 72 hours treatment in DM, presented as fold change from control virus. Total number of nuclei assayed n=18,758. (3K) Representative western blot of time-course following treatment with 1 μM ERKi. (3L) Representative images showing Ryanodine receptor (RYR) localization in Ctrl and ERKi treated myofibers. Indicated region in ERKi image is enlarged on right showing the individual fluorescence channels and an overlay. Arrows indicate differentiated (MyHC⁺) myocytes lacking ryanodine receptor. (3M) Representative immunofluorescence staining showing p-CaMKII localization (green) primarily to myotubes, at 24 hours post treatment with ERKi. Indicated region in the ERKi image is enlarged on right showing the individual fluorescence channels and an overlay. Arrows indicate mononucleated MyHC+ cells which are negative for p-CaMKII, while the asterisk shows a binucleated MyHC+ cell which is p-CaMKII+. Arrowhead shows a MyHC+ cell which has already fused with a myotube and is p-CaMKII+. All data are representative of at least 3 biological repeats. Error bars indicate SEM. All scale bars=100 μm;

FIGS. 4A-4E are a series of images showing asymmetric myotube growth through recruitment of mono-nucleated myoblasts at fusogenic synapses.
(4A) hourly fusion index showing the distribution of mono-, bi-, tri- and multi-nucleated (n≥4) cells. At ˜16 hours post treatment with ERKi a marked increase in the number of multinucleated fibers is observed accompanied by a concomitant decrease in mono-nucleated cells. The average number of bi- and tri-nucleated cells remains relatively constant from −12 hours. Total number of nuclei assayed n=13,044. (4B) Data-driven simulations reveal that the fraction of nuclei in multinucleated (n: 4) cells is not recapitulated if fusion occurs with equal probability (inverted triangles) or with a weighted probability (upright triangles) considering that larger cells have a higher probability to fuse. The simulations were performed by estimating the number of fusion events for each hour in an experiment. The estimated number of fusions were used to simulate two simple scenarios: In random simulations, cells have a uniform probability of fusing, weighted simulations adjust the probability according to the number of nuclei in a cell. Statistical significance was determined with a bootstrapping approach, See Methods for full details. (4C) Frames acquired from time lapse microscopy of an individual myotube undergoing asymmetric fusion. At time 0 the bi-nucleated early myotube is seen labeled with a cytoplasmic DsRed (Purple) and approached by a mononucleated myoblast (yellow square) expressing a membrane targeted GFP (farnesylated-GFP; White). When the cells fuse, cytoplasmic and membrane mixing become apparent (t=00:28). Time: hh:mm. Scale bar: 50 μm (4D) Two examples of fusogenic synapses (Time: hh:mm). Scale bar is 10 μm. The “front view” represents the fusion event represented in 4C (yellow square). Top panel: Z projection of the membrane marker highlighting the 3D structure of the protrusion extending from the myoblast to the myotube where fusion eventually occurs as can be seen by the simultaneous diffusion of the cytoplasmic marker into the myoblast and the disappearance of the membrane marker from the protrusion between the two fusing cells. Middle panel: represents the specific Z plane of the membrane marker where the fusion pore can be seen expanding. Bottom panel: Z plane from the same time-lapse where a different fusion event is seen in a side view. Cyan and yellow arrows in the middle and bottom panels point to the fusogenic synapse before and after fusion, respectively. (4E) Frames acquired of GCaMP6S calcium reporter fluorescence in a growing myotube undergoing asymmetric fusion. Fluorescent signal is depicted as a heatmap. Solid arrow indicates a myotube about to recruit several myoblasts to fuse with it. Dashed arrow indicates one of these myoblasts prior to and during the first asymmetric fusion event. * at (00:10) indicates a calcium pulse in the growing myotube, which is absent in the myoblast. (Time scale: hh:mm). Scale bar=50 μm.

FIGS. 5A-5I are a series of images, graphs and blots showing that CaMKII is required for efficient muscle regeneration.
(5A) Western blot of analysis of indicated proteins from muscle following cardiotoxin (CTX)-induced muscle injuries. Line indicates where a lane was purposely removed. (5B) Schematic illustration of the satellite cell specific double CaMKII KO mouse model. (5C) Schematic illustration depicting the timeline of the repeat injury experimental design. (5D) Western blot validation of CaMKII depletion in WT or scDKO primary myoblasts isolated 2 weeks following initial injury. (5E) Immunofluorescence staining of WT or scDKO primary myoblasts following ERKi-induced fusion at 24 hrs post treatment. Insets are enlarged to the right. (5F) Fusion index comparison between WT (n=4) and scDKO (n=4) primary myoblasts stratified by number of nuclei per fiber. Total number of nuclei assayed n=12,743. (5G) Representative field of WT and scDKO muscle 14 days after CTX-induced reinjury. (5H) Quantification of myofiber cross sectional areas of WT (n=4) and scDKO (n=4) mice 14 days following reinjury. (SI) Average percentage of central nuclei in WT (n=4) and scDKO (n=4) mice 14 days following reinjury. At least 9,000 fibers per mouse were measured for 5H and 5I. Error bars indicate SEM. All scale bars, 100 μm;

FIGS. 6A-6C is a schematic representation of the ERK1/2-CaMKII myotube driven secondary fusion pathway.
Schematic of the ERK-CaMKII pathway during myoblast differentiation and fusion: 6A) In proliferating myoblasts ERK1/2 suppresses MYOG and p21/p27 activation. 6B) Upon ERK1/2 inhibition, p21/p27 are expressed and cells exit the cell cycle; simultaneously, MYOG is upregulated and cells become differentiated. 6C) During the differentiation process ERK1/2 inhibition results in transactivation of RXR leading to RYR1/3 upregulation and accumulation in the SR of early myotubes, eventually resulting in calcium-dependent CaMKII activation and CaMKII dependent myotube driven asymmetric fusion.

FIGS. 7A-7E are a series of images, blots and graphs showing the criticality of Ca-dependent CaMKII activation of multinucleate myotube development.
(7A) Representative western blot showing CaMKII activation of myoblasts treated with DMSO (Ctrl), 1 μM ERK inhibitor SCH772984 (ERKi), CaMKII inhibitor KN93 5 μM (CaMKIIi), or cotreated with ERKi and CaMKIIi at 24 hrs post treatment. (7B) Quantification of pH3 positivity following treatment with DMSO (Ctrl), 1 μM SCH772984 (ERKi), KN93 5 μM (CaMKIIi), or cotreated with ERKi and CaMKIIi at 24 hrs post treatment (7C) Quantification of cell motility of myoblasts treated with DMSO (Ctrl), 1 μM SCH772984 (ERKi), KN93 5 μM (CaMKIIi), or cotreated with ERKi and CaMKIIi over a 24-hour period. (7D) Representative IF images of myoblasts infected with control virus or virus expressing Myomaker, and treated with DMSO (Ctrl), 1 μM SCH772984 (ERKi), KN93 5 μM (CaMKIIi), or cotreated with ERKi and CaMKIIi for 18 hours. (7E) Quantification of the average number of nuclei per MyHC+ cell from 7D. All data are representative of at least 3 biological repeats. Error bars represent SEM.

FIG. 8 is the evaluation of the gene expression of several maturation markers in mouse myoblasts treated with SCH772984 compared to conventional differentiation media at 24 hours post treatment. qRT-PCR analysis of gene expression of Myh1, Myh2, and Tnnt3 was compared between myoblasts grown in proliferation media (CTRL), treated with μM ERK inhibitor SCH772984 (ERKi), or conventional differentiation media (DM). Gene expression is normalized to internal house keeping gene Hprt, and shown as fold change from CTRL.

FIGS. 9A-9C show that ERK inhibition induces a hyper differentiation and fusion phenotype in chicken myoblasts. (9A) Time-course experiment in chicken derived primary myoblasts demonstrating the effectiveness of ERKi treatment (1 μM SCH772984, ERKi) in proliferation media compared to conventional differentiation media (DM). Muscle fibers are indicated by staining for myosin heavy chain (Red) and nuclei are stained for DAPI (blue). (9B) A fusion index was quantified at 72 hours post treatment demonstrating a nearly 4× increase in fusion of myoblasts upon treatment with ERKi compared to DM. (9C) qRT-PCR analysis of the gene expression of various markers of differentiation throughout a 72 hour timecourse demonstrating that both ERKi treatment and DM induce differentiation, yet the effect of ERKi is more dramatic than that of DM.

FIGS. 10A-10B show that ERKi induces a more robust induction of chicken muscle fiber differentiation compared to conventional DM. (10A) qRT-PCR analysis of the gene expression of the transcription factor mrf4 and sarcomeric genes myosin heavy chains (myh1, myh2) and troponin (tnnt3) demonstrates significantly elevated expression following treatment with ERKi compared to DM. (10B) Immunoflourescent staining of ERKi treated chicken myoblasts at 48 hours post treatment for sarcomeric proteins including alpha-actinin, filamentous actin (phalloidin) and troponinT demonstrating the classical striation of mature sarcomere. No comparison can be made to DM fibers at this timepoint as they had not yet formed (attesting to the early phenotype obtained by ERKi).

FIGS. 11A-11D show a quantitative analysis of ERKi impact on yield of muscle tissue. (11A). ERKi treated fibers cover significantly more surface area compared to fibers induced in DM. (11B) Evaluation of the relative mass of the muscle product at 72 hours post-treatment with 1 μM SCH772984 (ERKi) compared to DM. Briefly, identical number of cells were treated with either condition. Following 72 hours, tissue culture plates were scraped and cells were collected and centrifuged. Wet weight of the pellet was measured. ERKi treatment results in approximately 40% increase in product mass at 72 hours post treatment. (11C) The number of starting cells needed to reach a final product of 1 kilogram at 72 hours post treatment with ERKi or DM was determined based ion the cell pellet data from 11A. (11D) The relative protein yield of the product of ERKi or DM treatment was determined at 72 hours post-treatment, demonstrating that ERKi induced myogenesis results in 4-fold increase in total protein yield.

FIG. 12 shows a conserved phenotype achieved upon ERKi treatment in bovine myoblasts compared to conventional differentiation medium. Immunoflourescence images and quantification of fusion index for bovine derived myoblasts following 72 hours of treatment in proliferation medium (PM), Differentiation medium (DM) or treatment with 0.5 uM SCH 772984 (ERKi). ERKi results in nearly 8-fold increase in fusion compared to DM.

FIG. 13 demonstrates that ERKi induced bovine myotubes show earlier maturation compared to those derived by treatment with DM. Shown is immunofluorescence staining of the sarcomeric components of myosin heavy chain (MyHC), alpha-actinin, and Tropoinin T at 96 hours post treatment either with proliferation media (PM), differentiation media (DM), or with 1 uM SCH 772984 (ERKi). Despite the presence of myotubes under treatment with DM at 96 hours, ERKi induced myotubes have significantly higher levels of these sarcomeric markers as demonstrated by quantification of the relative intensity of the fluorescent signal.

FIGS. 14A and 14B are a series of images and graphs showing the induction of robust myoblast fusion by multiple ERK inhibitors. Representative images (FIG. 14A) and fusion indexes (FIG. 14B) of primary bovine myoblasts treated with ERK inhibitors SCH772984, AZD0364, BVD523, DEL22379, FR180204, GDC0994, K0947, and LY3214996 (all at 1 uM) in proliferation media show similar levels of myoblast differentiation and fusion for all the ERK inhibitors. Samples were fixed at 72 hours after treatment and immunostained for sarcomeric alpha-actinin (red) and nuclei were stained with DAPI (cyan). Error bars represent SEM. Scale bars are 100 um.

FIGS. 15A and 15B are a series of images and graphs showing the effect of calcium ionophores on ERK-inhibitor-induced myoblast fusion. Representative images (FIG. 15A) and fusion indexes (FIG. 15B) of primary chicken myoblasts treated either with ERK inhibitor alone (SCH772984 1 uM, SCH) or in combination with various calcium ionophores (lonomycin-2 uM, and Calcymicin-1 uM, and Calcium ionophore I-2 uM) in proliferation media demonstrate the synergy of combined ERK inhibitor and calcium ionophore administration. Samples were fixed at 48 hours after treatment and immunostained for Myosin heavy chain (MF20, red) and nuclei were stained with DAPI (cyan). Error bars represent SEM. Scale bars are 100 um.

FIGS. 16A and 16B are a series of images and graphs showing the effect of Retinoid X receptor (RXR)/Ryanodine (RAR) agonists on ERK-inhibitor-induced myoblast fusion. Representative images (FIG. 16A) and fusion indexes (FIG. 16B) of primary chicken myoblasts treated either with ERK inhibitor alone (SCH772984 1 uM, SCH) or in combination with various RXR/RYR agonists (9-cis retinoic acid, 9-cis RA-200 nM, AM80-200 nM, AM580-100 nM, and CH55-200 nM, TTNPB 200 nM, and Fenretinide 200 nM) in proliferation media demonstrate the synergy of combined ERK inhibitor and RXR/RYR agonist administration. Samples were fixed at 48 hours after treatment and immunostained for Myosin heavy chain (MF20, red) and nuclei were stained with DAPI (cyan). Error bars represent SEM. Scale bars are 100 um.

FIGS. 17A and 17B are a series of images and graphs showing the effect of Ryanodine (RYR) agonists on ERK-inhibitor-induced myoblast fusion. Representative images (FIG. 17A) and fusion indexes (FIG. 17B) of primary chicken myoblasts treated either with ERK inhibitor alone (SCH772984 1 uM, SCH) or in combination with various RYR agonists (Caffeine −2 mM, and Suramin-10 μM) in proliferation media demonstrate the synergy of combined ERK inhibitor and RYR agonist administration. Samples were fixed at 48 hours after treatment and immunostained for Myosin heavy chain (MF20, red) and nuclei were stained with DAPI (cyan). Error bars represent SEM. Scale bars are 100 um.

FIGS. 18A and 18B are a series of images and graphs showing the superior effect of ERK inhibition compared to MEK inhibition on myoblast fusion phenotype. Representative images (FIG. 18A) and fusion indexes (FIG. 18B) of primary chicken myoblasts treated either with ERK inhibitor alone (SCH772984 1 or 10 uM) compared to myoblasts treated with MEK inhibitor (U0126 1 or 10 uM) in either proliferation medium (PM) or differentiation medium (DM) demonstrate the superior myoblast fusion achieved by ERK inhibition, in particular in the proliferation medium (PM). Samples were fixed at 48 hours after treatment and immunostained for Myosin heavy chain (MF20, red) and nuclei were stained with DAPI (cyan). Error bars represent SEM. Scale bars are 100 um.

### DESCRIPTION OF SPECIFIC EMBODIMENTS OF THE INVENTION

The present invention, in some embodiments thereof, relates to methods for differentiating myogenic progenitor cells and, more particularly, but not exclusively, to cultured meat and cultured meat products.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Current methods for culturing muscle cells for producing cultured meat (e.g. “in-vitro meat”, “lab meat”, “laboratory meat”) require a lengthy (up to 14 days for bovine species) differentiation step for myotube induction from expanded muscle stem/progenitor cells, increasing production cost and duration. The present inventors have uncovered methods for significantly enhancing the degree and rate of myoblast-multinucleate myotube transition, increasing efficiency and reducing cost of cultured meat production.

The present inventors have shown that cultured myogenic precursors can be induced to form large multinucleated myotubes by inhibition or reduction of ERK1/2 (see, for example FIGS. 1A, 1B), and that myogenic precursor-myotube transition, and asymmetrical fusion is associated with increased intracellular Ca 2+(see, for example, FIGS. 3E and 3F). Further, the present inventors have shown that enhancement of myoblast differentiation and fusion can be achieved with a variety of ERK inhibitors (Example 10), and that manipulation of factors downstream of ERK1/2, by Calcium ionophores (Example 11), RXR/RAR agonists (Example 12) and by RYR agonists (Example 13) can effectively augment the potency of ERK inhibition.

The present inventors demonstrate the superiority of ERK inhibition (ERKi) compared to conventional methods (referred to herein as “DM” in some embodiments of the invention) for the purposes of cultured meat. Specifically, as demonstrated on chicken myogenesis in tissue culture: ERKi strengthens the differentiation transcriptional program leading to earlier myotube initiation; ERKi enhances fusion leading to significantly larger myotubes; and ERKi enhances the maturation of myofibers through increased expression of maturation markers, leading to earlier formation of sarcomeric structures (see, for example, Example 7). Moreover, the present inventors demonstrate that the effect is conserved and evident in at least 2 more additional species, bovine and ovine. Similarly, data from bovine myoblasts demonstrates that ERKi induced fibers reach maturation faster than those achieved with DM. Taken together, the earlier differentiation and more robust fusion achieved by myoblast treatment with ERKi results in earlier maturation of myotubes ultimately contributing to increased production efficiency of cultured meat by increasing the of total mass of the meat product, area coverage, and finally increase in total protein yield.

Thus, in some embodiments, there is provided a method of inducing multinucleated myotube formation, the method comprising contacting myogenic precursor cells from a farmed animal with an Extracellular Regulated Signaling Kinase (ERK1/2) inhibitor and/or an upregulator of intracellular Ca 2+.

In other embodiments, there is provided a method of inducing multinucleated myotube formation, the method comprising contacting myogenic precursor cells from a farmed animal with an Extracellular Regulated Signaling Kinase (ERK1/2) inhibitor and/or an upregulator of intracellular Ca 2+, wherein when the myogenic precursor cells are of chicken the contacting is performed in the presence of Extracellular Regulated Signaling Kinase (ERK1/2) inhibitor and an upregulator of intracellular Ca 2+.

As used herein, the term “myogenic precursor” or “myogenic precursor cell” refers to any cell which can differentiate into a muscle cell. Myogenic precursors are critical for muscle regeneration. Although the most naturally abundant animal myogenic precursors are the satellite cells, which are found on the plasmalemmal surface of the muscle fiber, other cells with myogenic potential have been identified and may be suitable for use with the methods of the invention. These include mesodermally derived myoblasts, interstitially located muscle side population (mSP) cells, muscle derived stem cells (MDSC) and myo-endothelial cells from endothelial-associated myofibers, mesodermal pericytes and mesoangioblasts and mesodermal CD133+ progenitors.

The different myogenic precursor cells may be characterized by cellular marker profiles, for example, MyoD+ and Desmin+ for myoblasts, CD34+/−, Ckit- and CD45-for mSPs, CD56+ and CD29+ for muscle precursors, CD133+ and CD34+/− for CD133+ mesodermal progenitors.

As used herein, the term “multinucleated myotube” refers to fused myogenic precursors (e.g. fused myoblasts) having 3 or more nuclei. Mono- or bi-nucleated myogenic precursors, even if expressing myogenic differentiation markers, are not considered “multinucleated myotubes”.

As used herein, the term “multinucleated myotube” is equivalent to the terms “multinucleated myoblast”, “multinucleated muscle fibers”, “multinucleate muscle fibers”, “multinucleated syncitia”, “multinucleate syncitia”, “multinucleated muscle syncitium”, “multinucleate muscle syncitium”, “multinucleated muscle syncitium”, “multinucleate muscle syncitium”, and may be used interchangeably herein.

In some embodiments, the multinucleated myotubes have in the range of 4-10,000, 10-8,000, 20-500, 15-250, 50-1000, 100-800, 60-2000, 70-4000, 80-6000, 90-5000 nuclei per myotube. In specific embodiments, the multinucleated myotubes have between 10 and 100 between 10 and 500, or between 10 and 1000 nuclei. Thus, in some embodiments, the multinucleated myotubes comprise at least 3 nuclei, at least 10 nuclei, at least 50 nuclei or at least 100 nuclei.

Cell nuclei can be identified and quantified by a number of techniques, including, but not limited to immunofluorescence, flow cytometry and immunohistological techniques. Common nuclear stains include DAPI (fluorescent), hematoxylin (cytological stain), Hoechst 33258 and 33342 (fluorescent), methyl blue (cytological stain), safranin (cytological). In specific embodiments, the nuclei are labelled with either Hoechst 3342 (Thermo-Fisher) or DAPI (Sigma), and visualized by fluorescent microscopy. In some embodiments, multinucleated myotube formation is quantified by stratification of the cells into mono- and bi nucleated cells as opposed to the multinucleated myotubes with four (3) or more nuclei.

In addition to developing multiple nuclei, myogenic precursor cells induced to form multinucleated myotubes enlarge by fusion with differentiating myogenic cells. While reducing the invention to practice, the present inventors have shown that the myogenic precursor-myotube formation includes “asymmetric fusion”, that is, rather than enhanced fusion of myoblast to myoblast (“primary fusion”), fusion according to the methods of the present invention is predominately fusion of myoblast-to-myotube fusion (“secondary fusion”, “asymmetric fusion”). Thus, according to some embodiments of the invention, multinucleated myotube formation comprises mononucleated myoblast-myotube fusion and/or expansion of bi- and tri-nucleated myotubes into large multinucleated fibers.

Additionally, in some embodiments, the myogenic precursor cells can be embryonic stem cells (ESCs, totipotent cells) and Induced Pluripotent Stem Cells (iPSCs). iPSCs can be created by from adult fibroblasts by induced expression of reprogramming factors, have limitless replicative capacity in vitro and can differentiate into myoblast-like cells (see, for example, Roca et al, J. Clin. Med 2015).

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus, and cells originating from an unfertilized ova which are stimulated by parthenogenesis (parthenotes).

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics.

In some embodiments, the myogenic precursor cells can be induced muscle progenitor cells obtained by transdifferentiation of non-muscle tissue (e.g. fibroblasts) directly into muscle progenitors by manipulation of small molecules in the medium, and/or forced expression of MyoD in the non-muscle cells. US Patent Application No. 2019/061731 to Hochedlinger et al discloses methods for producing induced muscle progenitor cells (iMPCs) having a satellite cell phenotype from fibroblasts, without passage through the iPS cell stage. Bin Xu et al (Nature Research, Scientific Reports DOI: 10.1038/s41598-020-78987-8, 2020) discloses transdifferentiation of fibroblasts by forced induction of MyoD. As used herein, “transdifferentiation” refers to a process in which a somatic cell transforms into another somatic cell without undergoing an intermediate pluripotent state or progenitor cell type.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

Hematopoietic stem cells, which may also be referred to as adult tissue stem cells, include stem cells obtained from blood or bone marrow tissue of an individual at any age or from cord blood of a newborn individual. Placental and cord blood stem cells may also be referred to as “young stem cells”.

Mesenchymal stem cells are multipotent stromal cells that can differentiate into a variety of cell types, including osteoblasts (bone cells), chondrocytes (cartilage cells), myocytes (muscle cells) and adipocytes (fat cells which give rise to marrow adipose tissue). The term encompasses multipotent cells derived from the marrow as well as other non-marrow tissues, such as placenta, umbilical cord blood, adipose tissue, adult muscle, corneal stroma or the dental pulp of deciduous baby teeth. The cells do not have the capacity to reconstitute an entire organ.

The myogenic precursor cells can be freshly isolated cells, cells cultured in primary culture from live tissue, or cells of isolated myogenic cell lines developed from repeated serial passages of primary muscle cells. Exemplary animal cell lines suitable for foods containing cultured animal cells are disclosed US Patent Application Publication 2021/037870 to Kreiger, et al. In some embodiments, the myogenic precursor cells can be genetically modified, for example, for enhanced proliferation or for expression of tissue-specific factors (see, for example, US Patent Application Publication 2020/0140821 to Elfenbein et al).

According to some embodiments of the invention, when taken freshly from a tissue biopsy or a primary culture, an initial stage of enrichment for myoblasts is performed. Specifically, the cells are cultured on non-coated dishes which allow for preferential adherence of fibroblasts. Myoblasts which predominantly remain in the suspension are collected and plated again so as to remove the fibroblasts and obtain an enriched culture of myoblasts. This process is termed “preplating”. The process may be repeated as needed (e.g., 2-4 times). The presence of fibroblasts on the dish can be monitored by microscopy.

Thus, in some embodiments, the myogenic precursor cells are selected from the group consisting of myoblasts, satellite cells, muscle side population (mSP) cells, muscle-derived stem cells (MDSCs), mesenchymal stem cells (MSCs), muscle-derived pericytes, embryonic stem cells (ESCs) and Induced Pluripotent Stem cells (iPSCs).

Recent reports have shown the establishment of stem-cell lines from domesticated ungulate animals e.g. (Challenges and prospects for the establishment of embryonic stem cell lines of domesticated ungulates. Anim Reprod Sci. 2007; 98 (1-2):147-168. doi: 10.1016/j.anireprosci.2006.10.009., which is hereby incorporated by reference). Bach et al. (Engineering of muscle tissue. Clin Plast Surg. 2003; 30(4):589-599. doi: 10.1016/S0094-1298(03)00077-4.) suggested myosatellite cells as the preferred source of primary myoblasts because they recapitulate myogenesis more closely than immortal myogenic cell lines. Myosatellite cells have been isolated and characterized from the skeletal muscle tissue of cattle (Dodson et al. Optimization of bovine satellite cell derived myotube formation in vitro. Tissue Cell. 1987; 19(2):159-166. doi: 10.1016/0040-8166(87)90001-2), chicken (Yablonka-Reuveni et al. Dev Biol. 1987; 119(1):252-259. doi: 10.1016/0012-1606(87)90226-0), fish (Powell et al. Cultivation and differentiation of satellite cells from skeletal muscle of the rainbow trout Salmo gairdneri. J Exp Zool. 1989; 250(3):333-338), lambs (Dodson et al. Isolation of satellite cells from ovine skeletal muscles. J Tissue Cult Methods. 1986; 10(4):233-237. doi: 10.1007/BF01404483), pigs (Blanton Blanton et al. Isolation of two populations of myoblasts from porcine skeletal muscle. Muscle Nerve. 1999; 22(1):43-50. doi: 10.1002/(SICI)1097-4598(199901)22:1, Wilschut et al. Isolation and characterization of porcine adult muscle-derived progenitor cells. J Cell Biochem. 2008; 105(5):1228-1239), and turkeys (McFarland et al. Proliferation of the turkey myogenic satellite cell in a serum-free medium. Comp Biochem Physiol. 1991; 99 (1-2):163-167. doi: 10.1016/0300-9629(91)90252-8). Porcine muscle progenitor cells have the potential for multilineage differentiation into adipogenic, osteogenic and chondrogenic lineages, which may play a role in the development of co-cultures (Wilschut et al. 2008, supra).

Alternatively, as mentioned, adult stem cells from farmed animal species can be used. For instance, myosatellite cells are an adult stem-cell type with multilineage potential (Asakura et al. Differentiation. 2001; 68 (4-5):245-253. doi: 10.1046/j.1432-0436.2001.680412). These cells also have the capacity to differentiate into skeletal muscle cells. A rare population of multipotent cells found in adipose tissue known as adipose tissue-derived adult stem cells (ADSCs) is another relevant cell type for in vitro meat production (Gimble et al. Adipose-derived stem cells for regenerative medicine. Circ Res. 2007; 100(9):1249-1260. doi: 10.1161/01.RES.0000265074.83288.09) which can be obtained from subcutaneous fat and subsequently transdifferentiated to myogenic, osteogenic, chondrogenic or adipogenic cell lineages (Kim et al. Muscle regeneration by adipose tissue-derived adult stem cells attached to injectable PLGA spheres. Biochem Biophys Res Commun. 2006; 348(2):386-392. doi: 10.1016/j.bbrc 0.2006.07 0.063).

Matsumoto et al. (J Cell Physiol. 2007; 215(1):210-222.) reported that mature adipocytes can be dedifferentiated in vitro into a multipotent preadipocyte cell line known as dedifferentiated fat (DFAT) cells, reversion of a terminally differentiated cell into a multipotent cell type. These DFAT cells are capable of being transdifferentiated into skeletal myocytes (Kazama et al. Mature adipocyte-derived dedifferentiated fat cells can transdifferentiate into skeletal myocytes in vitro. Biochem Biophys Res Commun. 2008; 377(3):780-785. doi: 10.1016/j.bbrc.2008.10.046) and are an attractive alternative to the use of stem cells.

In specific embodiments, the myogenic precursors are myoblasts.

Myogenic precursors may be characterized by levels of expression of certain cellular markers, such as, but not limited to ATP binding cassette transporter G2 (ABCG2), MCadherin/Cadherin15, Caveolin-1, CD34, FoxK1, Integrin alpha7, Integrin alpha 7 beta 1, MYF-5, MyoD (MYF3), Myogenin (MYF4), neural cell adhesion molecule 1 [NCAM1 (CD56)], CD82, CD318 Pax3 and Pax7. In some embodiments, the myogenic precursor cells are cells expressing significant levels of at least one of MyoD, Pax3 and Pax7, or corresponding, species-appropriate orthologs thereof. In other specific embodiments, the myogenic precursor cells express MyoD and at least one of Pax3 and Pax7, or corresponding, species-appropriate orthologs thereof. In particular embodiments, the myogenic precursor cells express all of MyoD, Pax3 and Pax7 or corresponding, species-appropriate orthologs thereof.

Once the myogenic precursor cells are obtained, they can be grown in culture to expand their mass, then form multinucleated myotubes, which can be later be formed into a cultured meat composition. Culturing the cells includes providing a culture system, transferring basal medium or basal medium supplemented with serum, serum-replacement and/or growth factors and other components as might be needed for the efficient growth of cells, into culturing vessels, adding cells and culturing the cells. The basal medium (e.g. Dulbecco's Modified Eagle Medium; DMEM) may include water, salts, vitamins, minerals, amino acids and a carbon source such as glucose. In some embodiments of the invention, the basal medium includes animal-derived growth factors. In other embodiments, the basal medium includes non-animal-derived growth factors.

In some embodiments of the invention, the basal medium includes an animal derived serum. In other embodiment of the invention, the basal medium of the current invention does not include animal derived serum such as fetal bovine serum, calf serum or horse serum. As used herein, by “does not include animal serum” or “animal serum-free” is meant that the medium contains less than about 1% or less than about 0.5% or less than about 0.1% or less than about 0.01% or zero animal derived serum by total weight of the medium. It is envisioned within some embodiments of the invention that a serum-free medium may contain growth factors and other substances, but nothing derived from an animal.

According to some embodiments, culturing is effected in the presence of serum at a level which is not considered starvation conditions that prevent cell proliferation. For example, above 2% serum (e.g., 3-25%). According to some embodiments, the conditions comprise 5-25%, 10-25% serum e.g., 15-25% serum, about 20% serum. Such conditions are provided in the Examples section which follows. Thus, according to an embodiment, the medium is BIO-AMF™-2 medium (e.g., available from Biological Industries), which comprises a basal medium supplemented with fetal calf serum (FCS), steroids, basic fibroblast growth factor, insulin, glutamine, and antibiotics.

According to some embodiments, culture of myogenic precursors or progenitors, and culture of multinucleated myotubes is effected in medium having ingredients and components which are Generally Regarded As Safe (GRAS) and/or “xeno-free”. In some embodiments, the medium comprises ingredients and/or components certified GRAS and or xeno-free. In other embodiments, the medium comprises ingredients and/or components certified GRAS and xeno-free. In other embodiments, the medium consists of ingredients and/or components certified GRAS and/or xeno-free. In still other embodiments, the medium consists of ingredients and/or components certified GRAS and xeno-free.

A list of media components used in meat production along with their worst-case exposure estimates and relevant authoritative limits or published toxicological/safety data supporting their use is presented in Table 1.

TABLE 1
Exemplary List of Cell Culture Media Ingredients Used During Meat Production, Risk Categorization, and Safety Information

| | | | | Maximum | | |
|---|---|---|---|---|---|---|
| | | | Risk | Estimated | | Safe Reference Level |
| | | | Assessment | Exposure per | | for Human Dietary |
| Class | Compound | CAS # | Category | 100 g serving** | | Intake (e.g., UL, OSL) |
| Amino Acids | L-ALANINE | 56-41-7 | 2 | N/A* | | UL = N/A* |
| | L-ARGININE-HCL | 1119-34-2 | 2 | N/A* | | UL = N/A* |
| | L-ASPARAGINE-H20 | 5794-13-8 | 2 | N/A* | | UL = N/A* |
| | L-ASPARTIC ACID | 56-84-8 | 2 | N/A* | | UL = N/A* |
| | L-CYSTEINE HCL | 7048-04-06 | 2 | N/A* | | UL = N/A* |
| | H20 | | | | | |
| | L-CYSTINE 2HCL | 56-89-3 | 2 | N/A* | | UL = N/A* |
| | L-GLUTAMIC ACID | 6106-04-03 | 2 | N/A* | | UL = N/A* |
| | MONOSODIUM | | | | | |
| | H2O | | | | | |
| | L-GLUTAMINE | 56-85-9 | 2 | N/A* | | UL = N/A* |
| | GLYCINE | 56-40-6 | 2 | N/A* | | UL = N/A* |
| | L-HISTIDINE-HCL- | 5934-29-2 | 2 | N/A* | | UL = N/A* |
| | H20 | | | | | |
| | L-ISOLEUCINE | 73-32-5 | 2 | N/A* | | UL = N/A* |
| | L-LEUCINE | 61-90-5 | 2 | N/A* | | UL = N/A* |
| | L-LYSINE-HCL | 657-27-2 | 2 | N/A* | | UL = N/A* |
| | L-METHIONINE | 63-68-3 | 2 | N/A* | | UL = N/A* |
| | L-PHENYLALANINE | 63-91-2 | 2 | N/A* | | UL = N/A* |
| | L-PROLINE | 147-85-3 | 2 | N/A* | | UL = N/A* |
| | L-SERINE | 56-45-1 | 2 | N/A* | | UL = N/A* |
| | L-THREONINE | 72-19-5 | 2 | N/A* | | UL = N/A* |
| | L-TRYPTOPHAN | 73-22-3 | 2 | N/A* | | UL = N/A* |
| | L-TYROSINE-2NA- | 122666-87-9 | 2 | N/A* | | UL = N/A* |
| | 2H20 | | | | | |
| | L-VALINE | 72-18-4 | 2 | N/A* | | UL = N/A* |
| Carbon | D-GLUCOSE | 50-99-7 | 1 | N/A | | NS |
| substrates | SUCCINIC ACID | 110-15-6 | 2 | 200 | mg | Intakes for a 65 kg |
| | | | | | | adult are 257-fold |
| | | | | | | below NOAEL from 2 |
| | | | | | | year rat study |
| | SODIUM | 6106-21-4 | 2 | 40 | mg | Intakes for 65 kg adults |
| | SUCCINATE-2Na | | | | | are >1000 fold below |
| | 6H20 | | | | | NOAEL from 2 year rat |
| | | | | | | study. |
| | SODIUM PYRUVATE | 113-24-6 | 3 | 1 | g | Dietary intakes within |
| | | | | | | the range of OSL |
| | | | | | | valued reported in |
| | | | | | | human investigations |
| Vitamins | FOLIC ACID | 59-30-3 | 1 | 60 | mg | UL = 300 to 1000 ug/d |
| | | | | | | (upper limit not |
| | | | | | | established based on |
| | | | | | | toxicity, but to prevent |
| | | | | | | people who don't |
| | | | | | | know they have a |
| | | | | | | vitamin B deficiency |
| | | | | | | from consuming too |
| | | | | | | much folic acid) |
| | DL-ALPHA- | 7695-91-2 | 1 | 8 | mg | UL = 1000 mg/d |
| | TOCOPHEROL | | | | | |
| | ACETATE | | | | | |
| | VITAMIN D2 | 50-14-6 | 1 | 24 | ug | UL = 100 ug/d |
| | (ERGOCALCIFEROL) | | | | | |
| | D-ALPHA- | 59-02-9 | 1 | 1.6 | mg | UL = 1000 mg/d |
| | TOCOPHEROL | | | | | |
| | D-BIOTIN | 58-85-5 | 1 | 4 | mg | UL = N/A |
| | MYO-INOSITOL | 87-89-8 | 1 | 430 | mg | OSL = 4 g/d |
| | NIACINAMIDE | 98-92-0 | 1 | 25 | mg | UL = 10 to 35 mg |
| | (NICOTINAMIDE) | | | | | (supplements) |
| | PYRIDOXINE-HCL | 58-56-0 | 1 | 25 | mg | UL = 30 to 100 mg |
| | PYRIDOXAL-HCL | | 1 | | | UL = 30 to 100 mg |
| | RIBOFLAVIN | 83-88-5 | 1 | 3.7 | mg | UL = NS |
| | THIAMINE-HCL | 67-03-8 | 1 | 18 | mg | UL = NS |
| | VITAMIN B12 | 68-19-9 | 1 | 2 | mg | UL = NS |
| | (CYANCOCOBALAMIN) | | | | | |
| | CALCIUM D- | 0137-08-06 | 1 | 100 | mg | UL = NS |
| | PANTOTHENATE | | | | | |
| | CHOLINE CHLORIDE | 67-48-1 | 1 | 52 | mg | UL = 3.5 g/d |
| | ASCORBIC ACID | | 1 | 2 | g | UL = 400 to 2000 mg |
| | | | | | | (theoretical ascorbic |
| | | | | | | acid concentrations |
| | | | | | | represent gross over |
| | | | | | | estimates and likely |
| | | | | | | exceed concentrations |
| | | | | | | by an order of |
| | | | | | | magnitude and are |
| | | | | | | expected to be present |
| | | | | | | at levels well below |
| | | | | | | the UL). |
| Trace Metals | SODIUM CHLORIDE | 7647-14-5 | 1 | — | | 2.3 g/d |
| | CALCIUM | | 1 | 760 | mg | 1 to 3 g/d |
| | CHLORIDE (ANHY) | | | | | |
| | MANGANESE | 10034-96-5 | 1 | 3 | ug | 2 to 11 mg/d |
| | SULFATE H2O | | | | | |
| | POTASSIUM | 7447-40-7 | 1 | 4 | g | UL = NS |
| | CHLORIDE | | | | | |
| | MAGNESIUM | | 1 | 170 | mg | 65 to 350 mg |
| | CHLORIDE (ANHY) | | | | | |
| | COPPER SULFATE- | 7758-99-8 | 1 | 3 | mg | 1 to 10 mg |
| | PENTAHYDRATE | | | | | |
| | SODIUM | 13517-24-3 | 1 | 1.6 | mg | Intakes for a 65 kg |
| | METASILICATE- | | | | | adult are 31,000 fold |
| | 9H2O | | | | | below rat NOAEL |
| | SODIUM SELENITE | 10102-18-8 | 2 | 190 | ug | Intakes for 65 kg adult |
| | (ANHY) | | | | | are 133-fold below 13 |
| | | | | | | week rat NOAEL |
| | ZINC SULFATE-7H20 | 7446-20-0 | 1 | 15 | mg | UL = 5 to 40 mg/day |
| | MAGNESIUM | 7487-88-9 | 1 | 493 | mg | No UL for food sources; |
| | SULFATE (ANHY) | | | | | UL = 65 to 350 mg |
| | | | | | | (dietary supplements) |
| | FERRIC NITRATE- | 7782-61-8 | 3 | 0.3 | mg | Iron UL = 40 to 45 mg; |
| | 9H2O | | | | | Nitrate ADI = 3.7 |
| | | | | | | mg/kg bw/day (levels |
| | | | | | | of iron and nitrate are |
| | | | | | | orders of magnitude |
| | | | | | | below the UL and ADI) |
| | FERROUS SULFATE- | 7782-63-0 | 2 | 2.5 | mg | Iron UL = 40 to 45 mg |
| | 7H2O | | | | | |
| | FERRIC CITRATE | 3522-50-7 | 2 | (Present as a | | Iron UL = 40 to 45 mg |
| | | | | residue of seed- | | |
| | | | | train scale-up and | | |
| | | | | not used as | | |
| | | | | nutrient source of | | |
| | | | | iron) | | |
| | FERRIC | 1185-57-5 | 2 | (Present as a | | Iron UL = 40 to 45 mg |
| | AMMONIUM | | | residue of seed- | | |
| | CITRATE | | | train scale-up and | | |
| | | | | not used as a | | |
| | | | | nutrient source of | | |
| | | | | iron) | | |
| Iron Carrier | Bovine Transferrin | 11096-37-0 | 3 | 150 | mg | NR |
| | SODIUM | 10049-21-5 | 1 | 1.5 | g | NR |
| | PHOSPHATE | | | | | |
| | MONOBASIC H20 | | | | | |
| | SODIUM | 7558-79-4 | 1 | 4.3 | g | NR |
| | PHOSPHATE | | | | | |
| | DIBASIC-ANHY | | | | | |
| Supplemen- | LINOLEIC ACID | 60-33-3 | 2 | 1 | mg | AI = 7 to 12 g/d |
| tary Lipids | | | | | | |
| Emulsifier | MONOETHANOLAMINE | 141-43-5 | 2 | 160 | mg | Dietary intakes for 65 |
| | | | | | | kg adult are 128 fold |
| | | | | | | below rat NOAEL |
| Surfactant/ | TWEEN 80 | 9005-65-6 | 2 | 4 | mg | ADI = 10 mg/kg bw/d |
| Emulsifier | | | | | | |
| Antioxidant | L-GLUTATHIONE | 70-18-8 | 2 | 36 | mg | NR |
| Animal | CHICKEN SERUM | www.thermofisher.com/us/en/home/ | 3 | — | | NR |
| components | | life-science/cell-culture/ | | | | |
| | | mammalian-cell-culture/ | | | | |
| | | fbs/other-sera/chicken-serum.html | | | | |
| | BOVINE SERUM | www.thermofisher.com/order/catalog/product/ | 3 | — | | NR |
| | | 16170060?SID=srch-srp-16170060#/ | | | | |
| | | 16170060?SID=srch-srp-16170060 | | | | |
| | BOVINE SERUM | www.thermofisher.com/order/catalog/product/ | 3 | — | | NR |
| | ALBUMIN | 11020021#/11020021 | | | | |
| Hydrolysate | HYPEP 2 YEAST | 8013-01-02 | 2 | 2 | g | NR |
| | EXTRACT | | | | | |

| | | | | | |
|---|---|---|---|---|---|
| Growth | FGF2 (FIBROBLAST | N/A | 3 | — | NR |
| Factors | GROWTH FACTOR- | | | | |
| | BASIC) | | | | |
| | PDGF-BB (PLATELET | N/A | 3 | — | NR |
| | DERIVED GROWTH | | | | |
| | FACTOR-BB) | | | | |
| | IGF-1 (INSULIN-LIKE | N/A | 3 | — | NR |
| | GROWTH FACTOR 1) | | | | |
| Peptides | RGD | 99896-85-2 | | 0.3 ug (Present as | N/A |
| | | | | a residue of seed- | |
| | | | | train scale-up and | |
| | | | | is not directly | |
| | | | | added to meat | |
| | | | | production media) | |
| | YIGSR | 110590-64-2 | | 0.3 ug (Present as | |
| | | | | a residue of seed- | |
| | | | | train scale-up, not | |
| | | | | directly added to | |
| | | | | meat production | |
| | | | | media) | |

AI = Adequate Intake
ADI = Acceptable Daily Intake
FNB-IOM = Food and Nutrition Board of the Institute of Medicine
HDT = Highest Dose Tested
N/A = Not applicable
NR = Not reported
NS = not specified as no evidence of toxicity from excess intake known
NOAEL = No Observed Adverse Effect Level
UL = Tolerable Upper Limit
OSL = Observed Safe Level
*No upper limit when provided as dietary protein
**Maximum dietary exposures estimated using conservative assumption of complete transfer of media components to the finished product on a wt/wt basis.

Category 1: These cell culture media components are food ingredients/additives that are GRAS or permitted by federal regulation without limitation on use. Exemplary compounds in this category include innocuous ingredients such as sugars, pH buffers, water soluble vitamins, and common antioxidants such as tocopherols.

Category 2: These cell culture media components are common dietary nutrients and are anticipated to have GRAS status for food use or be permitted by regulation for addition to food. Examples of such compounds include most of the inorganic salts and macronutrients that are present within the cell culture media. Where these compounds are permitted for direct addition to food at use levels comparable to anticipated concentrations that might reasonably be expected in the cell-based meat product, no safety concerns are anticipated. Majority of nutrients present within the poultry cell-based meat may be readily measured using common validated methods for food composition testing. Batch analyses of multiple lots of the finished product may be obtained to validate the above assumptions. In some instances, consideration of established safe levels (e.g., ADI, UL) derived from a relevant authoritative body (e.g., U.S. FDA, EFSA, JECFA, FSANZ, U.S. EPA) may be leveraged to support safety. If comparisons of anticipated dietary intakes relative to an authoritative reference intake value is used, consideration of additive intakes from all dietary sources may be considered. In the absence of an authoritative reference intake value, published NOAELs from animal toxicology studies may be used to evaluate safety using standard scientific procedures for food safety evaluation. A margin of exposure (MoE) of 100-fold or greater between the NOAEL and estimated dietary intakes from food exposures is typically considered adequate to support safety. in situations where the MoE is <100-fold, additional measures for further reduction of the media component may be necessary, or further characterization of intraspecies/interspecies differences in metabolism may be necessary. These situations also require careful consideration of the regulatory status on a case-by basis (e.g., premarket approval as a food additive or GRAS evaluation required).

Category 3: These cell culture media components have not been previously used in food production (e.g., no federal regulations or previous GRAS status) but with sufficient information to conclude that the compounds do not present risk for intended use in food production. For example, situations where the compound is not detectable in finished product or is present at equivalent levels in comparator foods, compounds that are thermo-labile and will be digested during cooking, and/or compounds that are expected to be digested to innocuous compounds following ingestion. Examples of compounds meeting the aforementioned conditions would include recombinant growth factors and serum components. For Category 3 substances a final consideration in the safety assessment process may involve hazard characterization of the potential for a substance to produce toxic biological effects outside of the endpoints measured in a sub-chronic rat toxicity study. Substances with biological activity may require additional hazard characterization related to reproductive and developmental toxicity, or immunotoxicity. Considerations for allergenicity, biological effects in humans (e.g., effects on blood pressure), and synergistic effects with other media components also may be evaluated. Such investigations may preferably be evidence-based (i.e., availability of a clinical trial demonstrating that a substance affects blood pressure), rather than theoretical (i.e., based on presumptive mechanisms of action). Similar to category 2 substances, the regulatory status of ingredients in category 3 will require case-by-case evaluation of the regulatory status of the compound (e.g., need for premarket approval or GRAS evaluation). Examples of category 3 components include recombinant proteins and animal serum.

According to some embodiments, the ingredients of the culture medium are certified “Generally Regarded As Safe” (GRAS) ingredients (e.g. category 1 and some of category 2 of Table 1). As used herein, certification of GRAS status is conferred by a recognized regulatory agency such as the USFDA. FDA GRAS certification can be granted (or declined) either on the basis of the use in food prior to 1958, or, for other substances, on the basis of documentation of a safety analysis conducted by the manufacturer, and reviewed by the FDA. According to some embodiments, the ingredients of the culture medium are certified “xeno-free” ingredients. As used herein, the term “xeno-free” medium refers to a cell culture medium which is devoid of components originating from species other than those of the cultured cells. In some embodiments, the term “xeno-free” relates to “non-human-free”, or the absence of components originating from species other than humans.

Cells for expansion in the cell culture may be obtained by biopsy from a live farmed animal, for example, from fish, pig, cows, chicken, turkey, sheep, goat and the like.

As used herein, the term “farmed animal” refers to any animals which are grown (cultivated) for agricultural purposes, and, in particular, for provision of meat for consumption. Thus, farmed animals include, but are not limited to avian species, mammalian species, invertebrates (e.g. shellfish), reptiles (e.g. alligators, crocodiles, snakes, turtles, etc.) and amphibians (e.g. frogs). Farmed animals include domesticated species (e.g. cows, chickens, pigs, ducks, sheep, etc.) and non-domesticated species (trout, salmon, lobster, shrimp, etc.). Examples of avian species suitable for use with the methods of the invention include, but are not limited to geese, ducks, chicken, Cornish hen, pheasants, turkeys, Guinea hen, quails, partridge, pigeons, emu, ostrich, capons, grouse, swan, doves, woodcocks, chukars and snipes. Examples of farmed aquatic species suitable for use with the methods of the invention include, but are not limited to carp, tilapia, salmon, milkfish, trout, bream, snakehead, eel, catfish, rohu, halibut, seabass, cod, rabbitfish, shrimp, crayfish, prawns, lobster, crab, oyster and claims. Examples of farmed mammalian species suitable for use with the methods of the invention include, but are not limited to cattle, bison, buffalo, yak, dromedary, llama, goats, sheep, elk, deer, moose, reindeer, cats, dogs, donkey, horse, rabbit, kangaroo, guinea pig, pigs and boars. In specific embodiments, the myogenic precursor cells are from farmed animals selected from pigs, cows, sheep, fish, chicken, ducks and shellfish. As used herein, the term “animal cells” refers to “non-human cells”.

In some embodiments, the myogenic precursor cells are obtained by biopsy of muscle, for example, the gastrocnemius muscle of a mammal or the pectoralis muscle of an avian species. Biopsied tissue can then be dissociated into cells by enzymatic and/or mechanical means.

Enzymatic dissociation can be effected by protein digestion (e.g. trypsin, pronase digestion), alone or in combination with collagenase and/or DNase treatment (for combination protocols, see, for example, Miersch et al, In Vit. Cell and Dev Biol-Animal, 54: 406-412, 2018). In specific embodiments, the biopsied tissue is dissociated by incubation with trypsin (e.g. Trypsin B), 0.25%. The trypsinized tissue can then be further dissociated by mechanical means. In specific embodiments, the enzymatically dissociated tissue is subjected to mechanical dissociation with a blunt instrument, such as a serological pipette. Individual cells can be obtained by straining the supernatants, gentle centrifugation to pellet the dissociated cells and resuspension of the pellets in proliferation (e.g. growth) medium. In some embodiments, dissociated muscle tissue is “pre-plated” on uncoated plates in order to reduce the number of fibroblasts.

Prior to induction of formation of multinucleate myotubes, the myogenic precursor cells (whether dissociated myogenic precursor cells from biopsy, or other, for example, embryonic stem cells or iPSCs/iMSCs) are typically cultured in proliferation medium, without inducing differentiation, to greatly increase the number of cells available for methods of the invention. Culturing of the myogenic precursors may include utilizing gases to optimize growth conditions independently in each culturing vessel or throughout the entirety of the system. Suitable gases include but are not limited to oxygen, carbon dioxide and the like. In addition, salts are used to optimize growth conditions for cells. Suitable salts include but are not limited to those of sodium, potassium, calcium and the like. The amount of salt used is consistent with ranges known in the art of tissue or cell culture. Cells need nutrients to grow; nutrients provide a source of carbon. Suitable carbon sources include but are not limited to glucose, glycerol, galactose, hexose, fructose, pyruvate, glutamine and the like. The amount of carbon source used is consistent with ranges known in the art of tissue or cell culture. The basal medium may also include buffer such as phosphate-buffered saline (PBS), tris(hydroxymethyl)aminomethane (TRIS), phosphate-citrate buffer, sorensen's phosphate butler, sodium citrate buffer, 4-(2-hydroxyethyl)-1-piperazineethanesulfonic acid (HEPES) and the like. Alternatively, carbon dioxide can be fed into the medium to control the pH. The pH is maintained at about 5.5 to about 7.5. Vitamins are used to optimize growth conditions for cells. Suitable vitamins include but are not limited to folic acid, nicotinamide, riboflavin, B12 and the like. The number and concentration of vitamins used is consistent with ranges known in the art of tissue or cell culture. Therefore, as stated above, the localized culture conditions can be independently controlled to optimize the growth of the cells within the respective culturing vessels.

Culture conditions can be further controlled by temperature. Even though mammalian cells are typically cultured at body temperature, that is at 37 degrees C., sometimes deviation from this temperature might be desirable, depending on cell type. Thus, the culturing vessels may be individually temperature controlled in the range of 20-38 degrees C. (e.g. from room temperature to near body temperature). Culture temperature may also be adjusted according to the source of the myogenic precursor cells (mammalian, reptilian, avian, etc.). Further control and optimization of culturing can be achieved by the adjustment of the perfusion, its speed, pressure and, in case of pulsatile flow, its pulse frequency and strength.

In some embodiments, the proliferation medium contains basal medium, with or without additional growth/development factors. In specific embodiments, the proliferation medium is a basal medium supplemented with antibiotics (e.g. Gentamycin), mammalian serum (e.g. Bovine or Fetal Bovine Serum) and L-Glutamine, for example, BioAmf-2 medium (Biological Industries, Israel). In some embodiments, the proliferation medium is a basal medium supplemented with growth factors which allow continued growth of the cells in culture without transition to differentiation (e.g. “differentiationless proliferation” through at least 3-5 passages). Growth factors useful for proliferation medium include, but are not limited to FGF2, IL-6, IGF1, VEGF, HGF, PDGF-BB, Somatotropin, TGF-beta1, Nodal collagenase, MMP1 and Forskolin. Thus, in some embodiments, the proliferation medium comprises one or more of FGF2, IL-6, IGF1, VEGF, HGF, PDGF-BB, Somatotropin, TGF-beta 1, Nodal collagenase, MMP1 and Forskolin

According to a preferred embodiment, the medium comprises serum or serum-replacement or other defined factors which can be used to facilitate cell proliferation.

As used herein the phrase “serum replacement” refers to a defined formulation, which substitutes the function of serum by providing cells with components needed for growth and viability.

Various serum replacement formulations are known in the art and are commercially available.

For example, GIBCO™ Knockout™ Serum Replacement (Gibco-Invitrogen Corporation, Grand Island, NY USA, Catalogue No. 10828028) is a defined serum-free formulation optimized to grow cells in culture. It should be noted that the formulation of GIBCO™ Knockout™ Serum Replacement includes Albumax (Bovine serum albumin enriched with lipids) which is from an animal source (International Patent Publication No. WO 98/30679 to Price, P. J. et al). However, a recent publication by Crook et al., 2007 (Crook J M., et al., 2007, Cell Stem Cell, 1: 490-494) describes six clinical-grade hESC lines generated using FDA-approved clinical grade foreskin fibroblasts in cGMP-manufactured Knockout™ Serum Replacement (Invitrogen Corporation, USA, Catalogue No. 04-0095).

According to some embodiments of the invention, the concentration of GIBCO™ Knockout™ Serum Replacement in the culture medium is in the range of from about 3% [volume/volume (v/v)] to about 50% (v/v), e.g., from about 5% (v/v) to about 40% (v/v), e.g., from about 5% (v/v) to about 30% (v/v), e.g., from about 10% (v/v) to about 30% (v/v), e.g., from about 10% (v/v) to about 25% (v/v), e.g., from about 10% (v/v) to about 20% (v/v), e.g., about 10% (v/v), e.g., about 15% (v/v), e.g., about 20% (v/v), e.g., about 30% (v/v).

Another commercially available serum replacement is the B27 supplement without vitamin A which is available from Gibco-Invitrogen, Corporation, Grand Island, NY USA, Catalogue No. 12587-010. The B27 supplement is a serum-free formulation which includes d-biotin, fatty acid free fraction V bovine serum albumin (BSA), catalase, L-carnitine HCl, corticosterone, ethanolamine HCl, D-galactose (Anhyd.), glutathione (reduced), recombinant human insulin, linoleic acid, linolenic acid, progesterone, putrescine-2-HCl, sodium selenite, superoxide dismutase, T-3/albumin complex, DL alpha-tocopherol and DL alpha tocopherol acetate.

Thus, in some embodiments, the myogenic precursor cells are undifferentiated myogenic precursor cells cultured in proliferation medium prior to inducing multinucleated myotube formation. In specific embodiments, the proliferation medium lacks factors active in inducing formation of the multinucleated myotubes. In some embodiments, the proliferation medium lacks one or more of EGF1, p38 agonists and TGFB inhibitors.

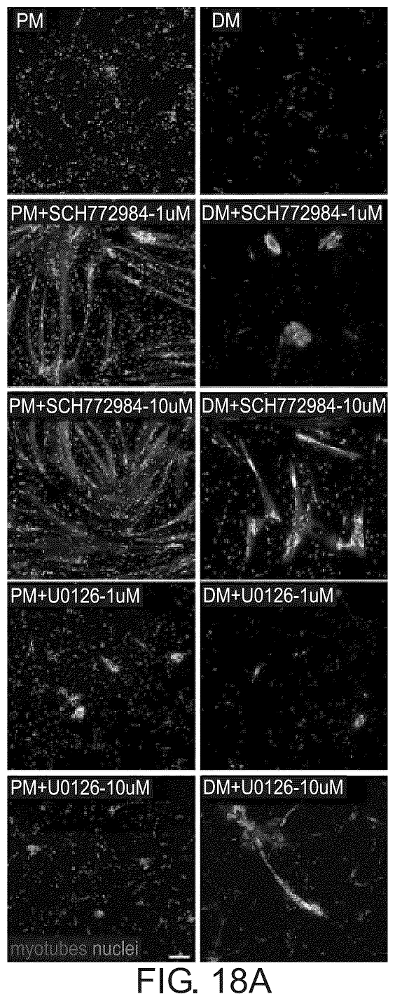

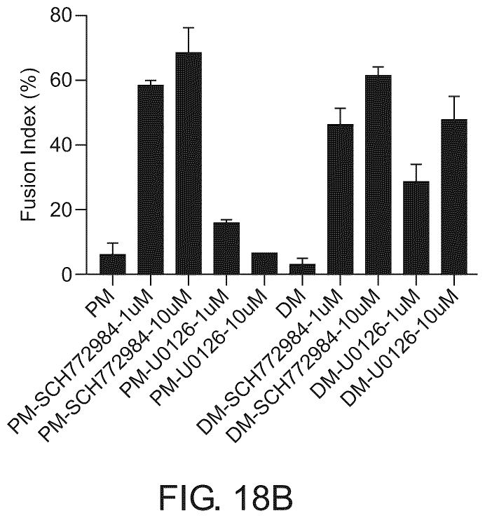

The present inventors have shown that addition of one or more of at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoid Acid Receptor (RAR) agonist, a Retinoid Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist, a Calcium ionophore and a Calmodulin-dependent Protein Kinase II (CaMKII) activator to myogenic precursors in differentiation medium can also greatly enhance the transition to multinucleated myotubes (see, for example, FIGS. 18A and 18B). Thus, in some embodiments, the method comprises contacting myogenic precursor cells which have been, or are being cultured in differentiation medium (which may or may not have been cultured previously in proliferation medium) with one or more of at least one molecule of the invention, thereby inducing or enhancing multinucleated myotube formation.

Expansion of the myogenic precursor cells can be performed in culture plates (e.g. petri dishes, “2D culture”), in culture vessels, in bioreactors and the like. In some embodiments, the myogenic precursor cells are expanded on coated plates or vessels, for example, coated with a reconstituted basement membrane (e.g. Matrigel). In other embodiments, the myogenic precursor cells are expanded on a substrate or scaffold, for “3D culture”.

In some cases, cells are cultured in suspension in cell culture flasks. The cell culture flasks are optionally stacked and/or arranged side-by-side as with the 2D surface cell culture. Cells cultured in suspension are usually non-adherent cells. In some cases, however, adherent cells are cultured on scaffolds in a suspension. Scaffolds provide structural support and a physical environment for cells to attach, grow, and migrate. In addition, scaffolds usually confer mechanical properties such as elasticity and tensile strength. Oftentimes, 3D scaffolds are used to culture adherent cells so as to enable 3D growth of the cells. Scaffolds sometimes have specific shapes or sizes for guiding the growth of the cultured cells. In some cases, scaffolds are composed of one or more different materials. Some scaffolds are solid scaffolds, while others are porous. Porous scaffolds allow cell migration or infiltration into the pores. Scaffolds are typically composed of a biocompatible material to induce the proper recognition from cells. In addition, the scaffold is made of a material with suitable mechanical properties and degradation kinetics for the desired tissue type that is generated from the cells. In some cases, a scaffold comprises a hydrogel, a biomaterial such as extracellular matrix molecule (ECM) or chitosan, or biocompatible synthetic material (e.g. polyethylene terephthalate). ECM molecules are typically proteoglycans, non-proteoglycan polysaccharides, or proteins. Potential ECM molecules for use in scaffolding include collagen, elastin, heparan sulfate, chondroitin sulfate, keratan sulfate, hyaluronic acid, laminin, and fibronectin. Sometimes, plant-based scaffolds are used for 3D culturing.

Normal cells in culture tend to proliferate until confluence, at which point contact inhibition blocks further divisions. Thus, in some embodiments, cells are cultured in proliferation medium until confluence. In other embodiments, expansion is prolonged by partial depletion of the cells, transfer to more spacious culture vessels or by prevention of confluence, e.g. spinner flasks, bioreactors.

In some embodiments expansion is performed for about 0.5, 1.0, 1.5, 2.0, 2.5, 3.0, 3.5, 4.0, 4.5, 5.0, 5.5, 6.0, 6.5, 7.0 days, 1.5 weeks, 2.0 weeks or more. In specific embodiments, the myogenic precursor cells are expanded in proliferation medium for 24 hours.

The mode of operation, be it batch, fed-batch or continuous will impact the bioreactor size and media requirements. From a large-scale perspective, typically fed-batch or continuous supply of media is favored. Several configurations can operate in these modes. Agitated vessels are currently the most common in biotech industries. They provide a time-averaged homogenous, well-mixed environment through convective mixing initiated by mechanical, pneumatic or hydraulic agitation such as impeller driven stirred tank bioreactors (STRs), rotating wall bioreactors (RWBs), and rocking motions as seen with wave bioreactors. Other bioreactor configurations enable continuous, perfusion operation such as packed bed bioreactors (PBBs), fluidized bed bioreactors (FBBs) and membrane bioreactors such as hollow fiber bioreactors (HFBs). For non-perfusion reactors, such as STRs, continuous (perfusion) operation requires the coupling of the bioreactor with an internal or external cell retention device on a recycle line, by centrifugation, sedimentation, ultrasonic separation or microfiltration with spin-filters, alternating tangential flow (ATF) filtration or tangential flow filtration (TFF). See also:
Clincke, M. F., Mölleryd, C., Samani, P. K., Lindskog, E., Fäldt, E., Walsh, K., et al. (2013a). Very high density of Chinese hamster ovary cells in perfusion by alternating tangential flow or tangential flow filtration in WAVE bioreactor™-part II: Applications for antibody production and cryopreservation. Biotechnol. Prog. 29, 768-777. doi: 10.1002/btpr.1703;
Clincke, M. F., Mölleryd, C., Zhang, Y., Lindskog, E., Walsh, K., and Chotteau, V. (2013b). Very high density of CHO cells in perfusion by ATF or TFF in WAVE bioreactor™: Part I: Effect of the cell density on the process. Biotechnol. Prog. 29, 754-767. doi: 10.1002/btpr.1704.

Bioreactors that are typically used for the expansion of muscle cells are described in Allen et al. ront. Sustain. Food Syst., 12 Jun. 2019 www(dot)doi(dot)org/10(dot)3389/fsufs(dot)2019(dot)00044.

Decisions related to the type, size and number of bioreactors are influenced by a number of factors including passaging, which are within the skills of the ordinary artisan and are described in a non-limiting manner by Allen et al. ront. Sustain. Food Syst., 12 Jun. 2019 |www(dot)doi(dot)org/10(dot)3389/fsufs(dot)2019(dot)00044. Passaging, in the form of sequential transference to reactors of increasing size, as seen in seed trains, is required to satisfy the minimum and maximum cell densities. Microcarrier culture and bead-to-bead transfer capability of a cell-line (Verbruggen et al., 2017 ytotechnology, 1-10. doi: 10.1007/s10616-017-0101-8) may enable passaging through the addition of microcarriers to increase surface area without increasing vessel size. Bioreactor comparisons are typically made based on final cell density achievable and not on the volume, an arbitrary concept without context such as the seeding density and final cell number or density and passaging steps. The achievable cell density will differ for suspension systems that use microcarriers for anchorage-dependent cells vs. single-cell suspension.

Following expansion of the myogenic precursor cells, the cells are washed and the proliferation medium is replaced with a medium having reduced amounts of proliferation-inducing factors compared to their concentrations in the proliferation media, and comprising factors supporting myogenesis, or, in other embodiments, the medium is supplemented with molecules for inducing formation of multinucleated myotubes from the myogenic precursors.

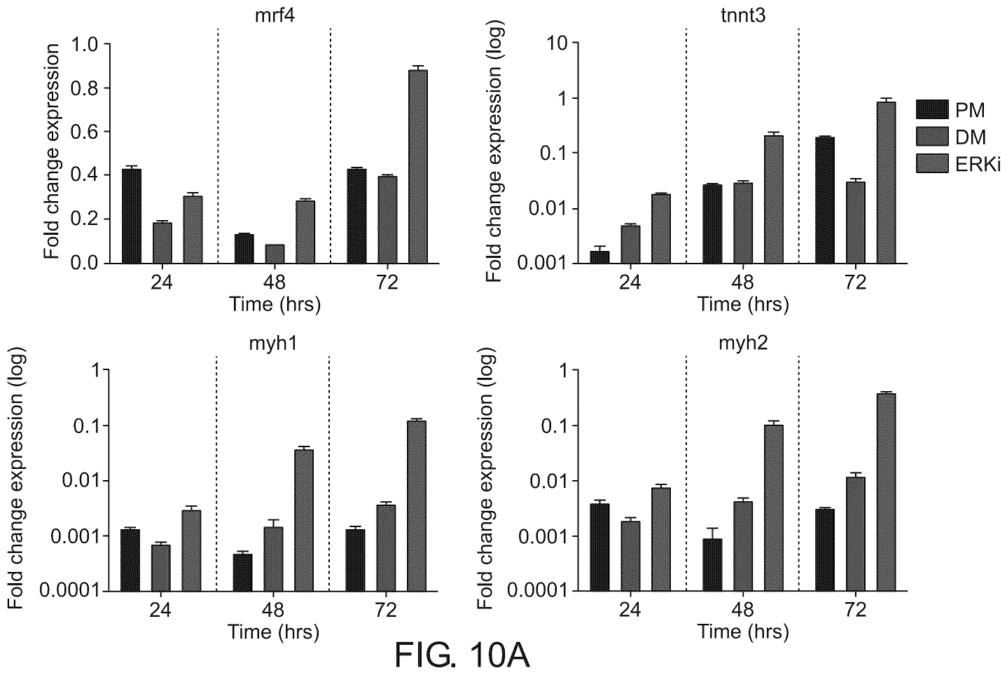

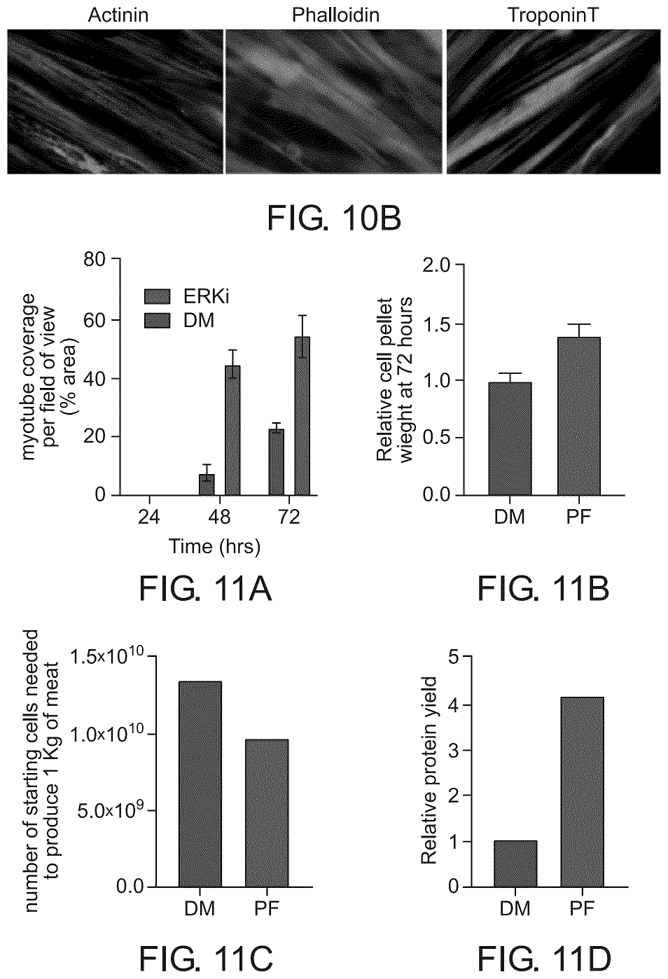

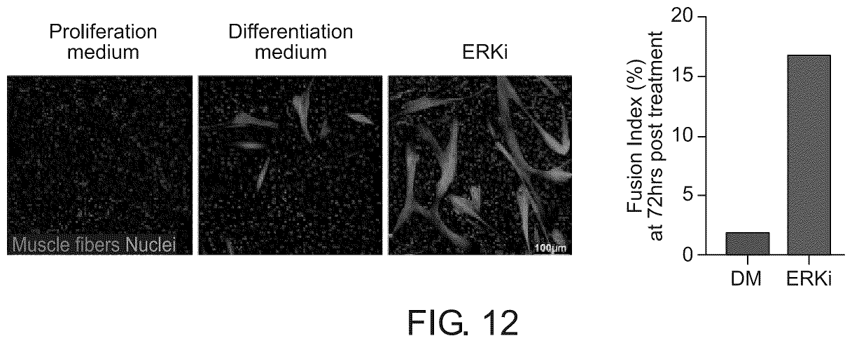

The present inventors have shown that ERK1/2 is a critical factor in maintaining the myogenic precursor character of the precursor cells, and that inhibition of ERK1/2 can induce formation of multinucleated myotubes from cultured myogenic precursors (see, for example, FIGS. 1A and 1E, and, in particular, FIG. 10B). Further, the present inventors have shown that additional factors constitute regulatory influences in the transition of myogenic precursors to fused, multinucleated myotubes. Typically, additional factors which can be added to induce transition of the myogenic precursors to fusion into fused, multinucleated myotubes include inhibitors of regulatory functions upstream of ERK1/2, and activators/agonists of regulatory functions downstream of ERK1/2.

Thus, in some embodiments, there is provided a method of inducing multinucleated myotube formation, the method comprising contacting myogenic precursor cells from a farmed animal with at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator.

In other embodiments, there is provided a method of inducing multinucleated myotube formation, the method comprising contacting myogenic precursor cells from a farmed animal with at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator wherein when the myogenic precursor cells are of chicken the contacting is performed in the presence of Extracellular Regulated Signaling Kinase (ERK1/2) inhibitor and an upregulator of intracellular Ca 2+.

Myogensis is the entire process which includes differentiation and fusion, and muscle fiber maturation. Differentiation is a distinct phase of the process. Undifferentiated and quiescent muscle progenitor/satellite cell express markers such as Pax3/Pax7. Progenitors become activated and begin to proliferate, signaling early differentiation into myoblasts. Myoblasts may express remnants of Pax3/Pax7, but characteristically begin to express the markers MyoD and Myf5. Late differentiation, when a myoblast exits the cell cycle, is marked by expression of markers such as MEF2 proteins and Myogenin (MyoG), and MRF4. The positive expression of these specific proteins/RNAs indicates their activation and stage of commitment. Classically, the marker used to evaluate commitment and therefore differentiation, is MyoG. Once differentiated into MyoG positive myoblasts, the cells also begin to express myosin heavy chain (MyHC) prior to undergoing fusion with other myoblasts and at this stage are considered “differentiated” and “fusion competent”. However, differentiated cells may also remain unfused as mononucleated myocytes (myosin heavy chain positive and MyoG positive). One will appreciate that cell cultures are a population of cells, and that maturation is a dynamic process—thus, although expression of markers in a culture is subject to a statistical distribution, and you may find cells in cultures that are in transitionary phases that express markers of different stages simultaneously, culture conditions such as those of the invention can reproducibly provide cell populations rich in multinucleated myotubes expressing characteristically high levels of maturation markers compared to those found in only differentiated cells.

In some embodiments, the at least one molecule is an ERK1/2 inhibitor. Inhibitors of ERK1/2 suitable for use with the methods of the present invention include, but are not limited to MK-8353 (SCH900353), SCH772984, CC-90003, Corynoxeine, ERK1/2 inhibitor 1, magnolin, ERK IN-1, ERK IN-2, ERK IN-3, LY3214996, Ravoxertinib, Ravoxertinib hydrochloride, VX-11e, FR 180204, Ulixertinib, Ulixertinib hydrochloride, ADZ0364, K0947, FRI-20 (ON-01060), Bromacetoxycalcidiol (B3CD), BVD523, DEL22379, FR180204, GDC0994, K0947, AEZ-131(AEZS-131), AEZS-136, AZ-13767370, BL-EI-001, LTT, ASTX-029, TCS ERK 11e and CAY10561. In some embodiments the ERK inhibitors are selected from the peptide inhibitors EPE, ERK Activation Inhibitor Peptide I (ERK inhibitor IV) and ERK Activation Inhibitor Peptide II (ERK inhibitor V). In specific embodiments, the ERK1/2 inhibitor is SCH772984.

In some embodiments, the at least one molecule is an inhibitor of ERK1/2 upstream regulators, including but not limited to MEK1 inhibitors. Inhibitors of MEK1 suitable for use with the methods of the present invention include, but are not limited to Trametinib, PD98059, U0126 (U0126-EtOH), PD0325901, Selumetinib (AZD6244), Cobimetinib (GDC-0973, RG7420), Binimetinib (MEK162), CI-1040 (PD 184352), Refametinib (BAY 869766; RDEA119), Pimasertib (AS703026), Selumetinib (AZD6244), Cobimetinib hemifumarate, GDC-0623 (RG 7421), R04987655, AZD8330(ARRY-424704), SL327, MEK inhibitor, PD318088, Cobimetinib racemate (GDC-0973 racemate; XL518 racemate), PD198306, AS-703026, ADZ8330 and EBI-1051.

In some embodiments, the at least one molecule is an inhibitor of mitogens such as FGF1 and its receptors, including but not limited to FGF inhibitors. Inhibitors of FGF suitable for use with the methods of the present invention include, but are not limited to Derazantinib, PD 161570, SSR 128129E, CH5183284, PD 166866 and Pemigatinib.

In some embodiments, the at least one molecule is an inhibitor of TGF beta and its receptors, including but not limited to TGF beta inhibitors. Inhibitors of TGF beta suitable for use with the methods of the present invention include, but are not limited to SD208, LY364947, RepSox, SB 525334, R 268712 and GW 788388.

In some embodiments, the at least one molecule is a molecule which activates or acts as an agonist to Retinoid-X Receptors (RXR) and/or Retinoic Acid Receptors (RAR), including but not limited to RXR/RAR agonists. Agonists of RXR/RAR suitable for use with the methods of the present invention include, but are not limited to CD3254, Docosahexaenoic acid, LG100268, SR11237, AC261066, AC55649, Adapalene, BMS961, CD1530, CD2314, CD437, BMS453, EC23, all-trans retinoic acid, all-trans-4-hydroxy retinoic acid, all-trans retinoic acid-d5, cyantraniliprole, Vitamin A, all-trans retinol, LG100754, Beta Carotene, beta-apo-13 carotene, lycopene, all-trans-5,6-epoxy retinoic acid, all-transe-13,14-Dihydroretinol, Retinyl Acetate, Hanokiol, Valerenic acid, HX630, HX600, LG101506, 9cUAB30, AGN194204, LG101305, UVI3003, Net-41B, CBt-PMN, XCT0135908, PA024, methoprene acid, 9-cis retinoic acid, AM80, AM580, and CH55, TTNPB, and Fenretinide, LG-100064, Fluorobexarotene (compound 20), Bexarotene (LGD1069), Bexarotene D4, NBD-125 (B-12), LGD1069 D4 and 9-cis-Retinoic acid (ALRT1057).

In some embodiments, the at least one molecule is a molecule which increases expression of or acts as an agonist to Ryanodine Receptors (RYR1 and RYR3), including but not limited to RYR agonists. Agonists of RYR suitable for use with the methods of the present invention include, but are not limited to Caffeine, Chlorocresol, CHEBI:67113, chlorantraniliprole, S107hydrochloride, JTV519, Trifluoperazine (T FP), Xanthines, Suramin, Suramin sodium, NAADP tetrasodium salt, S100A1, Cyclic ADP-Ribose (ammonium salt), pentifylline, 4-chloro-3-methylphenol (4-chloro-m-cresol), tetraniliprole, trifluoperazine (TFP), cyclaniliprole and Cyantraniliprole. In specific embodiments, the RYR agonist is a methyl xanthine. In particular embodiments, the RYR agonist is caffeine.

In some embodiments, the at least one molecule is a molecule which activates or acts as an agonist of or upregulates cytoplasmic levels of Ca 2+, including but not limited to upregulators of intracellular calcium 2+. Upregulators of Ca 2+ suitable for use with the methods of the present invention include, but are not limited to NAADP tetrasodium salt, Cyclic ADP-Ribose, 4-bromo A23187, lonomycin, A23187 and isoproterenol.

In some embodiments, the at least one molecule is a molecule which activates or acts as an agonist of CaMKII, including but not limited to agonists of CaMKII. Agonists of CaMKII suitable for use with the methods of the present invention include, but are not limited to Calcium, Calmodulin, CALP1 and CALP3. In other embodiments, the at least one molecule is a molecule which regulates downstream targets of CaMKII. Such molecules which regulate downstream targets of CaMKII and are suitable for use with the present invention include but are not limited to IRSP53, RAC1, CDC42, SRF, CREB, Actin, Kalirin-7, SynGap, Myomaker and Tiaml.

In still other embodiments, the at least one molecule is a molecule which upregulates cytoplasmic levels of Ca 2+ is a calcium ionophore. Calcium ionophores suitable for use with the present invention include but are not limited to ionomycin, calcimycin, calcium ionophore I (CA1001; ETH1002), B eauvericin, Laidlomycon, Lasalocid, Salinomycin and Semduramycin.

Sarcoendoplasmic calcium-ATPase is an intracellular membrane transporter that actively transports Ca 2+ ions from the cytosol to the lumen of the sarco(endo)plasmic reticulum. Inhibiting the SERCA channel activity may enhance cytosolic calcium retention. Thus, in still other embodiments, the at least one molecule is a sarcoendoplasmic calcium-ATPase (SERCA) inhibitor. SERCA inhibitors suitable for use with the present invention include, but are not limited to cyclopiazonic acid, 2,5-Di-tert-butylhydroquinone, (DBHQ), Ruthenium red, t-Butylhyroquinone, Gingerol, CPG 37157, Thapsigargin and Paxilline.

The molecules may be contacted with the myogenic precursors individually, or in combination with other suitable molecules. In specific embodiments, the myogenic precursors are contacted with ERK1/2 inhibitors, or upregulators of intracellular Ca 2+, or both ERK1/2 inhibitors and upregulators of intracellular Ca 2+. In specific embodiments, when the myogenic precursors are from (e.g. derived from) chicken, the contacting is performed in the presence of ERK1/2 inhibitor and an upregulator of intracellular Ca 2+.

Culture of the myogenic precursors for induction of formation of the multinucleated myotubes can be carried out in vessels or plates or bioreactors as described for expansion of the myogenic precursor cells with proliferation medium. Briefly, induction of multinucleated myotube formation can be performed in culture plates (e.g. petri dishes, “2D culture”), in culture vessels, in bioreactors and the like. In some embodiments, the myogenic precursor cells are expanded on coated plates or vessels, for example, coated with a reconstituted basement membrane (e.g. Matrigel). In other embodiments, the myogenic precursor cells are expanded on a substrate or scaffold, for “3D culture”.

It is important to note that, since the cultured multinucleated myotubes can be incorporated in a cultured meat or cultured muscle composition, the use of 3D scaffolds can be effective. Scaffolds sometimes have specific shapes or sizes for guiding the growth of the cultured cells. The scaffold is made of a material with suitable mechanical properties and degradation kinetics for the desired tissue type that is generated from the cells. In certain instances, a scaffold comprises a degradable material to enable remodeling and/or elimination of the scaffold in the cultured food product. For example, in some cases, a 3D scaffold that shapes cultured myotubes into the shape of a meat patty biodegrades after the myotubes expand to fill up the interior space of the scaffold. In other instances, the scaffold comprises a material that remains in the cultured food product. For example, sometimes, at least a portion of a collagen scaffold providing support to cultured myocytes remains to provide texture and continuing structural support in the cultured food product. In some cases, a scaffold comprises a hydrogel, a biomaterial such as extracellular matrix molecule (ECM) or chitosan, or biocompatible synthetic material (e.g. polyethylene terephthalate).

Multinucleated myotube formation can be accompanied by increased expression or activity of differentiation-related factors. Skeletal muscle markers include, but are not limited to alpha-, beta- and epsilon-Sarcoglycan, Calpain inhibitors, Creatine kinase MM/CKMM, elF5A, Enolase2/Neuron-specific Enolase, FABP3/H-FABP, GDF-8/Myoststin, GDF-11/GDF8, MCAM/CD146, MyoD, Myogenin, Myosin light chain Kinase Inhibitors, Troponin 1, Troponin1/Tnn13. Thus, in some embodiments, culturing of the myogenic precursors in medium comprising at least one of Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist, SERCA inhibitor and a Calmodulin-dependent Protein Kinase II (CaMKII) activator results in increased expression of myogenic differentiation factors including, but not limited to MyoD, MyoG, Mymk, Mymx, troponin (Tnnt), Myosin heavy chain 1 and 2 (MyHC 1, MyHC 2) and Actinin. In some embodiments, inducing multinucleated myotubes results in an increased fraction of MYOG-positive nuclei in the cultured myogenic precursors, as compared to nuclei of myogenic precursors cultured in serum-depleted differentiation medium lacking or devoid of the at least one of Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator.

Thus, according to some aspects of the invention, there is provided a cultured meat composition originating from myogenic precursor or progenitor cells, characterized by enhanced myogenic markers, compared with identical cells cultured for the same duration without the at least one of Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator of the invention.

In some embodiments, the cultured meat composition is characterized by abundant mature muscle fibers and presence of characteristic striation of the actin, troponin and phalloidin signals, already after as few as 24 hours in culture according to the methods of the invention. Such striation is indicative of organization of the multinucleated myotubes into sarcomeric architecture. In other embodiments, the cultured meat composition is characterized by increased expression and activation of CaMKII and Ryodine receptors (RYR), compared with identical cells cultured, for the same duration, without the at least one of Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator of the invention.

In still other embodiments, the cultured meat composition is characterized by increased expression of myogenic markers including, but not limited to myosin heavy chain (MyHC), MyoG, desmin, dystrophin and laminin, compared with identical cells cultured for the same duration without the at least one of Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator of the invention.

In still other embodiments, the cultured meat composition is characterized by increased presence of multinucleated myotubes (indicating a greater fusion index), compared with identical cells cultured for the same duration without the at least one of Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator of the invention.

In some embodiments, the duration of culture (e.g. before comparison of muscle maturation characteristics) is 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70 or 72 hours or more, one, two, three, four, five, six, seven, eight, ten, twelve, fourteen or more days. In some embodiments, cultures can be compared for myogenic characteristics after 1, 2, 4, 6, 8, 12, 16, 20, 24, 36, 48 or 72 hours.

The present inventors have uncovered that culturing the myogenic precursors with the indicated molecules results in rapid and robust fusion, efficiently producing multinucleated myotubes in a surprisingly short time (see, for example, FIGS. 4A-4E), in a matter of hours, rather than days. Thus, in some embodiments, the contacting of the myogenic precursors with the at least one molecule of the invention is effected for 10-96 hours, 12-72 hours, 12-48 hours, 18-48 hours, 18-36 hours, 24-28 hours, 16-48 hours or 16-24 hours. In specific embodiments, contacting the myogenic precursors with the at least one molecule of the invention is effected for 12-48 hours, or 16-24 hours.

It will be noted that the methods of the invention result in synchronization of the transition from mono- and bi-nucleated myogenic precursors to multinucleated myotubes, as well as significantly increasing the efficiency of formation of multi-nucleated myotubes from the myogenic precursors. Thus, in some embodiments, contacting the myogenic precursors with the at least one molecule of the invention is effected until 30%, 40%, 50%, 60% or more of the nuclei in the culture are from multinucleated myogenic precursors. In other embodiments, contacting the myogenic precursors with the at least one molecule of the invention is effected until at least 50% of the nuclei in the culture are from multinucleated myogenic precursors.

It will be appreciated that the myogenic precursors or progenitors cultured according to the methods disclosed herein can have characteristic and/or unique gene expression patterns or temporal patterns, which may be distinct from those of myogenic precursors or progenitors not cultured according to the methods of the invention.

Thus, in some embodiments, the cultured myogenic precursor or progenitor cells cultured according to the methods of the invention are characterized by at least one of a gene expression profile, an RNA profile (e.g. transcriptosome) and/or a protein profile (e.g. proteasome) distinct from that of those of myogenic precursors or progenitors not cultured according to the methods of the invention. Such profiles can be produced using commercially available (e.g. Affymetrix Gene Chips®) or custom arrays.

The methods of the present invention can be used to produce multinucleated myotubes suitable for use as cultured meat.

The present teachings are particularly valuable for the meat industry where large amount of cells are required at minimal commodity costs.

As mentioned hereinabove, and further described in the Examples section which follows, the present inventors were able to demonstrate enhanced yield in terms of fiber yield, protein yield and cell-weight yield (see FIGS. 11A-D). Ultimately this implies that less growth medium (and resources in general) would be required to produce the same amount of product in the same amount of time; therefore, reducing overall costs of the production process.

According to some embodiments of the invention, an exemplary process for obtaining myotubes is described in the Examples section which follows. Briefly, a muscle biopsy is obtained. A primary culture is subjected to 1 or more (e.g., 2-3) steps of preplating to remove fibroblasts and enrich for myoblasts in the presence of a proliferation medium (a medium which allows proliferation as known to those of skills in the art). Then molecule(s) as described herein, e.g., ERKi, RXR/RAR agonist, Ryanodine receptor agonist (RYR), CaMKII inhibitors, calcium ionophores or combinations thereof is added to the culture for a predetermined period of time after which the cells are washes and cultured again in the presence of a proliferation medium in the absence of the molecules.

It will be appreciated that the molecules as described herein (“at least one of . . . ”) can also be used for obtaining multinucleated myotubes from myogenic precursors or progenitors while being cultured in differentiation medium, in order to enhance (e.g. increase or quicken) fusion and development of the multinucleated myotubes.

When sufficient amount of cells is obtained, cultures of multinucleated myogenic precursors can be harvested to provide biomass for cultured meat compositions. In some embodiments the cultures are harvested before “maturity” (fewer than 100% of the cells are multinucleated), and in other embodiments, the cultures are harvested at “maturity”, i.e. substantially all of the cells are multinucleated.

At any stage (i.e., myoblast to myotube) the cells can be harvested and banked for further use.

Thus, according to some embodiments there is provided a cultured meat composition comprising the multinucleated myotubes produced by the methods of the invention described herein.

In producing the cultured meat composition, the desired biomass of multinucleated myotubes may be a biomass reached once the cells are no longer able to proliferate or may be the maximum biomass the cells can reach in a given culture size and culture conditions. In some embodiments, the biomass of multinucleated myotubes is that at which at least 50%, 60% or more of the nuclei in the culture are from multinucleated myogenic precursors. Alternatively, the desired biomass may be the biomass at which sufficient cells have been produced to form a cultured meat composition.

Cultured meat compositions, cultured meat products, manufactured meat compositions or products, and cultivated meat compositions or products refer to meat compositions or products that contain animal cells grown outside the animal in plates, vessels, flasks, bioreactor systems or other similar production systems. Cultured meat compositions or products can take numerous forms and be used in different ways. Manufactured or cultured animal cells can be used as ingredients to foods containing a high percentage of vegetable material, or they can be produced in enough biomass to be the primary ingredient in the food. Cultured meat compositions or products may also contain other ingredients or additives, including but not limited to preservatives.

Thus, in some embodiments, there is provided a comestible comprising the cultured meat composition of the invention. As used herein, the term “comestible” refers to an item of food, an edible item. In specific embodiments, the cultured meat composition or comestible comprising the cultured meat composition is suitable for human or animal consumption.

The cultured meat compositions or comestibles of the invention may comprise tissue engineered products, cultured animal cells blended with plant-based protein, or pure animal cell products. In some embodiments, cultured meat compositions or comestibles include cultured animal cells that may or may not be combined with plant-based protein or other food additives or ingredients, may result in unstructured ground meat products, such as ground beef, or may be tissue engineered/synthesized into structured tissue such as bacon or steak. In some embodiments, in addition to the multinucleated myotubes, the cultured meat or comestibles can comprise additional cells including, but not limited to adipocytes, muscle cells, blood cells, cartilage cells, bone cells, connective tissue cells, fibroblasts and/or cardiomyocytes, and/or additional plant- or animal originated foodstuffs. Cultivated meat compositions can be structured into living tissue that can be matured in a bioreactor, or nonliving tissue as the end product.

In some cases, comestibles of the invention may be combined with or substantially composed of vegetable matter. Sources of vegetable matter which may be used include, without limitation, peas, chickpeas, mung beans, kidney beans, fava beans, soy, cowpeas, pine nuts, rice, corn, potato, and sesame. Exemplary methods for producing hybrid comestible compositions comprising cultured meat compositions and plant-based or plant-derived components (e.g. plant protein) are detailed in US Patent Application Publication 20200100525.

A comestible comprising the cultured meat composition of the invention may have an increased meat-like flavor, aroma, or color, compared to a cultured meat product comprising a same number of unmodified cells of the same type. A comestible of the invention comprising both a plant-based product and the cultured meat composition of the present invention may have an increased meat-like flavor, aroma, or color, compared to a plant based product without the cultured meat composition of the invention.

The cultured meat composition and comestible can be enriched to some degree, when required, with additives to protect or modify its flavor or color, to improve its tenderness, juiciness or cohesiveness, or to aid with its preservation. Cultured meat additives hence potentially include, inter alia, salt and other means to impart flavor and inhibits microbial growth, extends the product's shelf life and helps emulsifying finely processed products, such as sausages. Nitrite is utilizable in curing meat to stabilize the meat's color and flavor, and inhibits the growth of spore-forming microorganisms such as C. botulinum. Phosphates used in meat processing are normally alkaline polyphosphates such as sodium tripolyphosphate. Erythorbate or its equivalent ascorbic acid (vitamin C) is utilizable to stabilize the color of cured meat. Sweeteners such as sugar or corn syrup impart a sweet flavor, bind water and assist surface browning during cooking in the Maillard reaction. Seasonings impart or modify flavor. They include spices or oleoresins extracted from them, herbs, vegetables and essential oils. Flavorings such as monosodium glutamate impart or strengthen a particular flavor. Tenderizers break down collagens to make the meat more palatable for consumption. They include proteolytic enzymes, acids, salt and phosphate. Dedicated antimicrobials include lactic, citric and acetic acid, sodium diacetate, acidified sodium chloride or calcium sulfate, cetylpyridinium chloride, activated lactoferrin, sodium or potassium lactate, or bacteriocins such as nisin. Antioxidants include a wide range of chemicals that limit lipid oxidation, which creates an undesirable “off flavor”, in precooked meat products. Acidifiers, most often lactic or citric acid, can impart a tangy or tart flavor note, extend shelf-life, tenderize fresh meat or help with protein denaturation and moisture release in dried meat. They substitute for the process of natural fermentation that acidifies some meat products such as hard salami or prosciutto.

It is thus within the scope of the invention wherein the comestible or cultured meat composition additionally comprises Acidity regulators, Alkalinity regulators, Anticaking agents, Anticaking agents, Antifoaming agents, Antifoaming agents, natural and other Antioxidants, Bulking agents, Food coloring agents, color retention agents, Emulsifiers, Flavors, Flavor enhancers, Flour treatment agents, Glazing agents, Humectants, Tracer gas, Preservatives, Probiotic microorganisms, Stabilizers, Sweeteners, Thickeners and any mixtures thereof. In particular embodiments, the additives are certified GRAS additives.

In another embodiment of the present invention, the comestible or cultured meat composition has the final organoleptic properties of a meat product, and especially product(s) selected from the group consisting of Beef, Beef heart, Beef liver, Beef tongue, Bone soup from allowable meats, Buffalo, Bison, Calf liver, Caribou, Goat, Ham, Horse, Kangaroo, Lamb, Marrow soup, Moose, Mutton, Opossum, Organ Meats, Pork, Bacon, Rabbit, Snake, Squirrel, Sweetbreads, Tripe, Turtle, Veal, Venison, Chicken, Chicken Liver, Cornish Game Hen, Duck, Duck Liver, Emu, Gizzards, Goose, Goose Liver, Grouse, Guinea Hen, Liver, Ostrich, Partridge, Pheasant, Quail, Squab, and Turkey.

According to an embodiment, the comestible or cultured meat composition has enhanced a meat organoleptic property or meat nutritional property, greater than cultured meat compositions devoid of the multinucleated myoblasts cultured according to the disclosed methods. As used herein, organoleptic properties refer to the aspects of food (or other substances) as experienced by the senses, including taste, sight, smell and touch. Exemplary organoleptic properties include, but are not limited to taste, odor, texture and color. Methods of organoleptic assaying are well known in the art, some of which are described infra.

Organoleptic (sensory) evaluation is a common and very useful tool in quality assessment of processed food (e.g., meat, cultured meat) products. It makes use of the senses to evaluate the general acceptability and quality attributes of the products. The assays typically make use of dedicated panelists and/or artificial means.

Common test methods used in sensory evaluation are: 1. Paired comparison test for simple difference where two coded samples are presented to the panelists for evaluation on simple difference. 2. Triangle test where three coded samples are presented at the same time, two are identical and the third is odd and the panelist is asked to identify the odd sample. 3. Hedonic scale rating test or acceptability test where samples are tested to determine their acceptability or preference.

Sensory testing (chewing) is normally sufficient to test tenderness/toughness or homogenous/fibrous structure of meat and meat products. If more objective results are desired, special instruments for texture measurement can be employed. Such a device typically measures the shear-force necessary to cut through meat/meat products. Comparative texture measurements are usually taken from same tissues or products which were submitted to different treatments such as ripening, cooking etc.

The list of relevant sensory attributes includes three main groups, adjusted individually per type of product, as follows: Appearance: surface color, internal color, texture (coarseness, uniformity), overall rating with relevance to the type of product tested. Texture: hardness/softness, juiciness/dryness, cohesiveness, chewiness, fatty/oily mouthfeel, overall rating. Taste and flavor (possible list of positive and negative characteristics of aroma and taste): meaty, cooked chicken, roasted chicken, bouillon-like (brothy), greasy, burned, sweet, bitter, rancid, overall rating.

The present invention further provides a method of producing a food or a food product, comprising steps of: a. providing a cultured meat composition or comestible as described herein and b. forming the cultured meat composition or comestible into a desired form. Further steps can include the addition of components for nutrition, flavor, taste, texture, color, odor, shelf life, etc.

According to some embodiments, the food or food product of the invention comprises cultured meat composition or comestible disclosed herein in the range from about 1% to about 99%, from about 2% to about 95%, from about 3% to about 92%, from 4% to about 90%, from about 5% to about 87%, from about 6% to about 85%, from about 7% to about 82%, from about 8% to about 80%, from about 9% to about 77%, from about 10% to about 75%, from about 12% to about 70%, from about 13% to about 65%, from about 15% to about 60%, from about 18% to about 55%, from about 20% to about 50%, from about 23% to about 45%, from about 25% to about 43%, from about 30% to about 40%. In other embodiments, the food or food product of the invention comprises cultured meat composition or comestible disclosed herein in the range from about 1% to about 10%, from about 10% to about 20%, from about 20% to about 30%, from 30% to about 40%, from about 40% to about 50%, from about 50% to about 60%, from about 60% to about 70%, from about 70% to about 80%, from about 80% to about 90%, from about 90% to about 99%, or 100%.

It is included in any of the methods, known in the art steps of cooking, sterilizing, pasteurizing, packaging and storing the food or food product.

Also provided is a method of providing nutrition to a subject in need thereof. The method comprising providing the subject with a food comprising cultured meat composition or comestible in an amount so as to enhance the nutrition of the subject. According to a specific embodiment, the subject is at risk of nutritional deficiency. According to a specific embodiment, the subject is a healthy subject (e.g., not suffering from a disease associated with nutrition/absorption).

According to a specific embodiment, the subject suffers from malnutrition. According to a specific embodiment, the subject suffers from a disease associated with nutrition/absorption e.g., hypocobalaminemia, iron deficiency anemia, zinc deficiency and vitamin D deficiency, fatty acid deficiency.

As mentioned, formation of multinucleated myotubes from myogenic precursors is a critical stage in regeneration of muscle tissue. As such, the methods disclosed herein for enhancing formation of the multinucleated myotubes can be used for treating muscle injury, where muscle regeneration is desirable.

Thus, according to some embodiments, there is provided a method of treating a muscle injury in a farmed animal, the method comprising contacting injured muscle tissue of the farmed animal with at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist and a Calmodulin-dependent Protein Kinase II (CaMKII) activator, thereby inducing myotube regeneration and treating the muscle injury, wherein when the myogenic precursor cells are of chicken the contacting is performed in the presence of Extracellular Regulated Signaling Kinase (ERK1/2) inhibitor and an upregulator of intracellular Ca 2+.

In specific embodiments, the method comprises contacting the injured muscle tissue with an ERK1/2 inhibitor and an upregulator of intracellular Ca 2+.

In some embodiments, the muscle injury can be, but is not limited to a bruise, a laceration, a contusion, pathological degenerative process, inflammation, ischemic injury, auto-immune injury or bacterial, parasitic or viral infection.

As bused herein, the term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes any farmed animals, at any age which suffer from the pathology. Preferably, this term encompasses individuals who are at risk to develop the pathology. The methods of treating as disclosed are exclusively for farmed animals, and treatment of humans is explicitly excluded.

As used herein the phrase “treatment regimen” refers to a treatment plan that specifies the type of treatment, dosage, schedule and/or duration of a treatment provided to a subject in need thereof (e.g., a subject diagnosed with a pathology). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., a damage to healthy cells or tissue). The type of treatment can include a surgical intervention (e.g., removal of lesion, diseased cells, tissue, or organ), a cell replacement therapy, an administration of a therapeutic drug (e.g., receptor agonists, antagonists, hormones, chemotherapy agents) in a local or a systemic mode, an exposure to radiation therapy using an external source (e.g., external beam) and/or an internal source (e.g., brachytherapy) and/or any combination thereof. The dosage, schedule and duration of treatment can vary, depending on the severity of pathology and the selected type of treatment, and those of skills in the art are capable of adjusting the type of treatment with the dosage, schedule and duration of treatment.

It is expected that during the life of a patent maturing from this application many relevant methods for culturing myogenic precursors will be developed and the scope of the term cultured meat is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

Mice. All experiments were approved by the Animal Care and Use Committee of the Weizmann Institute of Science (IACUC application #00720120-4). To generate satellite cell specific and tamoxifen inducible CaMKδ/γ double KO mice were, Pax7-Cre^{ERT} mice (Murphy et al., 2011) The Jackson laboratory, stock no. 017763) with double floxed CaMK2δ^{fl/fl}/γ^{fl/fl} mice (Kreusser et al., 2014). Pax7 Cre^{ERT/+}; CaMK2δ^{fl/fl}/γ^{fl/fl} (scDKO) or Pax7^{+/+}; CaMK2δ^{fl/fl}/γ^{fl/fl} (WT) littermates were used. Wildtype c57/b16 mice were purchased from ENVIGO. Nuclear and membrane reporter mice were bred inhouse by crossing nTnG^{+/+} and mTmG^{+/+} mice (The Jackson laboratory, stock no 023537, 007576 respectively). Actin/nuclear reporter mice were bred inhouse by crossing LifeAct-GFP mice (Riedl et al., 2008) with nTnG^{+/+} mice, calcium reporter mice were bred inhouse by crossing Pax7-Cre^{ERT+/+} (The Jackson laboratory, stock no. 017763) with GCaMP6^{flstop/flstop} (The Jackson laboratory, stock no. and 028866), tdTomato reporter mice were bred inhouse by crossing Pax7-Cre^{ERT+/+} with tdTomato^{flstop/flstop} Genotyping was performed on every litter.

Isolation of and treatment of primary myoblasts. Primary mouse myoblasts were isolated from gastrocnemius muscle using trypsin-tissue dissociation. Briefly, muscle tissues were incubated in Trypsin B (0.25%, Biological Industries) and subjected to mechanical dissociation with a serological pipet. Supernatants were strained and centrifuged. Pellets were grown resuspended in BioAmf-2 media (Biological Industries, Israel) and plated on 10% Matrigel (BD Biosciences)-coated plates at 37° and 5% CO₂. For all in vitro experiments, proliferation medium was Bio-Amf2 (Biological Industries, Israel) and Differentiation medium (DM) was DMEM 2% Horse Serum (HS) with 1% Pen/Strep. For fusion assays, cells were trypsinized with Trypsin C (0.05%, Biological Industries) and subjected to two rounds of preplating on uncoated plates to reduce the number of fibroblasts. Cells were plated at a density of 8×10³ per well in 10% Matrigel-coated 96-well plates in proliferation medium for 24 hours. The following day, proliferation media was replaced either with proliferation media or with DM containing DMSO (Ctrl) or 1 μM ERK1/2 inhibitor (ERKi; SCH772984, Cayman Chemicals), 2004 RXR antagonist (RXRi; HX-531, Cayman Chemicals), 5004 Ryanodine receptor antagonist (RYRi; Dantrolene, Cayman Chemicals), 5 μM CaMKII inhibitor (CaMKIIi; KN93, Cayman Chemicals), or with DM for controls.

Immunofluorescence staining. First passage primary myoblasts isolated from various strains (indicated in figure legends) were plated in 96-well plates or chamber slides and treated as previously described. The cells were fixed with ice cold 4% PFA in PBS for 10 minutes, permeabilized with 0.5% Triton X-100 in PBS for 6 minutes, and blocked in PBS with 0.025% tween, 10% normal horse serum and 10% normal goat serum for 1 hour at room temperature. Primary antibody incubation was done in blocking buffer overnight at 4 degrees, with the following antibodies: Myosin Heavy Chain (MyHC, MF20, DSHB hybridoma supernatant 1:10, or MY-32 ABCAM ab51263 1:400), Myogenin (MYOG sc-13137 SCBT 1:200), pHistone 3 (PH3, ab47297 ABCAM 1:1000), Ki-67 (Cell Marque #275R), RYR (ab2868 ABCAM 1:100), and pCaMKII (SIGMA SAB4504356 1:100). Cells were washed 3 times in PBS with 0.025% tween and then incubated with appropriate secondary antibodies in PBS 1 hour. Where indicated, nuclei were either labeled with DAPI (SIGMA D9542, 5 ug/ml) or Hoechst 33342 (Thermo scientific #62249, 1:2000). Fixed cells at 24 hours post treatment with indicated inhibitors were imaged using the Nikon Eclipse Ti2 microscope (further described in microscopy section). All analysis was performed on at least 1000 nuclei. For fixed cells following the timecourse with ERKi or DM (FIG. 1B), images were captured with an inverted Olympus IX83 microscope (further details in microscopy section). All imaging analysis were performed on at least 1000 cells.

Generation of retroviruses and transduction for live-cell imaging. 24 hrs prior to transfection, 3×10⁶ cells Platinum E Cells (Cell Biolabs) were seeded in 100-mm culture dish. 10 μg of retroviral plasmid DNA was transfected using FuGENE 6 (Roche). The viral suspension was collected from the conditioned media 48 hrs post transfection. The medium was centrifuged (2500 RPM/10 mins) to remove cell debris. The clarified viral suspension was used to transduce primary myoblasts. Briefly, 30,000 first passage primary myoblasts were seeded per well of a 6-well plate, 48 hrs prior to transduction using Polybrene (6 m/mL) (Merck: #TR 1003-G) as a transduction reagent. 1.5 hrs after infection, viral suspension was removed, cells were washed with PBS, and fresh Bioamf-2 culture media was added to cells. 24 hrs following transfection, cells were trypsinized and seeded in 8-chamber slide (Ibidi #80826) at a density of 20,000/well and allowed to attach. The following day, proliferation media was replaced with the appropriate treatment condition and imaging commenced (time of initiation and duration are shown in figure legends).

Microscopy

Spinning-disc confocal microscopy: Live cell imaging (37° C., with 5% CO₂) was performed using Olympus IX83 fluorescence microscope controlled via VisiView software (Visitron Systems GmbH) and equipped with CoolLED pE-4000 light source (CoolLED Ltd., UK), an PLAPON60XOSC2 NA 1.4 oil immersion objective, and a Prime 95B sCMOS camera (Photometrics). Fluorescence excitation and emission were detected using filter-sets 488 nm and 525/50 nm for GFP, 561 nm and 609/54 nm for mCherry.

Cell Discoverer 7-Zeiss: Fixed samples (FIG. 1B) were imaged using Cell discoverer 7-Zaiss inverted in widefield mode with s CMOS 702 camera Carl Zeiss Ltd. Images were acquired using a ZEISS Plan-APOCHROMAT 20×/0.95 Autocorr Objective. ZEN blue software 3.1 was used for image acquisition using AF647 for the acquisition of the MyHC signal and DAPI for the nuclei. If necessary, linear adjustments to brightness and contrast were applied using ImageJ v1.52 software (Schneider et al., 2012).

Nikon Eclipse Ti2 microscope: Fixed samples (FIGS. 2A-J and FIGS. 3A-M) were imaged using the Nikon Eclipse Ti2 microscope and NIS-Elements imaging software ver.5.11.00. using a 10× objective for the acquisition of MyHC, MYOG, KI-67, pH3 and DAPI staining. If necessary, linear adjustment to brightness and contrast were applied using Photoshop. Live-imaging of tdTomato expressing myoblasts (not shown) were imaged using the Nikon Eclipse Ti2 microscope and NIS-elements software, using a 10× objective. linear adjustments to brightness and contrast were applied using ImageJ v1.52 software (Schneider et al., 2012).

Quantification of fusion index. Following immunostaining and imaging, a fusion index was quantified by manually identifying nuclei found in a MyHC positive cell with at least 2 nuclei. Then the values were expressed as a percentage of the total nuclei per field. Briefly, in Figures where fusion index is stratified into subgroups of fiber size, the nuclei number in MyHC positive cell was manually quantified in a given field and stratified into groups of mononucleated, bi-nucleated myotubes, myotubes with 3-10 nuclei and myotubes with greater than 10 nuclei. For myotube growth curves, LifeAct-EGFP; nTnG reporter primary myoblasts underwent time-lapse imaging beginning at 8 hours after treatment and followed until 23 hours. Fields were analyzed hourly, and nuclei per cell was quantified and stratified into mononucleated, bi-nucleated, tri-nucleated and cells with ≥4 nuclei.

Data-driven cell fusion simulations. For each experiment we defined a matched “shadow” simulation that compared the experimental fusion dynamics to a scenario where cell-cell fusion occurred randomly. The input for the “shadow” simulation was the observed distribution of multinucleated cells in each time frame. This included the number of cells with a single, pair, triplet or quartette-or-more nuclei that were manually annotated with a time resolution of 60 minutes intervals between consecutive measurements. The estimated number of fusion events per time interval was calculated as the difference between the weighted accumulated number of multinucleated cells Σᵢ₌₂ⁱ⁼⁴[(Cₓ(t)−Cₜ₋₁(i)*(i−1)], where i is the number of nuclei in a multinucleated cell, t is the time interval and Ct(i) is the number of cells with i nuclei at time interval t. We assumed that the number of cells remain constant throughout the experiment. The input for the simulation included (1) N—the number of nuclei determined at the onset of the experiment, where each of the cells had exactly one nucleus. And (2) N_fusion—the list of estimated fusion events per time interval. For each time interval t, we simulated N_fusion(t) fusion events by randomly selecting two cells and fusing them, generating one cell with the joint number of nuclei for the next simulation round. For each time interval, we recorded the probability of a nucleus to be part of a 4-nuclei cell, i.e., what is the fraction of nuclei in a multinucleated cell that contains 4 or more nuclei. This fraction was used as a measure to compare experiments to simulations. Due to annotation limitations, we considered multinucleated cells that contained 4 nuclei. This means that a multinucleated cell with more than 4 nuclei was annotated as a 4-nuclei cell. On the one hand, this limitation had implications in the calculations of the estimated number of fusions—which was a lower bound to the true number of fusion events. On the other hand, the calculated probability for a nucleus to take part in a 4-nucleated cell was also a lower bound to the true probability. This double lower bound effect is expected to cancel each other and also takes place only in the later stages of an experiment.

Statistical significance for each experiment was calculated using a Bootstrapping approach. For each experiment we performed 1000 simulations. For each time interval in each simulation, we recorded whether the probability of a nucleus to be in a 4-multinucleated cell was equal or exceeded the experimental observation. The p-value was defined as the probability for a simulation to exceed the experiment with this measure. We used a cutoff threshold ≤0.05 (50 simulations out of 1000 for each experiment) to reject the null hypothesis of random fusions. Importantly, this assessment provides a p-value for each time interval in each experiment. As a more realistic scenario we considered the possibility that the probability of selecting a cell for fusion was proportional to the number of nuclei within it. This followed the simplistic assumption that the area of an n-nucleated cell is n times the size of a single-nucleated cell. Thus, simulating the situation where a cell fuses randomly, but its chance of bumping-and-fusing into another cell is dependent on its area.

Quantitative real-time PCR (qRT-PCR). Total RNA was isolated using Tri-Reagent (SIGMA) according to the manufacturer's instructions. cDNA was synthesized with the High Capacity cDNA Reverse Transcription Kit (Applied Biosystems according to the manufacturer's instructions. qRT-PCR was performed with SYBR green PCR Master Mix (Applied Biosystems) using the StepOnePlus Real-time PCR system (Applied Biosystems). Values for specific genes were normalize to either Gapdh or Hprt housekeeping control as indicated in Figure legend. Expression was calculated using the ddCT method.

Western Blot analysis. Cultured cells and whole tissues extracts were prepared with RIPA buffer supplemented with protease inhibitor cocktail (SIGMA P8340), and phosphatase inhibitor cocktails (SIGMA P5726 and P0044). Western blotting was performed using the Mini-PROTEAN Tetra Cell electrophoresis system, and transferred to PVDF membranes. The following primary antibodies concentrations were used p-CAMKII 1:1000 (Abcam ab182647), CaMKII 1:1000 (Cell Signaling 3362), GAPDH 1:10,000 (Abcam ab181602), p-ERK1/2 1:20,000 (SIGMA M9692), ERK1/2 1:40,000 (SIGMA), Myosin Heavy Chain (MF-20 DSHB hybridoma supernatant 1:100) Horseradish peroxidase conjugated secondary anti-mouse, anti-rabbit or anti-goat was used to detect proteins (Jackson Immunology). Western blots were imaged using the Chemidoc Multiplex system (Bio-rad).

Co-immunoprecipitation (Co-IP). Primary myoblasts derived from gastrocnemius muscle were pooled from 10 mice and plated on 15 cm dishes and allowed to adhere for 24 hours. The following day, Bio-Amf2 media was replaced supplemented either with DMSO or 1 uM SCH772984. Cells were treated for 4 hours, and then nuclear lysates were prepared according to the instructions of the Universal Magnetic Co-IP KIT (Active Motif cat #54002). 1 mg of protein was used to immunoprecipitate ERK1/2 using 2 ug of ERK1/2Antibody (Sigma M7927). Rabbit IgG was used as a control. Reactions were resuspended in 2× Sample buffer with DTT and loaded onto a 12% Tris-glycine SDS-page gel. 1% of original volume of lysate loaded into IP reaction was loaded into the gel as input control. Membranes were blotted with RXR antibody (SCBT sc-553).

Cloning and expression of CaMKII adenovirus for fusion assay. CaMKII-6 cDNA was PCR amplified from mouse primary myoblasts using primers, CAMK2D-F and CAMK2D-R, designed against published CaMKII-6 sequences, and ligated into the PGEM-T-easy cloning system (Promega), and sequence validated. The T287V mutation was introduced by PCR assembly. A 909 bp upstream PCR fragment was amplified with primer sequences designed to incorporate a Xhol site and FLAG tag at the N-terminus of CAMK2D and a the T287V mutation, using primers Xhol-FLAG-CAMK2D-F and CAMK2D-T287V-IN-R. The 640 bp downstream PCR fragment was similarly amplified with a primer to introduce the T287V mutation and a BamHI site using the primers CAMK2D-T287V-IN-F: and CAMK2D-BamHI-R. Both PCR fragments were used as template for an assembly PCR reaction with Xhol-FLAG-CAMK2D-F and CAMK2D-BamHI-R primers to generate a 1525 bp product, which was ligated back into PGEM. Similarly, the WT CAMK2D was amplified with the same primers to incorporate the FLAG-tag and ligated back into PGEM. The 1525 bp FLAG-CAMK2D^{WT} and FLAG-CAMK2D^{T287V} fragments were digested out of PGEM with BaMHI and Xhol and ligated into pEGFP-C1 (Clontech). A 2865 bp product EGFP-FLAG-CAMK2D^{WT} or EGFP-FLAG-CAMK2D^{T287V} was digested out using KPNI and ECORV and inserted into RedTrackCMV (addgene plasmid #50957). RedTrack-CMV-EGFP-FLAG-CAMK2D^{WT} (Ad-CaMK2D^{WT}), RedTrack-CMV-EGFP-FLAG-CAMK2D^{T287V} (Ad-CaMK2D T287), and empty RedTrack-CMV (Ad-Ctrl), vector were used as template to grow adenovirus using the Adeasy system as previously described (Luo et al., 2007). Myoblasts were infected with crude adenoviral lysate at an MOI of 100 at the time of plating (reverse infection) in BioAmf2 media. Following overnight incubation, the cells were washed once with warm DM and were incubated for 72 hours in DM and number of nuclei per fiber was quantified.

Myomaker plasmid construct and overexpression fusion assay. The pBabe-puro and pBabe-GFPfarn plasmids were purchased from Addgene (Plasmid #1764 and #21836, respectively). The pBabe-CFPn1s was constructed by replacing the Puro R from the pBabe-Puro, with DNA sequence encoding CFP fused to two tandem repeats of a nuclear import signal. pBabe-dsRed plasmid was constructed in a similar manner. To generate pBabe-Mymk-CFPn1s, the CDS sequence of murine MYMK (Millay et al., 2013) was subcloned in the MCS region of pBabe-CFPn1s plasmid using restriction free cloning. Retroviruses were generated as described above. Myoblasts were seeded at 7×10³ per well of 96 well. The following morning cells were infected with viral prep supernatants together with polybrene (6 ng/μL) for 1 hour, then replaced with fresh growth media, then after 8 hours the media was changed according to indicated conditions. Cells were fixed and stained at 18 hours post treatment.

CTX induced injuries. All experiments were approved by the Animal Care and Use Committee of the Weizmann Institute of Science (IACUC application #00720120-4). Pax7^{CreERT/+}; CaMK2δ^{fl/fl}/γ^{fl/fl} (scDKO) or Pax7^{+/+}; CaMK2δ^{fl/fl}/γ^{fl/fl} (WT) received intraperitoneal tamoxifen administration beginning at weaning (4 weeks of age) for 6 consecutive days, followed by weekly boosters until 12 weeks of age. Mice were anesthetized with isoflurane and injected in the right gastrocnemius muscle with CTX dissolved in PBS (latoxan) at 10 sites (3 ul per site) at 10 uM, using a Hamilton syringe. All injuries were performed on female mice. For mice that received a repeat injury: following the first injury, mice were maintained for an additional 8 weeks and then injured again in the right gastrocnemius, as described above.

Histology and CSA quantification. 14 days post CTX induced reinjury, muscles were excised and fixed in 4% PFA, embedded in paraffin, and sectioned. Muscles were cut transversely in the center and cut into serial sections at 0.3 mm intervals. For analysis of muscle fiber cross-sectional area (CSA), sections were permeabilized and stained with WGA and DAPI. The entire muscle transverse section of WT and scDKO mice taken at identical locations within the muscle were imaged using the Nikon at 10×. CSA was quantified using the Open-CSAM, semi-automated analysis tool with FIJI (Desgeorges et al., 2019). Each field was evaluated for accuracy and manually corrected. At least 9,000 fibers/mouse were measured.

Statistics. Sample size was chosen empirically following previous experience in the assessment of experimental variability. Generally, all experiments were carried out with n≥3 biological replicates. The analyzed animal numbers or cells per groups are described in the respective figure legends. All animals were matched by age and gender, and cells harvested from mice of similar age. Animals were genotyped before and after completion of the experiment and were caged together and treated in the same way. Statistical analysis was carried out using Prism software. Whenever comparing between two conditions, data was analyzed with two tailed student's t-test. If comparing more than two conditions, ANOVA analysis with multiple comparisons was executed. In all Figures, measurements are reported as mean of multiple biological repeats, and the error bars denote SEM, unless otherwise specified in the figure legend. Throughout the study, threshold for statistical significance was considered for p-values<0.05, denoted by one asterisk (*), two (**) if P≤0.01, three (***) if P<0.001 and four (****) if P≤0.001.

### Example 1

### ERK1/2 Inhibition Induces Myoblast Differentiation and Hyper-Fusion

The present inventors hypothesized that ERK1/2 prevents myogenesis not only through maintenance of myoblast proliferation but also through the active repression of pro-myogenic processes, by inhibiting gene expression through various nuclear targets (Michailovici et al., 2014; Yohe et al., 2018). In order to examine the role of ERK1/2 in myoblast differentiation and fusion, the specific ERK1/2 inhibitor SCH772984 (ERKi) was applied to first passage mouse-derived primary myoblasts in growth medium, and resulted in the robust formation of myotubes (FIG. 1A-B) as compared to conventional serum-reduced differentiation medium (DM) (90.5% in ERKi after 24 hours vs. 11.6% in DM). The differentiation and fusion factors MyoD, MyoG, Mymk, and Mymx were upregulated and the fraction of MYOG positive nuclei was significantly higher at 24 hours in cells treated with ERKi compared to DM (FIG. 1C-E). Moreover, immunofluorescence staining of ERKi treated cultures with the proliferation markers KI-67 (FIGS. 1F and 1G) and phosphorylated Histone 3 (pH3, FIGS. 1H and 1I), demonstrated that myoblasts undergo cell-cycle arrest. Taken together, these results suggest that ERKi induces a more robust differentiation and fusion response as compared to myoblasts cultured in common DM, leading to hypertrophic myotubes.

### Example 2

### ERK1/2 Inhibition Initiates an RXR/RYR-Dependent Fusion Response

The present inventors hypothesized that ERK1/2 represses a downstream target, which drives the fusion process leading to myofiber growth. In cancer cell lines, ERK1/2 phosphorylates RXR (nuclear retinoid-X receptor), leading to inhibition of its transactivation potential (Macoritto et al., 2008; Matsushima-Nishiwaki et al., 2001). RXR activity promotes myogenesis mainly through regulation of Myod expression and as a MYOG co-factor (Alric et al., 1998; Froeschlé et al., 1998; Khilji et al., 2020; Le May et al., 2011; Zhu et al., 2009). Thus, it was hypothesized that RXR is a nuclear ERK1/2 target in myoblasts. RXR immunoprecipitated with ERK1/2 in myoblasts, and this interaction was attenuated upon treatment with ERKi (FIG. 2A). Additionally, co-treatment of myoblasts with ERKi and the specific RXR antagonist HX531 (20 uM, RXRi) suppressed fusion by 47% at 24 hours (FIGS. 2B and 2C), without affecting differentiation, as measured by the percent of nuclei which stained positive for MYOG (FIGS. 2B and 2D). These results suggest that upon ERK1/2 inhibition, RXR becomes activated and promotes myoblast fusion.

Next, it was shown that ERKi-treated myoblast cultures upregulated the expression of Ryr1 and Ryr3, as well as the Ca²⁺ sensing channels such as SERCA1/2 (Atp2a1 and Atp2a2) and Orail 12 and STIM1I2 (FIG. 2E). Interestingly, co-treatment of myoblasts with ERKi and RXRi resulted in the downregulation of Ryr1 and Ryr3 mRNA expression 24 hours post treatment (FIG. 2F). Ryanodine receptors (RYR1-3) are channels which mediate release of Ca²⁺ stores from the SR into the cytoplasm during excitation-contraction coupling in both cardiac and skeletal muscle cells. However, since myoblast fusion precedes muscle-contraction in the hierarchy of molecular events, we wondered whether elevated cytosolic Ca²⁺ levels have a role in myoblast fusion as previously suggested (Shainberg et al., 1969). Co-treatment of cultures with ERKi and the RYR specific antagonist Dantrolene (50 uM, RYRi) reduced fusion by 60% (FIGS. 2B and 2G) without affecting differentiation, measured by MYOG staining (FIGS. 2B and 2H). Along the same line, myoblasts co-treated with ERKi and the calcium chelator BAPTA-AM exhibited reduced fusion by 81% (FIGS. 2B and 2I), without affecting early differentiation (FIGS. 2B and 2J). Taken together, these results demonstrate that Ca²⁺ is essential for myoblast fusion and that upon ERK1/2 inhibition, RXR-transactivates Ryr1 and Ryr3 expression, which likely promotes Ca²⁺ release from the SR, resulting in myofiber fusion and growth.

### Example 3

### Calcium-Dependent CaMKII Activation Promotes Myoblast Fusion with the Growing Myotube

Next, it was determined if Ca²⁺-dependent phosphorylation and activation of cellular kinases might be involved in the observed myofiber growth via myoblast fusion. By monitoring the levels of the Ca²⁺-dependent enzyme p-CaMKII T287, it was found that CaMKII is activated upon treatment of myoblasts with ERKi (FIG. 3A), and that its activation is dependent on the upstream activity of RXR, RYRs and Ca²⁺ (FIG. 3B-D). Strikingly, co-treatment with the CaMKII inhibitor KN93 (5 uM; CaMKIIi) suppressed CaMKII activation (FIG. 7A) and the formation of polynucleated myotubes, while maintaining bi- and tri-nucleated MyHC+ cells (FIGS. 3E and 3F). Co-treatment of ERKi with CaMKIIi did not affect cell cycle arrest as measured by pH3 staining (FIG. 7B) or expression of the cell-cycle inhibitors p21 and p27, compared to ERKi alone (FIG. 3H). Similarly, co-treatment with CaMKIIi did not affect the initiation of the myogenic program, as both the percentage of MYOG positive nuclei (FIGS. 3E and 3G) and the expression of differentiation markers remained unaffected (FIG. 3H). Cell motility upon co-treatment with ERKi and CaMKIIi was unchanged compared to ERKi-treated cells, demonstrating that fusion failure is not due to an effect on cell migration (not shown). These results suggest that CaMKII activation is essential for myoblast to myotube fusion but not for myoblast-to-myoblast fusion. Therefore, bi- and tri-nucleated myotubes form in the presence of CaMKIIi but these fail to expand into large multinucleated fibers.

The expression of both Mymk and Mymx was elevated upon treatment with ERKi; however, only the elevation of Mymk expression, but not of Mymx, was partially suppressed upon co-treatment with ERKi and CaMKIIi (FIG. 3H). It was therefore examined whether reduced fusion upon CaMKII inhibition could be attributed to decreased Mymk expression. To assess this, MYMK was over-expressed by retroviral transduction in primary myoblasts and subjected to treatment with ERKi and CaMKIIi. It was found that ERKi-dependent fusion was enhanced upon overexpression of MYMK as the average number of nuclei per myotube was nearly doubled. Interestingly, this effect was completely dependent on CaMKII activity as large myotubes were lost upon co-treatment with CaMKIIi and the accumulation of mono-nucleated, and bi-nucleated cells was similar to that of cells transduced with control retrovirus. These experiments show that CaMKII functions either downstream or in parallel to MYMK.

To examine whether CaMKII activation is sufficient to induce myoblast-to-myotube fusion, primary myoblasts were transduced with either empty adenovirus vector (Ad-Ctrl), Ad-CaMK2δ^{WT}, or phospho-null Ad-CAMK2δ^{T287V}, and induced to differentiate in DM for 72 hours. It was found that exogenous CAMK2δ^{WT} was activated by phosphorylation in DM, yet CAMK2δ^{T287V} failed to undergo activation (FIG. 3I). Moreover, it was observed that while expression of CAMK2δ^{WT} enhanced formation of bi- and poly-nucleated MyHC positive cells, expression of CAMK2δ^{T287V} did not, but rather suppressed growth of multi-nucleated cells compared to the control (FIG. 3J). This result suggests that CaMKII activation is sufficient to promote secondary (myoblast-to myotube) fusion.

Activation of CaMKII is a late event occurring by 16 hours post ERKi treatment, coinciding with an elevation in MyHC and MEF2C levels (FIG. 3K). Interestingly, both RYRs and activated CaMKII are primarily localized to myotubes rather than to mono-nucleated MyHC+ cells, following ERK inhibition (FIGS. 3L and 3M). Taken together these results suggest that Ca²⁺-dependent CaMKII activation is a downstream event to the activation of RXR and RYR, and that CaMKII activity is essential in myotubes for their expansion by mediating myoblast-to-myotube fusion.

### Example 4

### Myotubes Grow Asymmetrically Through Recruitment of Mono-Nucleated Myoblasts at a Fusogenic Synapse

As the present inventors observed that cotreatment of ERKi with CaMKIIi did not exhibit a total loss of fusion, and moreover, that RYR and activated CaMKII proteins were exclusively located in myotubes and not in differentiated MyHC+ myoblasts under ERKi treatment, it was hypothesized whether myoblast fusion occurs predominantly between mononucleated myoblasts and myofibers as previously described in Drosophila (Önel and Renkawitz-Pohl, 2009). To explore this notion further, live-cell imaging was performed with calculated hourly fusion index for a period of 8-23 hours post-treatment with ERKi. It was found that after the initial formation of bi- and tri-nucleated cells, these cells expand rapidly by growing at the expense of the mononucleated cells (FIG. 4A and not shown). To verify that the expansion of the fibers is a regulated phenomenon, data driven simulation was performed. The present inventors considered the higher probability of the larger, multinucleated cells to interact and fuse with their neighbors, suggesting that myotubes attract neighboring myoblasts to fuse (FIG. 4B). None of the simulations recapitulate the present results, implying that fiber growth is a regulated process.

This behavior was also apparent in high resolution time-lapse microscopy of myoblasts expressing a membrane-targeted GFP and cytoplasmic dsRed. Fibers were shown expanding rapidly through several fusion events, which occurred nearly simultaneously (FIG. 4C and not shown). The present inventors also observed that myoblasts display concerted collective movement and increased actin-rich membrane protrusions after ERKi treatment (not shown). Moreover, live-cell imaging revealed that fusion occurs at protrusions extending from only one of the fusing partners (observed in 85% of fusion events; n=46; FIG. 4D and not shown). To visualize Ca²⁺ dynamics during ERKi-induced myogenesis, imaged myoblasts were harvested from GCaMP6 Ca²⁺ reporter mice. These experiments revealed that a pulse of Ca²⁺ in nascent myotubes precedes the phase of rapid myotube growth, supporting the notion that Ca²⁺ released from the SR in early myotubes is responsible for CamKII activation, which mediates an asymmetric fusion reaction between the myoblasts and myotubes (FIG. 4E and not shown). Taken together, these results suggest that myoblast fusion in mammals is initiated by the generation of multinucleated founder cells (2-3 nuclei) that expand by fusion of the “attacking” myoblasts to the “receiving” myotube, and that this phenomenon is mediated by CaMKII activity in myotubes.

### Example 5

### CaMKII is Required for Efficient Muscle Regeneration

To examine the role of CaMKII during muscle regeneration in vivo, wildtype mice were subjected to cardiotoxin (CTX) induced injuries, and tissues were collected on the day of injection, and consecutively on days 2-8 post injury. An acute activation of ERK1/2 was evident two days post CTX injury, likely associated with an increased proliferation of myoblasts (FIG. 5A). By the third day post injury, levels of CaMKII increased in regenerating muscle and remained elevated throughout the 8 days examined; this was accompanied by a peak in CaMKII activation at 5 days post injury (FIG. 5A). Following these promising results, the present inventors sought to examine the requirement for CaMKII during muscle regeneration. To accomplish this, the present inventors generated a tamoxifen inducible and satellite cell specific conditional double knockout mouse of the CaMKII δ and γ isoforms (FIGS. 5B and 5C).

It was found that CaMKII protein levels in quiescent satellite cells are highly stable and not efficiently reduced even 3 months following tamoxifen administration. To overcome this issue, the present inventors implemented a repeat injury model. It was reasoned that an initial round of regeneration would be needed to substantially reduce the levels of the highly stable CaMKII protein in the satellite cell pool (in order to assess function in vitro), as well as allow for partial knockdown in the regenerated muscle fibers, as the DNA content of the fusing myoblasts would now be integrated. Hence, at four weeks old, satellite-cell double knockout (scDKO) Pax7^{CreERT/+}; CaMK2δ^{fl/fl}/γ^{fl/fl} or Pax7^{+/+}; CaMK2δ^{fl/fl}/γ^{fl/fl} (WT) mice were given tamoxifen to induce Cre/Lox based gene disruption. When the mice were twelve weeks of age, cardiotoxin (CTX) was administered, and the mice were allowed to fully regenerate for 8 weeks. At 8 weeks post injury, mice were either sacrificed to harvest primary myoblasts from the injured leg (to assess function in vitro), or subjected to a second CTX injury, and sacrificed 14 days post injury for histological analysis. Reduction in CaMKII levels were indeed validated in scDKO myoblasts harvested 8 weeks following the first injury (FIG. 5D). A fusion index demonstrated that such scDKO myoblasts exhibited a significant defect in ERKi-induced fusion compared to those isolated from their wildtype littermates (FIGS. 5E and 5F). Specifically, scDKO myoblasts exhibited a loss of the hyperfused myotubes observed in the WT cultures, and instead accumulated mono-nucleated MyHC⁺ cells and nascent myotubes (FIGS. 5E and 5F). These results match and recapitulate the observations made on myoblast cultures treated with CaMKIIi.

Furthermore, scDKO mice which received repeated injuries, had significantly smaller fiber cross-sectional area (851.411M²±37.5) compared to their WT counterparts (97511M²±25) (FIGS. 5G and 5H), and a trend towards more centrally located nuclei (FIG. 51). Taken together, the genetic loss of CaMK2δ/γ is sufficient to impair myoblast fusion and muscle regeneration. Overall, and without being bound by theory, the present inventors have described a signaling pathway leading to activation of CaMKII, which mediates myotube driven asymmetric myoblast fusion. Upon ERK1/2 inhibition, myoblast proliferation is arrested, and the differentiation program is initiated. ERK1/2 inhibition results in RXR activation and induction of RYR expression in nascent myotubes, leading to Ca²⁺-dependent activation of CaMKII in the myotube, and ultimately CaMKII-dependent asymmetric myoblast-to-myotube fusion (FIG. 6A-6C).

### Example 6

### ERK Inhibition Improves Maturation of Muscle Fibers from Mouse Myoblasts

First passage primary mouse derived myoblast were seeded in proliferation media, in equal number in 12-well tissue culture plates that were precoated with 10% Matrigel solution. Following 24 hours to allow for complete attachment, culture media was washed and replaced either with fresh proliferation media (PM), PM supplemented with 1 uM SCH 772984 (ERKi) or differentiation media (DM). After 24 hrs, cells were lysed and RNA was collected. Gene expression analysis was carried out using SYBR green qRT-PCR analysis.

Results

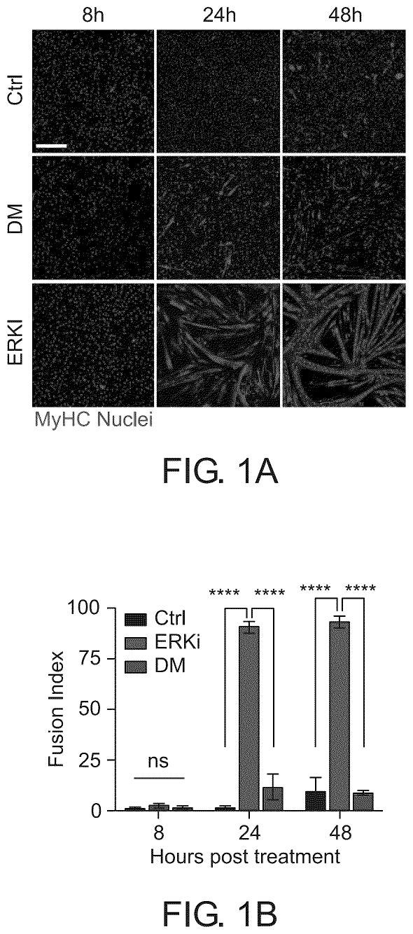

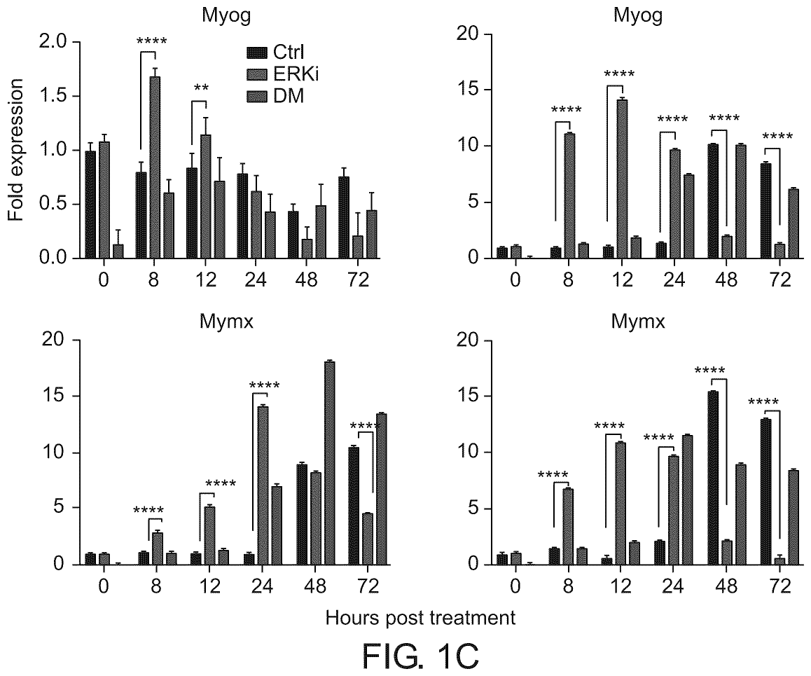

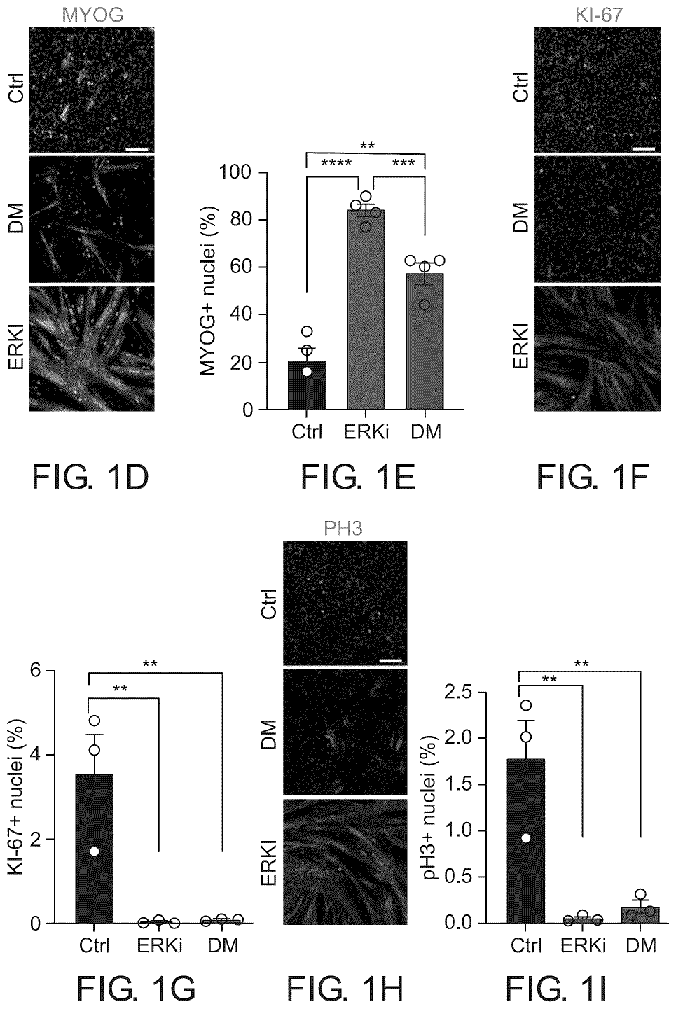

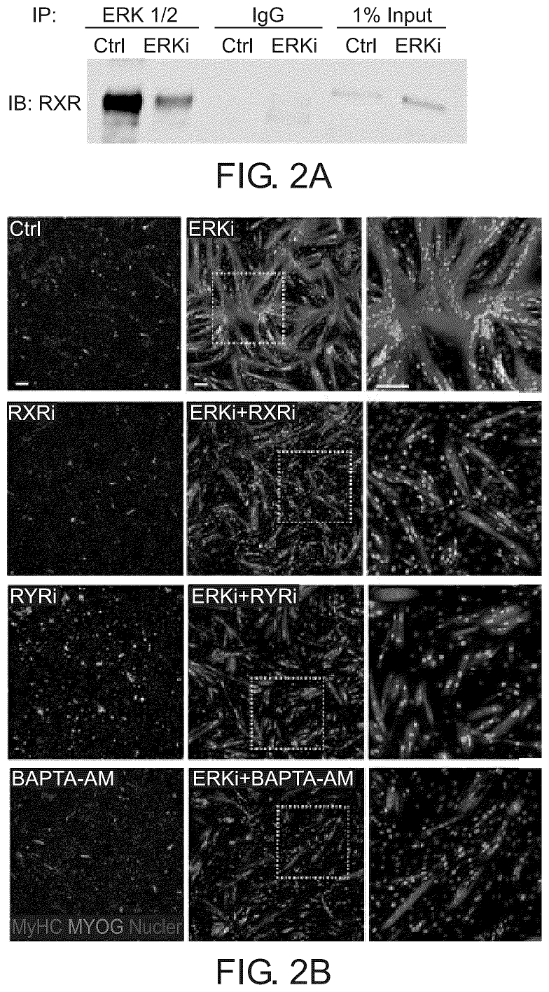

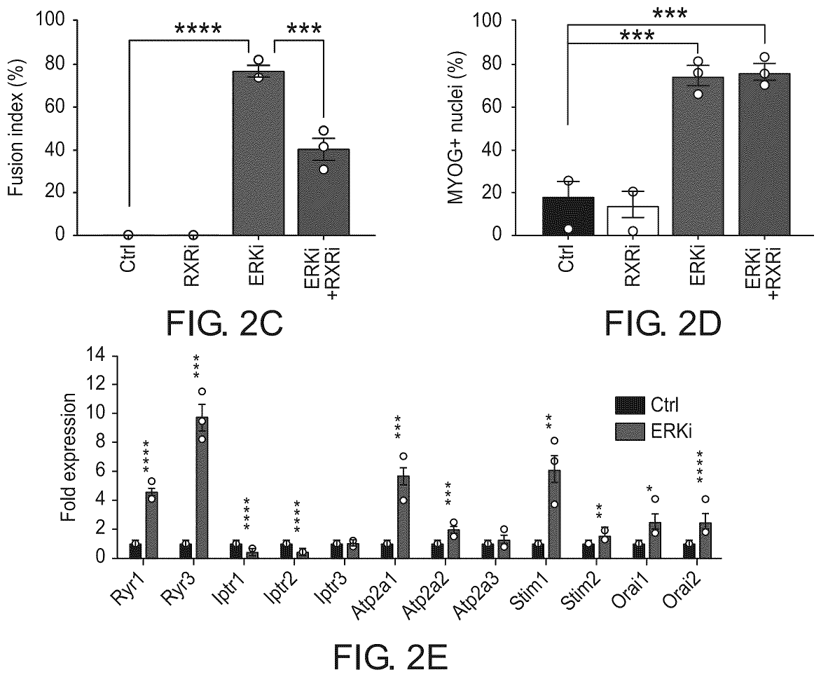

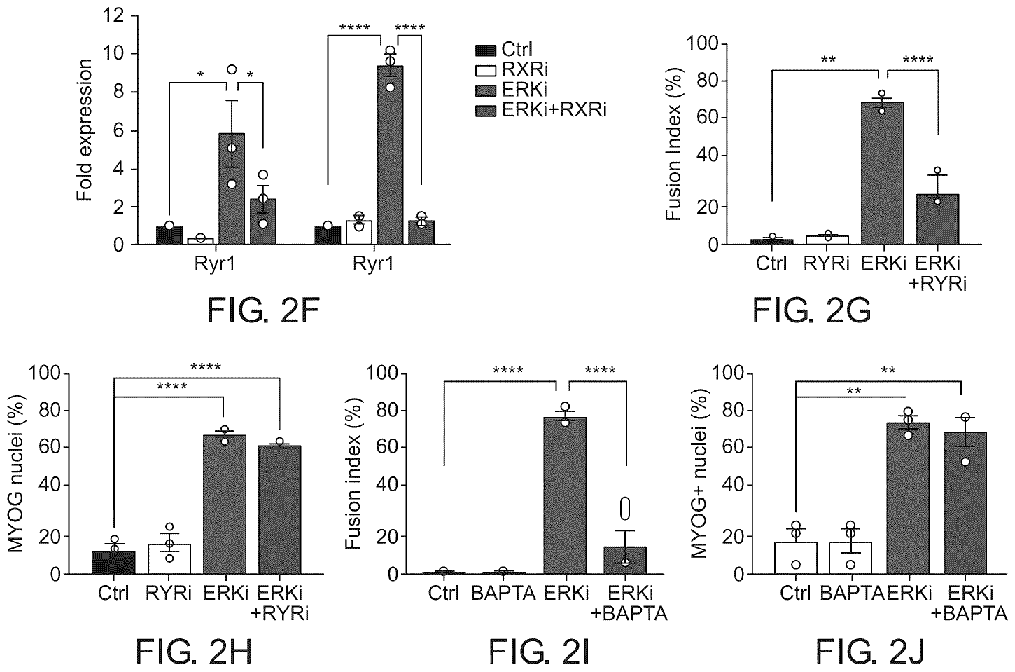

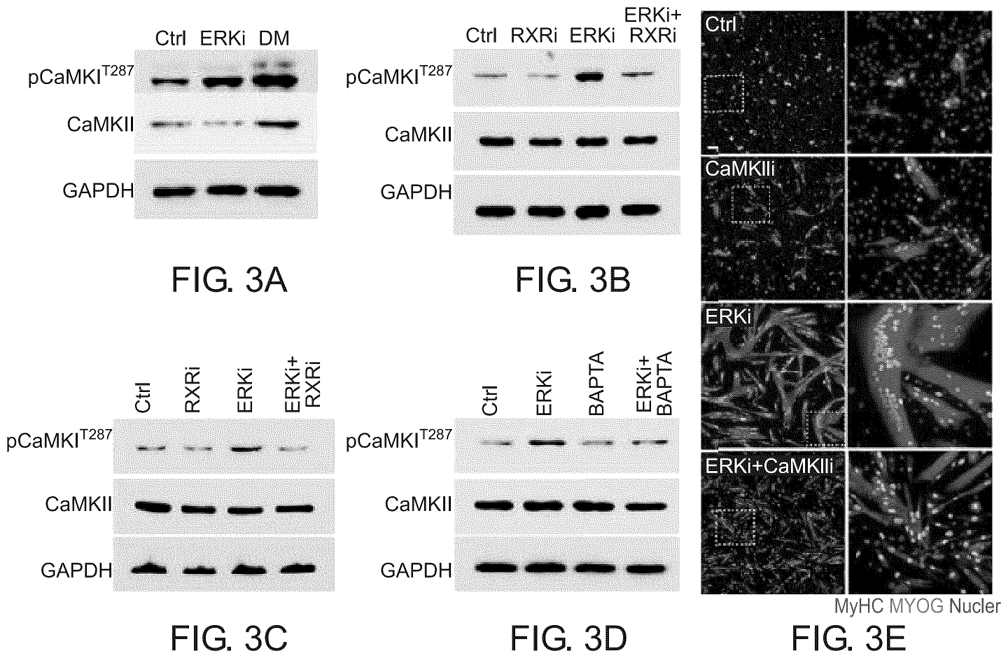

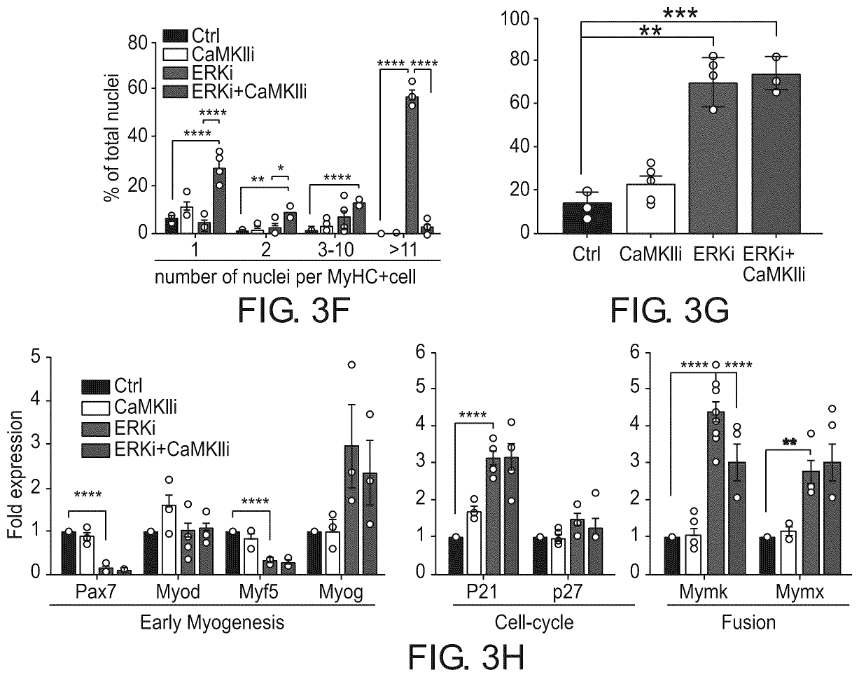

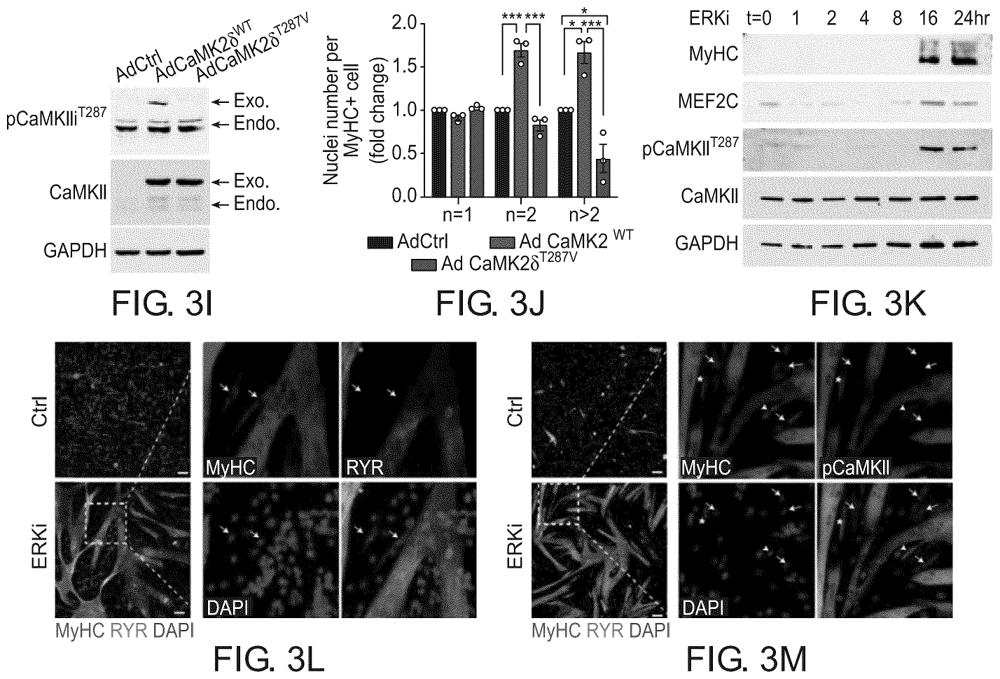

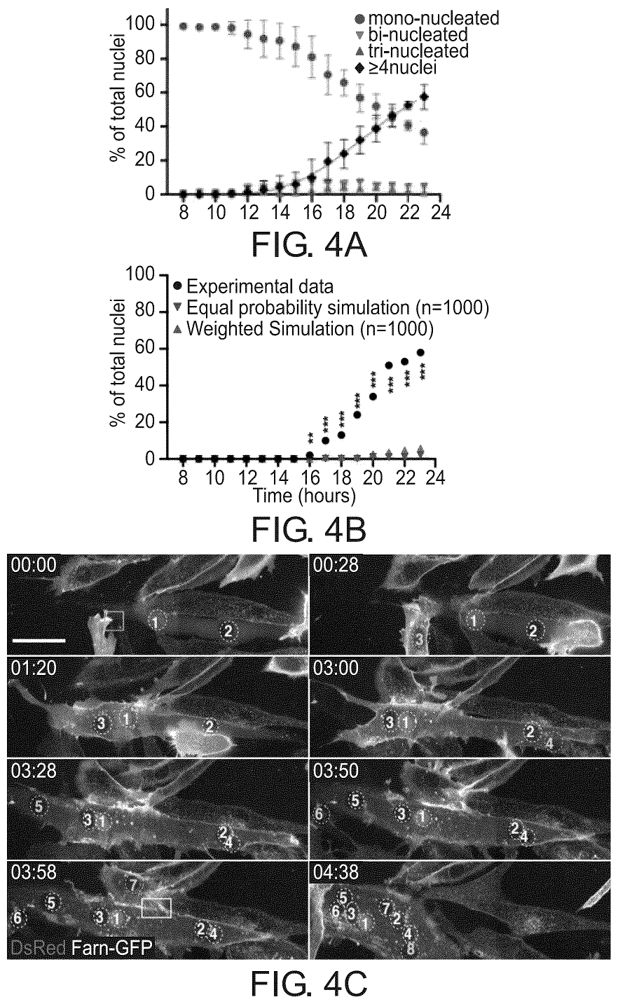

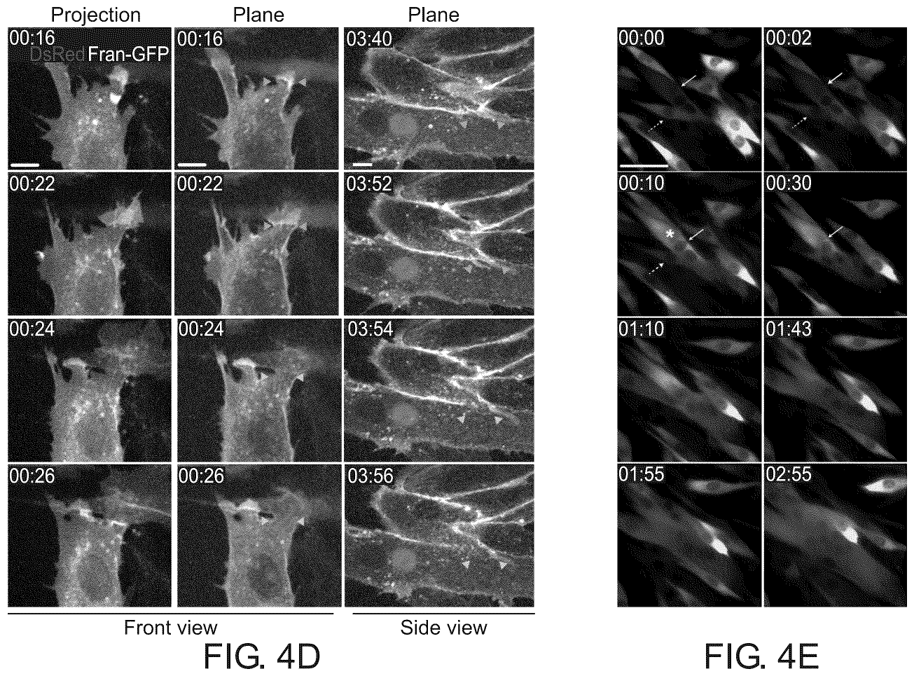

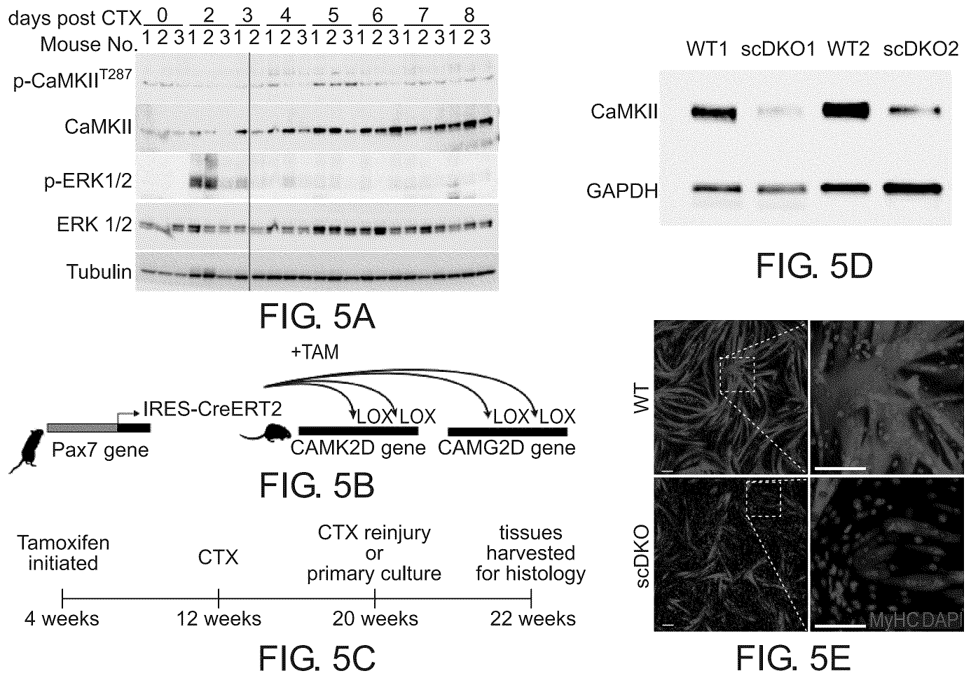

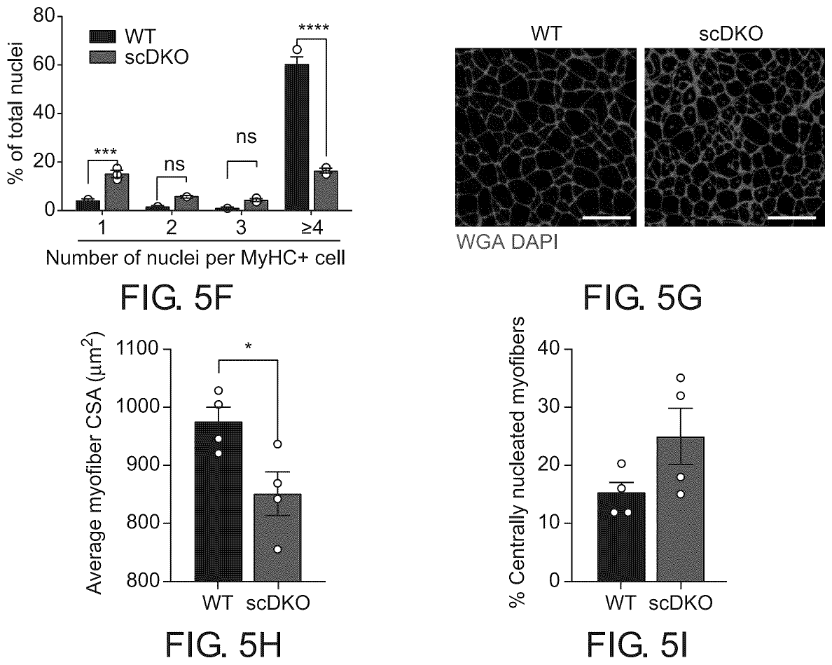

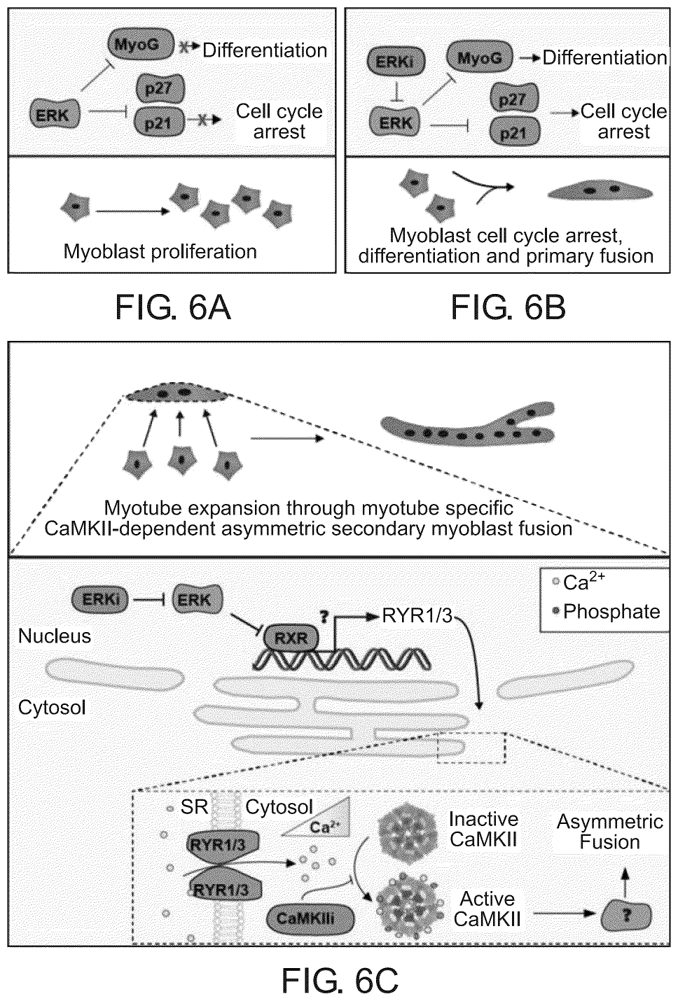

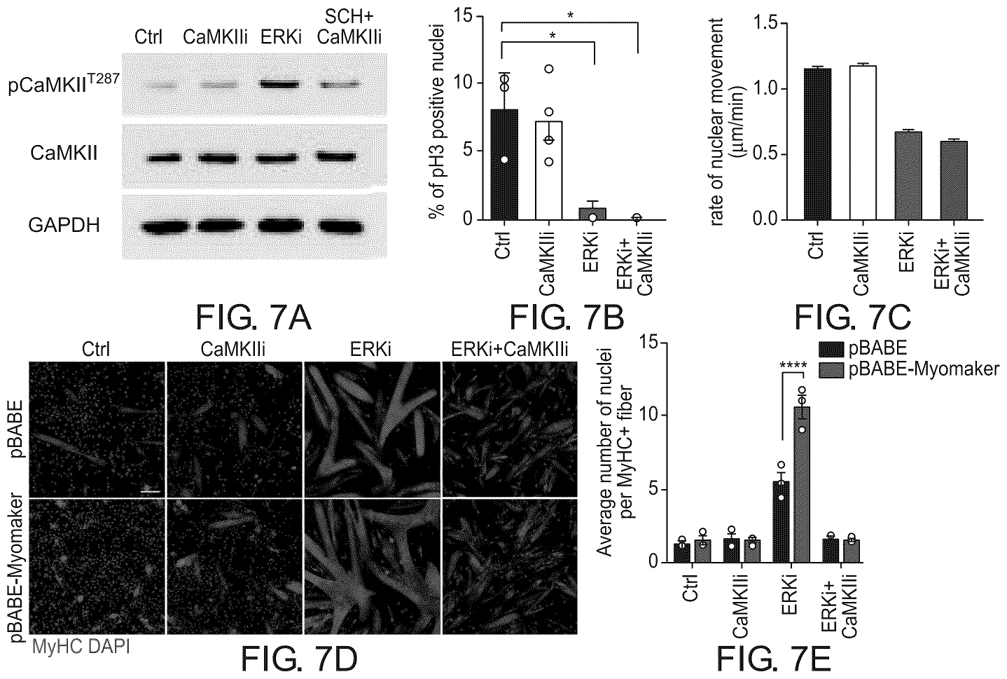

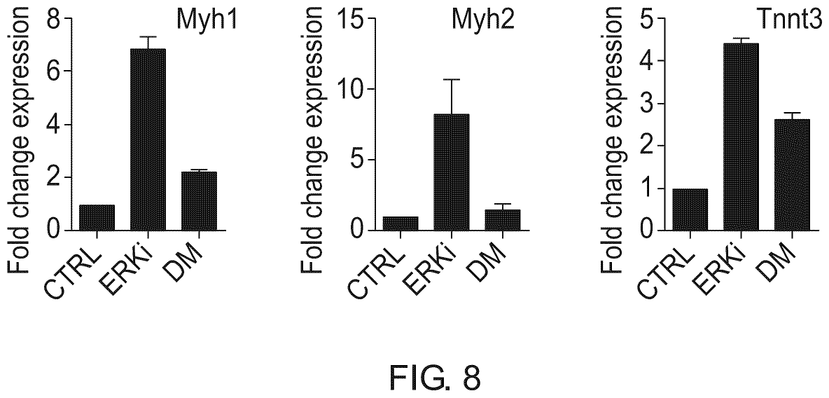

As shown in FIG. 8, ERKi-induced mouse myotubes have stronger expression of the maturation markers myosin heavy chain and troponin, components of the sarcomeric machinery necessary for muscle contraction as compared to DM, suggesting that it is possible that ERKi induced fibers reach maturation earlier and may have the ability to contract prior to those fibers obtained using DM.

### Example 7

### ERK Inhibition Promotes Early Induction of Differentiation, Fusion, and Myotube Formation in Chicken Myoblasts

Chicken primary derived myoblasts were used as a model to evaluate the efficiency of SCH772984 as the ERKi, compared to conventional differentiation medium (DM) for the purpose of producing muscle (meat) in tissue culture conditions. Chicken myoblasts were isolated from chicken embryos and expanded in proliferation medium. When sufficient cell numbers were acquired, cells were seeded on tissue culture plates and allowed to adhere for 24 hours. Then medium was changed to either PM supplemented with ERKi or DM. At 24-hours post treatment, the cells that received ERKi were replenished with fresh PM (without the addition of more ERKi), and DM treated cells were replenished with fresh DM. Media was replaced daily over a period of 72 hours.

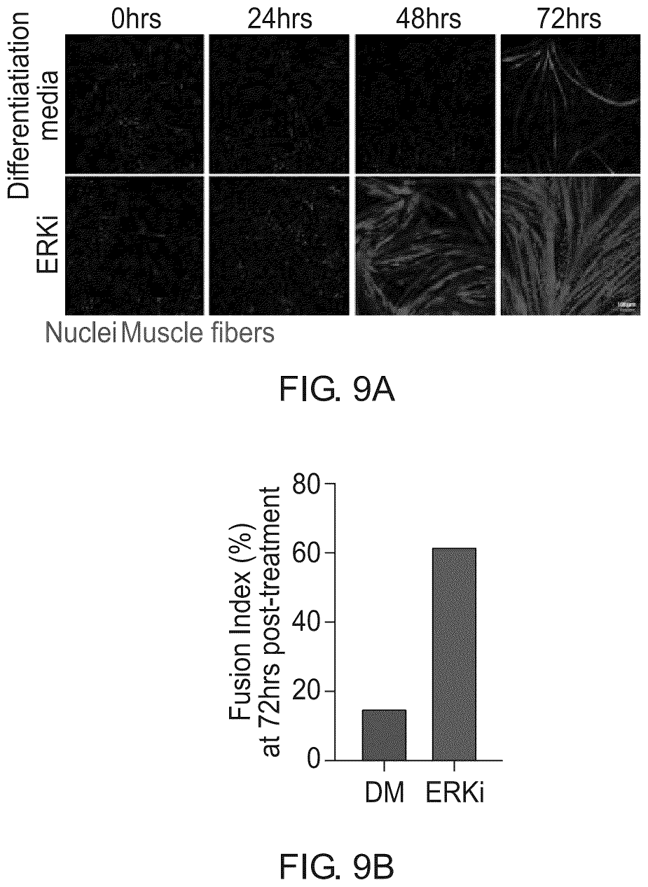

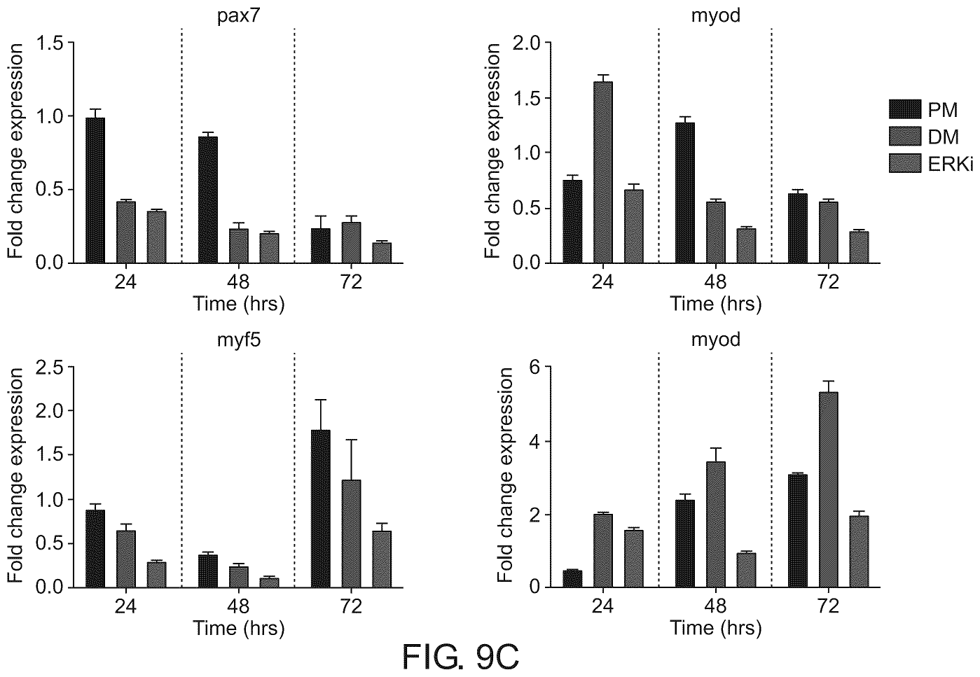

Evaluation of muscle fiber formation was achieved by fixing the cells and staining for expression of Myosin Heavy Chain (FIG. 9A). The time-course demonstrated the significant enhancement of fiber formation following treatment with ERKi; early myotubes consisting of 2-3 nuclei are apparent by 24-hrs post treatment which continue to grow throughout the remainder of the 72-hour time-course. Yet, treatment with conventional DM only began to form fibers beginning at 72-hours post treatment. A fusion index was quantified at 72 hours post treatment (FIG. 9B), in which the percentage of total nuclei present in a myotube (MyHC positive cell with 2 or more nuclei). While conventional DM demonstrated a fusion index of 15%, ERKi however, induced a fusion index of 62%.

Taken together, the results indicate that ERKi is significantly more effective than DM treatment on chicken myoblasts to induce myogenesis. This is supported by the fact that muscle fibers begin to form 48 hours earlier than with DM, and at 72-hours post treatment when ERKi reaches its maximum effect, it demonstrates a 4-fold increase in fusion index compared to DM.

Through the evaluation of gene expression of various transcription factors which are indicative of processes of myoblast differentiation, it was observed that both ERKi and DM treatment induce a differentiation transcriptional program as evident by the reduction in Pax7 RNA expression. While the effect of ERKi on the upregulation of MyoD expression likely occurred prior to the 24-hour timepoint that was collected, its eventual downregulation occurs prior to that of DM, suggesting its earlier regulation. The effect of ERKi treatment is more pronounced on the downregulation of Myf5 expression. Although both DM and ERKi treatment resulted in down regulation of Myf5, the effect of ERKi is stronger across all time points. Similar to MyoD, the maximum effect of ERKi on Myog expression likely occurred prior to 24 hours, as both DM and ERKi increased Myog expression at 24 hours, although in DM its expression continued to rise through 72 hours while ERKi levels of Myog fall by 48 hours post treatment corresponding with the massive formation of fibers (FIG. 9C).

Interestingly, the earlier induction of differentiation, fusion, and myotube formation achieved upon treatment of myoblasts with ERKi as compared with DM is accompanied by earlier maturation of the muscle fibers as evident by the elevated gene expression of various maturation/differentiation markers including the transcription factor mrf4, and myosin heavy chain 1 and 2 (myh1 and myh2) and troponin 3 (tnnt3) (FIG. 10A). At 48 hours post treatment, sarcomeric structure is already apparent by immunofluorescence staining of ERKi induced myotubes as is evident by the classical ladder-like striation of the actinin and troponin signals as well as a phalloidin stain representing actin filaments (FIG. 10B).

### Example 8

### ERK Inhibition Improves Yield of the Produced Fibers

For surface area coverage—equal number of chicken primary myoblasts were seeded in 96 well plates and the following day treated with PM supplemented with 1 uM SCH 772984 (ERKi) or differentiation media (DM). media was replenished daily either with fresh PM or DM (ERKi was not added again). At 72 hours post treatment plates were fixed, and immunostained for myosin heavy chain. Images were captured and analysed for area coverage of the red signal compared to total area per field using ImageJ software.

For cell pellet weights—equal number of chicken primary myoblasts were seeded in 10 cm dishes and the following day treated with PM supplemented with 1 uM SCH 772984 (ERKi) or differentiation media (DM). media was replenished daily either with fresh PM or DM (ERKi was not added again). At 72 hours post treatment media was aspirated and 1 ml of PBS was put in each dish and the cells were scrapped with a rubber policeman scraper. Prior to collection each individual collection tube was weighed on an analytical scale while empty. Then, cell suspensions were collected and spun down in a cooled centrifuge. Supernatant solution was gently aspirated by handheld pipet, and the wet pellet and collection tube was weighed again. The weight of the wet pellet was determined by subtracting the original weight for the relevant tube while empty. This was repeated for 6 replicates per treatment.

For protein yield—equal number of chicken primary myoblasts were seeded in 12-well plates and the following day treated with PM supplemented with 1 uM SCH 772984 (ERKi) or differentiation media (DM). Media was replenished daily either with fresh PM or DM (ERKi was not added again). At 72 hours post treatment media was aspirated and 200 ul of RIPA buffer supplemented with protease inhibitor cocktail was added per well, cells were scraped and lysates were incubated at 4 degrees for 30 minutes. Lysates were then centrifuged to remove unsoluble material and the supernatants were evaluated for total protein by the BCA method.

Results

In order to evaluate parameters of overall yield, meaning the overall amount of total product following the procedure, several methods are used in the industry to date: first, is to evaluate either volume or surface area coverage taken by the muscle fibers produced; second, is to evaluate the mass (weight) of product produced; and third, is to evaluate total protein contained within the final product either by Bradford or BCA assays.

By evaluating the total surface area coverage by myofibers obtained either by ERKi or DM treatment, it was observed that by 48-hours post treatment, ERKi-derived fibers covered 45% of the surface area, while DM fibers only 7%, a 6-fold increase (FIG. 11A). When comparing the overall cell pellet weight achieved 72 hours post treatment, ERKi yielded a 40% increase in cell pellet weight compared to DM (FIG. 11B).

Prior to the experiment, the cell population underwent several rounds of pre-plating to eliminate as many fibroblasts as possible; however, over the 72-hour time-course, the few fibroblasts that remained in starting culture do proliferate and contribute to the overall mass and protein yield at the endpoint. However, despite this fact, it can be estimated that number of starting cells needed to produce 1 kg of product by 72 hours post treatment with ERKi is ˜97 million cells, while DM requires ˜130 million cells (FIG. 11C). And finally, ERKi treated cells had a nearly 4-fold increase in the amount of total protein as compared to cells treated with DM (FIG. 11D).

### Example 9

### ERK Inhibition Improves Fiber Formation in Bovine and Sheep Myoblasts

Bovine myoblasts were seeded in equal number in 96-well plates and the following day treated either with PM, PM supplemented with 0.5 uM SCH 772984 (ERKi) or differentiation media (DM). Media was replenished daily either with fresh PM or DM (ERKi was not added again). At 72 hours post treatment, cells were fixed and stained for myosin heavy chain, and several fields per condition were imaged. Nuclei per fiber was quantified per field to result in a fusion index. For ovine myoblasts—equal number of cells were seeded in an 8 well chamber slide for 24 hours. The following day cells were treated with either with 1 uM SCH 772984 (ERKi) or differentiation media (DM), supplemented with 100 nM of Sir-Actin reagent, then incubated for 4 hours and then the slide was transferred to a heated and CO₂ chambered microscope unit. Several fields of view were selected and live imaging was performed by capturing images of each field every 7 minutes. The series of images for each treatment over a period of 31 hours was stacked into a movie file using ImageJ.

Results

In addition, the present inventors sought to demonstrate the applicability of ERKi on myoblasts from an additional species. Bovine myoblasts were harvested and grown. The effect of ERKi was found to be conserved across species. Following treatment with a single administration of 0.5 uM of ERKi, bovine myoblasts show a 6-fold increase in fusion index as compared to DM at 72 hours post treatment.

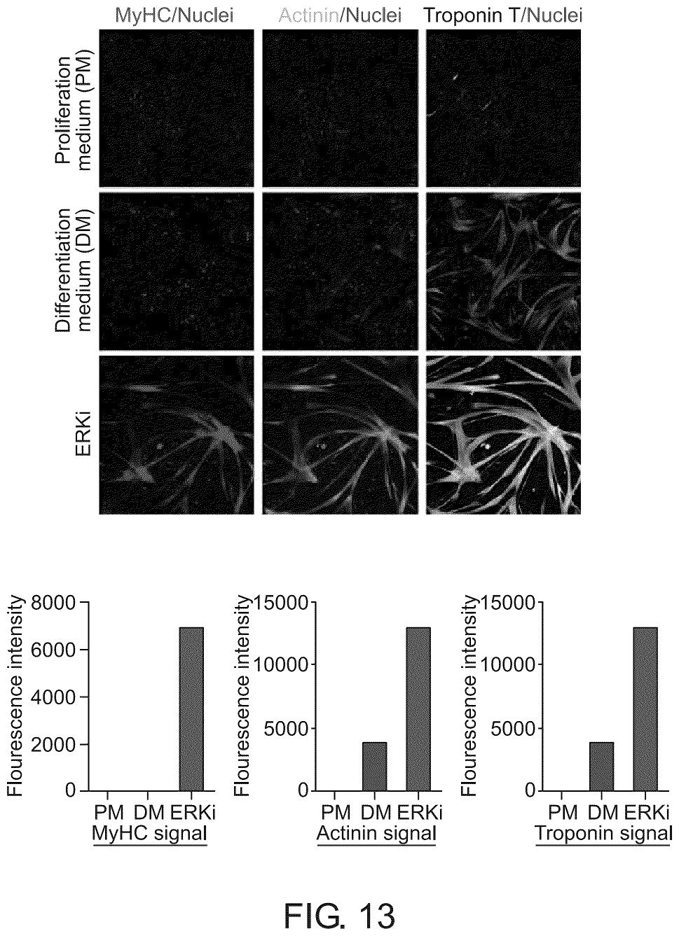

As suggested from both the mouse and chicken data, maturation markers were increased in ERKi induced myotubes compared to DM at 96 hours post-treatment. Despite the fact that myotubes were present in DM by 96 hours, the expression of sarcomeric proteins MyHC, actinin and tropoinin were significantly less than those induced by a ERKi treatment, as evident by the evaluation of the relative signal intensity of the immunofluorescent staining (FIG. 13).

Finally, the present inventors demonstrate that ERKi is similarly effective on sheep derived myoblasts as treatment with 1 μM induce significantly more fusion and myotube formation as compared to treatment with DM (not shown).

### Materials and Methods for EXAMPLES 10-15

Primary chicken myoblast isolation and treatment: chicken myoblasts were isolated from broiler chicken embryos at day 18 from breast and leg muscles by using Trypsin B. Following tissue dissociation, the cell suspension was grown for 3-4 days on 10% Matrigel coated plates. At the first passage, the cells were lifted and pre-plated twice for 30 minutes to enrich for myoblasts and reduce the number of fibroblasts. Then cells were seeded at 8,000/well of optical-96 well plates in proliferation medium. 24 hours after plating, media was aspirated and replaced with the indicated treatment conditions in proliferation media or differentiation media (as indicated). After 24 hours with treatment, the media was aspirated and all wells were replenished with fresh proliferation media or differentiation media without any treatment. This was repeated daily. Cells were fixed at 48 hours after treatment. Compounds were purchased from Cayman Chemicals.

Primary bovine myoblast isolation and treatment: bovine myoblasts were isolated from freshly slaughtered muscle cow muscle with collagenase type II. Following tissue dissociation, the cell suspension was grown for 3-4 days on 10% Matrigel coated plates. At the first passage, the cells were lifted and pre-plated twice for 30 minutes to enrich for myoblasts and reduce the number of fibroblasts. Then cells were seeded at 8,000/well of optical-96 well plates in proliferation medium. 24 hours after plating, media was aspirated and replaced with the indicated treatment conditions in proliferation media. After 24 hours with treatment, the media was aspirated and all wells were replenished with fresh proliferation without any treatment. This was repeated daily. Cells were fixed at 72 hours after treatment. All ERK inhibitors were purchased from Cayman Chemicals.

Microscopy: Fixed samples were imaged using the Nikon Eclipse Ti2 microscope and NIS-Elements imaging software ver.5.11.00. using a 10× objective for the acquisition of MyHC, alpha sarcomeric actinin, and DAPI staining. If necessary, linear adjustment to brightness and contrast were applied using Photoshop.

Immunofluorescent staining: Cells were fixed with ice cold 4% PFA in PBS for 10 minutes, permeabilized with 0.5% Triton X-100 in PBS for 6 minutes, and blocked in PBS with 0.025% tween, 10% normal horse serum and 10% normal goat serum for 1 hour at room temperature. Primary antibody incubation was done in blocking buffer overnight at 4 degrees, with the following antibodies: Myosin Heavy Chain (MyHC, MF20, DSHB hybridoma supernatant 1:10, alpha-actinin (SIGMA A7811). Cells were washed 3 times in PBS with 0.025% tween and then incubated with appropriate secondary antibodies in PBS 1 hour. Nuclei were labeled with DAPI (SIGMA D9542, 5 ug/ml). All fusion indexes and imaging analysis were performed on at least 1000 per technical repeat.

### Example 10

### Various ERK Inhibitors Induce Differentiation and Fusion in Primary Bovine Myoblasts

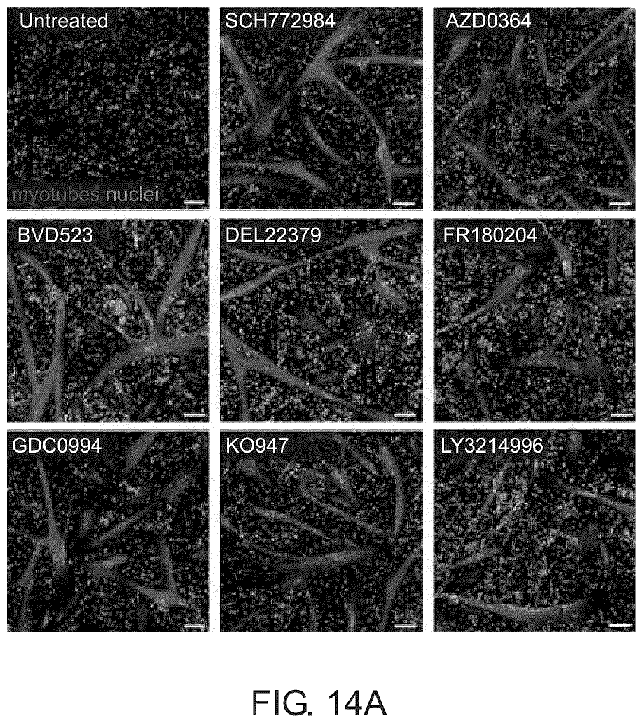

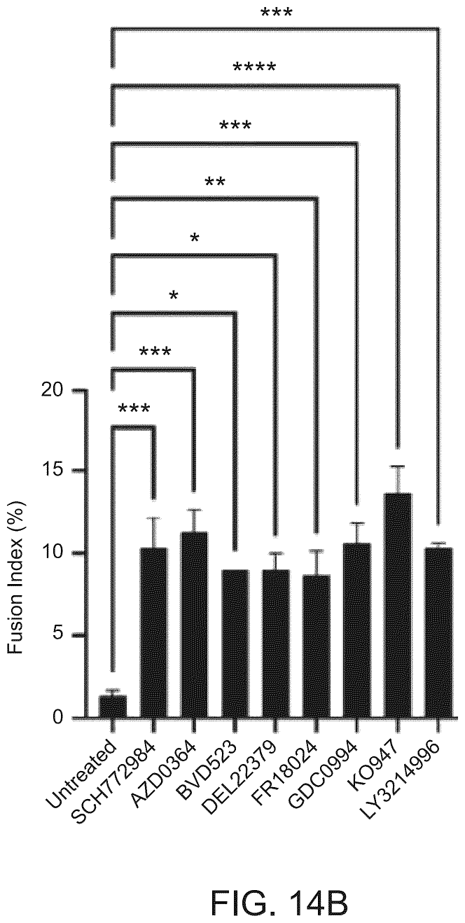

Several ERK inhibitors other than SCH772984 (AZD0364, BVD523, DEL22379, FR180204, GDC0994, K0947, and LY3214996) were compared for their ability to induce differentiation and fusion in primary isolated bovine myoblasts and compared to treatment with 1 uM SCH772984. Quantification of fusion indexes (FIG. 14B) of cells fixed and stained for alpha sarcomeric actinin and DAPI at 72 hours post-treatment (FIG. 14A), indicated that all of the ERK inhibitors tested, when added to proliferation medium at the same concentration as SCH772984 (1 uM), had a similar ability to induce differentiation and fusion in the primary myoblasts.

### Example 11

### Calcium Ionophores Enhance ERK-Inhibitor Induced Differentiation and Fusion in Primary Chicken Myoblasts

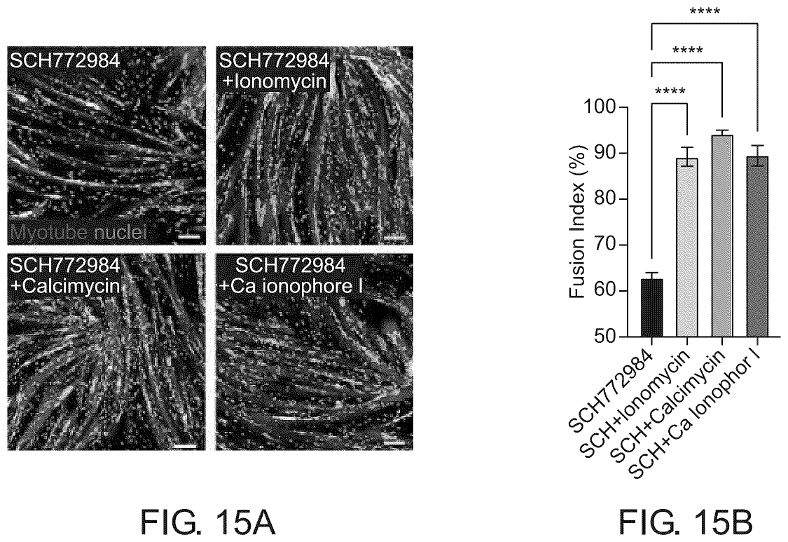

Calcium ionophores can be employed to increase the cytosolic calcium, which is required for CaMKII activation. Thus, the effect of calcium ionophores on ERKi-induced differentiation and fusion phenotype was investigated. Addition of three different calcium ionophores (ionomycin, calcimycin, and calcium ionophore I (AKA CA1001 or ETH1002)) to ERKi administration significantly increased the fusion index of primary chicken myoblasts (in proliferation medium), from 62% in the ERKi treatment alone to 89%, 94%, and 89% respectively for the ionophores (FIGS. 15A and 15B).

### Example 12

### RXR/RAR Agonists Enhance ERK-Inhibitor Induced Differentiation and Fusion in Primary Chick Myoblasts

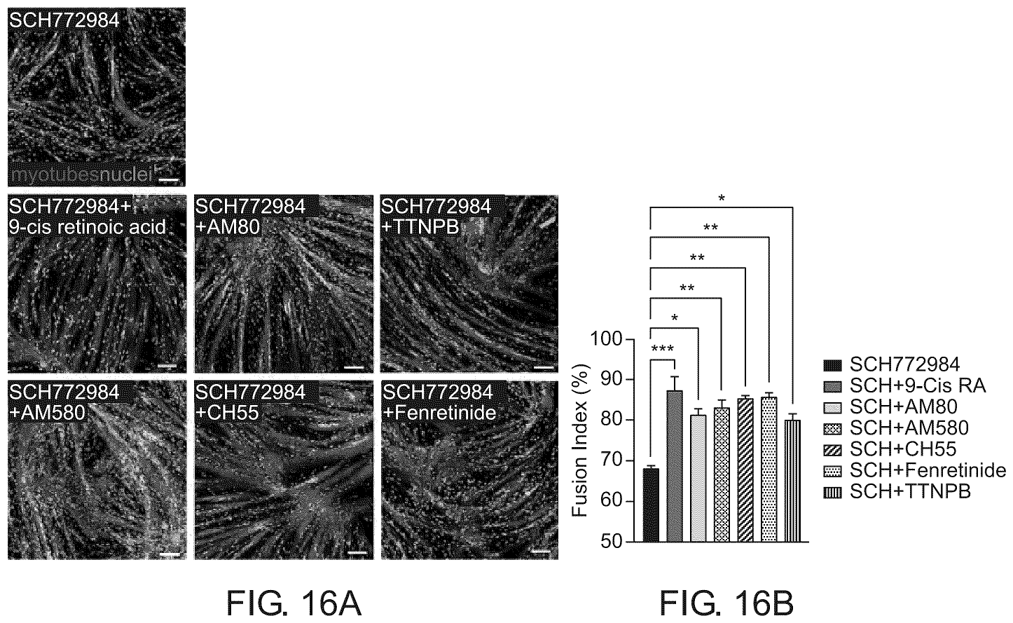

Retinoid X Receptor (RXR) activation is implicated in the CaMKII signaling pathway. The effect of RXR and related Retinoic Acid receptor (RAR) agonists on the ERKi-induced differentiation and fusion phenotype in myoblasts was investigated. Primary chicken myoblasts were treated either with ERK inhibitor alone (SCH772984 1 uM, SCH) or in combination with various RXR/RAR agonists (9-cis retinoic acid, 9-cis RA-200 nM, AM80-200 nM, AM580-100 nM, and CH55-200 nM, TTNPB 200 nM, and Fenretinide 200 nM) in proliferation media. The combination of the RXR/RAR agonists with the ERKi inhibitors significantly increased the fusion index of primary myoblasts. (FIGS. 16A and 16B).

### Example 13

### RYR Agonists Enhance ERK-Inhibitor Induced Differentiation and Fusion in Primary Chick Myoblasts

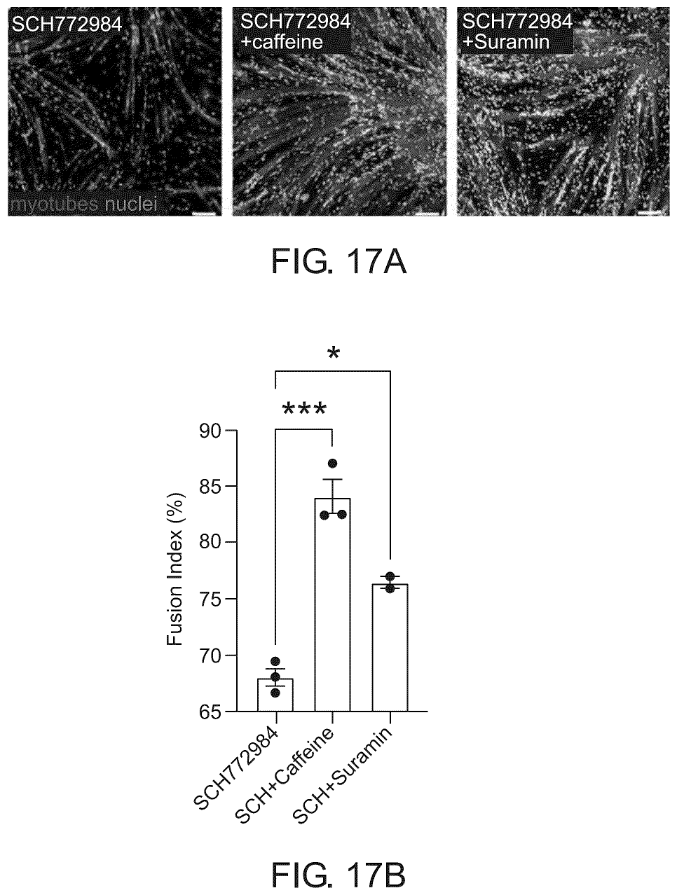

Ryanodine Receptor (RYR) activation is implicated in the CaMKII signaling pathway. The effect of RYR agonists on the ERKi-induced differentiation and fusion phenotype in myoblasts was investigated. Primary chicken myoblasts were treated either with ERK inhibitor alone (SCH772984 1 uM, SCH) or in combination with RYR agonists (Caffeine 2 mM and Suramin 10 μM) in proliferation media. The combination of the RYR agonists with the ERKi inhibitors significantly increased the fusion index of primary myoblasts. (FIGS. 17A and 17B).

### Example 14

### ERK Vs. MEK Inhibition—Effect on Differentiation and Fusion in Primary Chicken Myoblasts

Both Mitogen activated protein kinase (MEK) and ERK are important components of the MAPK pathway. Comparing the effects of 1 and 10 uM of both SCH772984 and the MEK inhibitor (U0126, MEKi) on primary chicken myoblasts either in proliferation or in differentiation media, the superior effect of ERK inhibition (SCH772984) over MEK inhibition (U0126) on chick myoblast differentiation and fusion was clearly observed: In Proliferation media, at 48 hours post treatment the 1 uM dose of SCH772984 induced 59% fusion while the similar dose of the MEKi only induced 16% fusion, and the 10 uM dose of SCH772984 induces 69% fusion while 10 uM of the MEKi induced less fusion (7%) than 1 uM of the MEKi (16%). In Differentiation media, 1 uM of SCH772984 induced 46% fusion while 1 uM of the MEKi induced only 29% fusion. At 10 uM, SCH772984 induced 61% fusion while the MEKi induces only 47% fusion (see FIGS. 18A and 18B).

### Example 15

### Combination of ERK Inhibitor, Ryanodine Receptor Agonist, RXR/RAR Agonist, and Calcium Ionophore: Effect on Induction of Fusion Phenotype in Myoblasts Compared to Treatment with ERK Inhibitor Alone

Interaction of various agents (i.e. RXR/RAR agonists, RYR agonists and Calcium ionophores) capable of modulating the effect of ERK inhibition on myoblast fusion is investigated for. Combination treatments with the various identified molecules from each class is tested for ability to either further enhance myoblast fusion, or shorten the time required to reach comparable level of fusion.

Methods:

Treatment of myoblasts with combinations of molecules: bovine and chicken myoblasts are isolated and cultured as described above. Cells are seeded at 8,000/well of optical-96 well plates in proliferation medium. 24 hours after plating, media is aspirated and replaced with the indicated treatment conditions in proliferation media or differentiation media. Using RXR/RAR, RYR agonists, and Calcium ionophores identified to enhance fusion upon co-treatment with ERK inhibitor, various combinations of 3 to 4 different compounds are tested at different doses. After 24 hours with treatment, the media is aspirated and all wells are replenished with fresh proliferation media without any treatment. This protocol is repeated daily. Cells are fixed for evaluation 72 hours after treatment.

Microscopy: Fixed samples are imaged using the Nikon Eclipse Ti2 microscope and NIS-Elements imaging software ver.5.11.00 using a 10× objective for the acquisition of MyHC, alpha sarcomeric actinin, and DAPI staining. If necessary, linear adjustment to brightness and contrast are applied using Photoshop.

Immunofluorescent staining: Cells are fixed with ice cold 4% PFA in PBS for 10 minutes, permeabilized with 0.5% Triton X-100 in PBS for 6 minutes, and blocked in PBS with 0.025% tween, 10% normal horse serum and 10% normal goat serum for 1 hour at room temperature. Primary antibody incubation is effected in blocking buffer overnight at 4 degrees, with the following antibodies: Myosin Heavy Chain (MyHC, MF20, Dev Stud Hyridoma Bank hybridoma supernatant 1:10), alpha-actinin (SIGMA A7811). Cells are washed 3 times in PBS with 0.025% Tween and then incubated with appropriate secondary antibodies in PBS 1 hour. Nuclei are labeled with DAPI (SIGMA D9542, 5 ug/ml). All fusion indexes and imaging analyses are performed on at least 1000 per technical repeat.

Results: Combinations of agents which provide significant increases in the fusion index at a particular time following exposure, or can significantly reduce the time of incubation for achieving designated level[s] of myoblast fusion are chosen. Synergic combinations are of particular interest.

### Example 16

### Effect of Sequential Treatment of Bovine Myoblasts with ERKi, RXR/RAR Agonists, RYR Agonists and Calcium Ionophores on Fusion Phenotype

The significance of chronology and timing of the addition of agents enhancing ERK-inhibitor-induced myoblast differentiation and fusion is investigated. As the natural timeline of bovine myoblast differentiation and fusion is longer than chicken or mouse myoblasts, sequential administration of RXR/RAR agonists, RYR agonists and calcium ionophores with ERKi is tested. ERK inhibitor is administered at t0, and then either individual RXR/RAR agonists, RYR agonists, and Calcium ionophores, or combinations thereof are administered to the myoblasts at 24, 48, or 72 hours following the initial ERK inhibitor treatment.

Methods:

Primary bovine myoblast isolation and treatment: bovine myoblasts are isolated from freshly slaughtered muscle cow muscle with collagenase type II. Following tissue dissociation, the cell suspension is grown for 3-4 days on 10% Matrigel coated plates. At the first passage, the cells are lifted and pre-plated twice for 30 minutes to enrich for myoblasts and reduce the number of fibroblasts. Then cells are seeded at 8,000/well of optical-96 well plates in proliferation medium. 24 hours after plating, medium is aspirated and replaced with ERKI treatment in proliferation media or differentiation media. Using RXR/RAR, RYR agonists, and Calcium ionophores identified to enhance fusion upon co-treatment with ERK inhibitor, various combinations of 3 to 4 different compounds are tested at different doses, at 24, 48, and 72 hours after initial treatment with ERK inhibitor. All wells are replenished with fresh proliferation media or differentiation media daily with the indicated treatment where indicated. This protocol is repeated daily, with the cells being fixed at 72 hours after treatment.

Microscopy: Fixed samples are imaged using the Nikon Eclipse Ti2 microscope and NIS-Elements imaging software ver.5.11.00, using a 10× objective for the acquisition of MyHC, alpha sarcomeric actinin, and DAPI staining images. Where necessary, linear adjustment to brightness and contrast are applied using Photoshop.

Immunofluorescent staining: Cells are fixed with ice cold 4% PFA in PBS for 10 minutes, permeabilized with 0.5% Triton X-100 in PBS for 6 minutes, and blocked in PBS with 0.025% tween, 10% normal horse serum and 10% normal goat serum for 1 hour at room temperature. Primary antibody incubation is effected in blocking buffer overnight at 4 degrees, with the following antibodies: Myosin Heavy Chain (MyHC, MF20, DSHB hybridoma supernatant 1:10), alpha-actinin (SIGMA A7811). Cells are washed 3 times in PBS with 0.025% Tween and then incubated with appropriate secondary antibodies in PBS for 1 hour. Nuclei are labeled with DAPI (SIGMA D9542, 5 ug/ml). All imaging analyses are performed on at least 1000 per technical repeat.

Results: Sequences of combining agents, which provide significant increases in the fusion index at a particular time following exposure, or can significantly reduce the time of incubation for achieving designated level[s] of myoblast fusion are chosen. Indication of differences of effective chronology of combinations, for the different protocols, are of particular interest.

### Example 17

### Effect of Inhibition of SERCA Channels or Other Calcium Regulators on Myoblast Fusion: Combination with ERK-Inhibitors and/or with RXR/RAR Agonists, RYR Agonist, or Calcium Ionophores

Previous results indicate that ERKi treatment in myoblasts, in addition to activating of RYRs and inducing Calcium release and CaMKII activation, also increases flux through SERCA channels, and activates other calcium regulators. Without wishing to be limited to one particular hypothesis, it is considered possible that their upregulation is a compensation mechanism within the cells, in an effort to balance the amount of available Calcium and return Calcium to the ER. Further inhibition of the affected channels may facilitate a prolonged accumulation of intracellular calcium, a stronger activation of CaMKII and enhance fusion to even a greater degree than treatment with ERKi alone, or ERKi in combination with RXR/RAR agonists, and/or RYR agonists, and/or Calcium ionophores.

Methods:

Primary chicken myoblast isolation and treatment. Chicken myoblasts are isolated from broiler chicken embryos at day 18 from breast and leg muscles by using Trypsin B. Following tissue dissociation, the cell suspension is grown for 3-4 days on Matrigel coated plates. At the first passage, the cells are lifted and pre-plated twice for 30 minutes to enrich for myoblasts and reduce the number of fibroblasts. Then cells are seeded at 8,000/well of optical-96 well plates in proliferation medium. 24 hours after plating, medium is aspirated and replaced with the either proliferation media or differentiation media with SCH772984 alone, or SCH772984 in combination with SERCA inhibitors/or other calcium reuptake modulators, or the latter in combination with RXR/RAR agonists, and/or RYR agonists, and/or Calcium ionophores. After 24 hours with treatment, the medium is aspirated and all wells are replenished with fresh proliferation media or differentiation media without any treatment. This is repeated daily. Cells are fixed at 24, 48, 72 and 96 hours after treatment.

Primary bovine myoblast isolation and treatment: Bovine myoblasts are isolated from freshly slaughtered muscle cow muscle with collagenase type II. Following tissue dissociation, the cell suspension is grown for 3-4 days on Matrigel coated plates. At the first passage, the cells are lifted and pre-plated twice for 30 minutes to enrich for myoblasts and reduce the number of fibroblasts. Then cells are seeded at 8,000/well of optical-96 well plates in proliferation medium. 24 hours after plating, medium is aspirated and replaced with the indicated treatment conditions in proliferation media or differentiation media. Using RXR/RAR, RYR agonists, and Calcium ionophores previously identified to enhance fusion upon co-treatment with ERK inhibitor, various combinations of 3 to 4 different compounds are tested at different doses, at 24, 48, and 72 hours after initial treatment with ERKi as indicated. All wells are replenished with fresh proliferation media or differentiation media daily with the indicated treatment where indicated. The protocol is repeated daily, and the cells are fixed at 24, 48, 72 and 96 hours after completion of the treatment.

Microscopy: Fixed samples are imaged using the Nikon Eclipse Ti2 microscope and NIS-Elements imaging software ver.5.11.00, using a 10× objective for the acquisition of MyHC, alpha sarcomeric actinin, and DAPI staining. If necessary, linear adjustments to brightness and contrast are applied using Photoshop.

Immunofluorescent staining: Cells are fixed with ice cold 4% PFA in PBS for 10 minutes, permeabilized with 0.5% Triton X-100 in PBS for 6 minutes, and blocked in PBS with 0.025% Tween, 10% normal horse serum and 10% normal goat serum for 1 hour at room temperature. Primary antibody incubation is done in blocking buffer overnight at 4 degrees, with the following antibodies: Myosin Heavy Chain (MyHC, MF20, DSHB hybridoma supernatant 1:10), alpha-actinin (SIGMA A7811). Cells are washed 3 times in PBS with 0.025% Tween and then incubated with appropriate secondary antibodies in PBS 1 hour. Nuclei are labeled with DAPI (SIGMA D9542, 5 ug/ml). All imaging analyses is performed on at least 1000 per technical repeat.

### Example 18

### Effect of ERK Inhibition, Alone or in Combination with RXR/RAR or RYR Agonists, Calcium Ionophores, and Inhibitors of SERCA/Calcium Reuptake Channels, on Teleost (Fish)-Derived Myoblast Differentiation and Fusion

The ability of ERK inhibitors, alone and in combination with other molecules affecting the ERK-CaMKII signaling pathway to induce fusion in teleost (fish) myoblasts is investigated, and compared to/contrasted with their effect on mouse, chicken, and bovine myoblast development.

Methods:

Primary trout and zebrafish myoblast isolation and treatment: trout and zebrafish myoblasts are isolated from freshly sacrificed mature fish either using Trypsin B or collagenase digestion. Following tissue dissociation, the cell suspension is grown for 3-4 days on Matrigel coated plates. At the first passage, the cells are lifted and pre-plated twice for 30 minutes to enrich for myoblasts and reduce the number of fibroblasts. Then the cells are seeded at 8,000/well of optical-96 well plates in proliferation medium. 24 hours after plating, medium is aspirated and replaced with the either proliferation media or differentiation media with SCH77s984 alone, or SCH772984 in co-treatment with various combinations/doses of SERCA inhibitors/or calcium reuptake modulators, RXR/RAR agonists, and/or RYR agonists, and/or Calcium ionophores (as indicated). After 24 hours with treatment, the media is aspirated and all wells replenished with fresh proliferation media or differentiation media without any treatment. This protocol is repeated daily, and the cells are fixed 24, 48, 72 and 96 hours after treatment.

Microscopy: Fixed samples are imaged using the Nikon Eclipse Ti2 microscope and NIS-Elements imaging software ver.5.11.00 using a 10× objective for the acquisition of MyHC, alpha sarcomeric actinin, and DAPI staining. If necessary, linear adjustment to brightness and contrast is applied using Photoshop.

Immunofluorescent staining: Cells are fixed with ice cold 4% PFA in PBS for 10 minutes, permeabilized with 0.5% Triton X-100 in PBS for 6 minutes, and blocked in PBS with 0.025% tween, 10% normal horse serum and 10% normal goat serum for 1 hour at room temperature. Primary antibody incubation is done in blocking buffer overnight at 4 degrees, with the following antibodies: Myosin Heavy Chain (MyHC, MF20, DSHB hybridoma supernatant 1:10), alpha-actinin (SIGMA A7811). Cells are washed 3 times in PBS with 0.025% Tween and then incubated with appropriate secondary antibodies in PBS 1 hour. Nuclei are labeled with DAPI (SIGMA D9542, 5 ug/ml). All imaging analyses are performed on at least 1000 per technical repeat.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of inducing multinucleated myotube formation, the method comprising contacting myogenic precursor cells from a farmed animal with at least one molecule selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist, a calcium ionophore and a Calmodulin-dependent Protein Kinase II (CaMKII) activator.

2. The method of claim 1, wherein said at least one molecule is an Extracellular Regulated Signaling Kinase (ERK1/2) inhibitor and/or an upregulator of intracellular Ca 2+.

3. The method of claim 1, wherein said ERK1/2 inhibitor is an ERK1/2 specific inhibitor.

4. The method of claim 1, wherein:
(a) said ERK1/2 inhibitor is selected from the group consisting of MK-8353 (SCH900353), SCH772984, CC-90003, Corynoxeine, ERK1/2 inhibitor 1, magnolin, ERK IN-1, ERK IN-2, ERK IN-3, LY3214996, Ravoxertinib, Ravoxertinib hydrochloride, VX-11e, FR 180204, Ulixertinib, Ulixertinib hydrochloride, ADZ0364, K0947, FRI-20 (ON-01060), Bromacetoxycalcidiol (B3CD), BVD523, DEL22379, FR180204, GDC0994, K0947, AEZ-131(AEZS-131), AEZS-136, AZ-13767370, BL-EI-001, LTT, ASTX-029, TCS ERK 11e and CAY10561,
(b) said MEK1 inhibitor is selected from the group consisting of Trametinib, PD98059, U0126 (U0126-EtOH), PD0325901, Selumetinib (AZD6244), Cobimetinib (GDC-0973, RG7420), Binimetinib (MEK162), CI-1040 (PD 184352), Refametinib (BAY 869766; RDEA119), Pimasertib (AS703026), Selumetinib (AZD6244), Cobimetinib hemifumarate, GDC-0623 (RG 7421), R04987655, AZD8330, (ARRY-424704), SL327, MEK inhibitor, PD318088, Cobimetinib racemate (GDC-0973 racemate; XL518 racemate) and EBI-1051,
(c) said RXR/RAR agonist is selected from the group consisting of CD3254, Docosahexaenoic acid, LG100268, SR11237, AC261066, AC55649, Adapalene, BMS961, CD1530, CD2314, CD437, BMS453, EC23, all-trans retinoic acid, all-trans-4-hydroxy retinoic acid, all-trans retinoic acid-d5, cyantraniliprole, Vitamin A, all-trans retinol, LG100754, Beta Carotene, beta-apo-13 carotene, lycopene, all-trans-5,6-epoxy retinoic acid, all-transe-13,14-Dihydroretinol, Retinyl Acetate, Hanokiol, Valerenic acid, HX630, HX600, LG101506, 9cUAB30, AGN194204, LG101305, UVI3003, Net-41B, CBt-PMN, XCT0135908, PA024, methoprene acid, 9-cis retinoic acid, AM80, AM580, and CH55, TTNPB, and Fenretinide, LG-100064, Fluorobexarotene (compound 20), Bexarotene (LGD1069), Bexarotene D4, NBD-125 (B-12), LGD1069 D4 and 9-cis-Retinoic acid (ALRT1057),
(d) said RYR1, RYR3 agonist is selected from the group consisting of Caffeine, Chlorocresol, CHEBI:67113, chlorantraniliprole, S107hydrochloride, JTV519, Trifluoperazine (T FP), Xanthines, Suramin, Suramin sodium, NAADP tetrasodium salt, S100A1, Cyclic ADP-Ribose (ammonium salt), pentifylline, 4-chloro-3-methylphenol (4-chloro-m-cresol), tetraniliprole, trifluoperazine (TFP), cyclaniliprole and Cyantraniliprole,
(e) said upregulator of intracellular Ca2+ is selected from the group consisting of NAADP tetrasodium salt, Cyclic ADP-Ribose, 4-bromo A23187, Ionomycin, A23187 and isoproterenol, and
(f) said CaMKII agonist is selected from the group consisting of Calcium, Calmodulin, CALP1 and CALP3.

5. The method of claim 3, wherein said ERK1/2 inhibitor is SCH772984.

6. The method of claim 1, wherein said myogenic precursor cells are selected from the group consisting of myoblasts, satellite cells, muscle side population (mSP) cells, muscle-derived stem cells (MDSCs), mesenchymal stem cells (MSCs), muscle-derived pericytes, embryonic stem cells (ESCs), induced muscle progenitor cells (iMPCs) and Induced Pluripotent Stem cells (iPSCs).

7. The method of claim 1, wherein said myogenic precursor cells express MyoD, Pax3 and Pax7, or the corresponding orthologs thereof.

8. The method of claim 1, wherein said myogenic precursor cells are myoblasts.

9. The method of claim 1, wherein said myogenic precursor cells are from a biopsy of said farmed animal, and, optionally, a muscle biopsy.

10. The method of claim 9, wherein said myogenic precursor cells are isolated from said biopsy by enzymatic dissociation and/or mechanical dissociation.

11. The method of claim 1, wherein said myogenic progenitor cells are undifferentiated myogenic precursor cells cultured in proliferation medium prior to inducing said multinucleated myotube formation.

12. The method of claim 11, wherein said proliferation medium is devoid of molecules selected from the group consisting of an Extracellular Regulated Signaling Kinase (ERK1/2) inhibitor, a Mitogen-Activated Protein Kinase Kinase 1 (MEK1) inhibitor, a Fibroblast Growth Factor (FGF) inhibitor, a Transforming Growth Factor-Beta (TGF-Beta) inhibitor, a Retinoid-X Receptor (RXR) agonist, a Retinoid-X Receptor (RXR) activator, a Retinoic Acid Receptor (RAR) agonist, a Retinoic Acid Receptor (RAR) activator, a Ryanodine Receptor (RYR1, RYR3) agonist, a Ryanodine Receptor (RYR1, RYR3) activator, an upregulator of intracellular Ca 2+, a Calmodulin-dependent Protein Kinase II (CaMKII) agonist, calcium ionophore and a Calmodulin-dependent Protein Kinase II (CaMKII) activator.

13. The method of claim 1, wherein said myogenic progenitor cells are myogenic precursor cells cultured in a differentiation medium prior to inducing said multinucleated myotube formation.

14. The method of claim 1, effected in a single vessel and optionally effected by supplementing said medium with any of said molecules.

15. The method of claim 1, effected in the presence of serum or serum replacement at an amount which allows cell proliferation and/or under normoxic conditions.

16. The method of claim 1, wherein:
(a) said multinucleated myotubes comprise at least three nuclei, and/or
(b) said multinucleated myotubes express myogenic differentiation and fusion factors selected from the group consisting of MyoD, MyoG, Mymk and Mymx, and/or
(c) said inducing multinucleated myotubes results in increased fraction of MYOG-positive nuclei, as compared to nuclei of myogenic progenitor cells cultured in differentiation medium without said at least one molecule, and/or
(d) said inducing multinucleated myotube formation results in classical ladder-like striation of actinin and troponin signals and/or phalloidin staining representing actin filaments, and/or
(e) said inducing multinucleated myotube formation results in classical ladder-like striation of actinin and troponin signals and/or phalloidin staining representing actin filaments, and/or
(f) said multinucleated myotube formation comprises mononucleated myoblast-myotube fusion and/or expansion of bi- and tri-nucleated myotubes into large multinucleated fibers.

17. A cultured meat composition comprising multinucleated myotubes produced by the method of claim 1.

18. A cell culture medium for preparing multinucleated myotubes from myogenic precursor cells, the culture medium comprising a base medium and an Extracellular Regulated Signaling Kinase (ERK1/2) inhibitor.
